SEQUENCE LISTING (124 sequences)

SEQ ID NO: 1 - DNA, 30 bp, Artificial
  Synthetic
acgttggatgaaggctctgaagacctgttc

SEQ ID NO: 2 - DNA, 30 bp, Artificial
  Synthetic
acgttggatgatccggattatcgggaagag

SEQ ID NO: 3 - DNA, 30 bp, Artificial
  Synthetic
acgttggatgcttggtgatgatatcgtggg

SEQ ID NO: 4 - DNA, 30 bp, Artificial
  Synthetic
acgttggatgtcttcttcctgcctgatgag

SEQ ID NO: 5 - DNA, 29 bp, Artificial
  Synthetic
acgttggatgaaacggtcgcttcgacgtg

SEQ ID NO: 6 - DNA, 28 bp, Artificial
  Synthetic
acgttggatggggcagaaggaagagttc

SEQ ID NO: 7 - DNA, 30 bp, Artificial
  Synthetic
acgttggatgtacaggtgtctgcctcctga

SEQ ID NO: 8 - DNA, 30 bp, Artificial
  Synthetic
acgttggatgaagagggtctgtcaacatgg

SEQ ID NO: 9 - DNA, 30 bp, Artificial
  Synthetic
acgttggatgatagccatgaccatgctgag

SEQ ID NO: 10 - DNA, 30 bp, Artificial
  Synthetic
acgttggatgggccatttgtttccctcttc

SEQ ID NO: 11 - DNA, 30 bp, Artificial
  Synthetic
acgttggatgttgagatagatcaagggatg

SEQ ID NO: 12 - DNA, 30 bp, Artificial
  Synthetic
acgttggatggtccgggttctgtgaatatg

SEQ ID NO: 13 - DNA, 30 bp, Artificial
  Synthetic
acgttggatgagcatacttagactactacc

SEQ ID NO: 14 - DNA, 30 bp, Artificial
  Synthetic
acgttggatgcacactcaccagggaaaatg

SEQ ID NO: 15 - DNA, 30 bp, Artificial
  Synthetic
acgttggatgattccatggaggctggatag

SEQ ID NO: 16 - DNA, 30 bp, Artificial
  Synthetic
acgttggatggacaacactactaaggcttc

SEQ ID NO: 17 - DNA, 30 bp, Artificial
  Synthetic
acgttggatgactcacagagcacattcacg

SEQ ID NO: 18 - DNA, 30 bp, Artificial
  Synthetic
acgttggatgtgtcactcgagatcttgagg

SEQ ID NO: 19 - DNA, 30 bp, Artificial
  Synthetic
acgttggatgaggcggcgtccgcggagaca

SEQ ID NO: 20 - DNA, 29 bp, Artificial
  Synthetic
acgttggatgctcggccgctcttctgtcc

SEQ ID NO: 21 - DNA, 30 bp, Artificial
  Synthetic
acgttggatgttacctaaacagggagagcg

SEQ ID NO: 22 - DNA, 29 bp, Artificial
  Synthetic
acgttggatgaagcctgcaaccggaagtg

SEQ ID NO: 23 - DNA, 29 bp, Artificial
  Synthetic
acgttggatgctcaggcggccttgcactc

SEQ ID NO: 24 - DNA, 29 bp, Artificial
  Synthetic
acgttggatgaggcgcgggagcactcaga

SEQ ID NO: 25 - DNA, 30 bp, Artificial
  Synthetic
acgttggatggctccacagcatcaagattc

SEQ ID NO: 26 - DNA, 30 bp, Artificial
  Synthetic
acgttggatgttccatttcctcaccctcag

SEQ ID NO: 27 - DNA, 30 bp, Artificial
  Synthetic
acgttggatgggttcaagaagtcatacccc

SEQ ID NO: 28 - DNA, 30 bp, Artificial
  Synthetic
acgttggatgagctctgtcccttggatgtc

SEQ ID NO: 29 - DNA, 30 bp, Artificial
  Synthetic
acgttggatgcgacctgtccttctcaaaac

SEQ ID NO: 30 - DNA, 30 bp, Artificial
  Synthetic
acgttggatggaataacaggcagactctcc

SEQ ID NO: 31 - DNA, 30 bp, Artificial
  Synthetic
acgttggatggaaatgtcctccagcatggg

SEQ ID NO: 32 - DNA, 30 bp, Artificial
  Synthetic
acgttggatgaccctgctccaccgcatgta

SEQ ID NO: 33 - DNA, 30 bp, Artificial
  Synthetic
acgttggatgtgatcttgttcaccttgccg

SEQ ID NO: 34 - DNA, 30 bp, Artificial
  Synthetic
acgttggatgcgaggtgacgtttgacattg

SEQ ID NO: 35 - DNA, 30 bp, Artificial
  Synthetic
acgttggatgcttcagtatatcttggattg

SEQ ID NO: 36 - DNA, 30 bp, Artificial
  Synthetic
acgttggatggttatgccacttagatgagg

SEQ ID NO: 37 - DNA, 30 bp, Artificial
  Synthetic
acgttggatgggagtcaatttatgcagcag

SEQ ID NO: 38 - DNA, 30 bp, Artificial
  Synthetic
acgttggatgcatcgttattggcaggaagc

SEQ ID NO: 39 - DNA, 30 bp, Artificial
  Synthetic
acgttggatgagcccaagaagtttgaactc

SEQ ID NO: 40 - DNA, 30 bp, Artificial
  Synthetic
acgttggatgaggttgctgttctcaaagtg

SEQ ID NO: 41 - DNA, 30 bp, Artificial
  Synthetic
acgttggatggaggtcaggtggatgtttac

SEQ ID NO: 42 - DNA, 30 bp, Artificial
  Synthetic
acgttggatgaccccaagatgcatcttgcc

SEQ ID NO: 43 - DNA, 30 bp, Artificial
  Synthetic
acgttggatgggcaactagcctaaaaaccc

SEQ ID NO: 44 - DNA, 30 bp, Artificial
  Synthetic
acgttggatgcagagtgcggaataaaaggc

SEQ ID NO: 45 - DNA, 29 bp, Artificial
  Synthetic
acgttggatgtgaggtagacaccgcctcc

SEQ ID NO: 46 - DNA, 30 bp, Artificial
  Synthetic
acgttggatgaagagacgtgtaggaagccc

SEQ ID NO: 47 - DNA, 29 bp, Artificial
  Synthetic
acgttggatgcagacattcacaattgatt

SEQ ID NO: 48 - DNA, 30 bp, Artificial
  Synthetic
acgttggatggatagttccaaacatgtgcg

SEQ ID NO: 49 - DNA, 30 bp, Artificial
  Synthetic
acgttggatgtaacgcccctcacagttcac

SEQ ID NO: 50 - DNA, 30 bp, Artificial
  Synthetic
acgttggatgactccaggctggaggaaatg

SEQ ID NO: 51 - DNA, 30 bp, Artificial
  Synthetic
acgttggatgcacagagagagtctggacac

SEQ ID NO: 52 - DNA, 30 bp, Artificial
  Synthetic
acgttggatgtcttggtctttccctcatcc

SEQ ID NO: 53 - DNA, 17 bp, Artificial
  Synthetic
tcacgatgccgacgaag

SEQ ID NO: 54 - DNA, 18 bp, Artificial
  Synthetic
tcacgatgccgacgaaga

SEQ ID NO: 55 - DNA, 19 bp, Artificial
  Synthetic
ggcgtgggtgagttcattt

SEQ ID NO: 56 - DNA, 20 bp, Artificial
  Synthetic
ggcgtgggtgagttcattta

SEQ ID NO: 57 - DNA, 20 bp, Artificial
  Synthetic
gtgctgcaggccccagatga

SEQ ID NO: 58 - DNA, 21 bp, Artificial
  Synthetic
gtgctgcaggccccagatgag

SEQ ID NO: 59 - DNA, 22 bp, Artificial
  Synthetic
cggcctcctgacccttccatcc

SEQ ID NO: 60 - DNA, 23 bp, Artificial
  Synthetic
cggcctcctgacccttccatccc

SEQ ID NO: 61 - DNA, 22 bp, Artificial
  Synthetic
ccacacatatggtggttcataa

SEQ ID NO: 62 - DNA, 23 bp, Artificial
  Synthetic
ccacacatatggtggttcataac

SEQ ID NO: 63 - DNA, 23 bp, Artificial
  Synthetic
tagatcaagggatgatatcaact

SEQ ID NO: 64 - DNA, 24 bp, Artificial
  Synthetic
tagatcaagggatgatatcaacta

SEQ ID NO: 65 - DNA, 24 bp, Artificial
  Synthetic
catacttagactactacctcgatg

SEQ ID NO: 66 - DNA, 25 bp, Artificial
  Synthetic
catacttagactactacctcgatga

SEQ ID NO: 67 - DNA, 28 bp, Artificial
  Synthetic
ctttcctcttacctatccctacttcccc

SEQ ID NO: 68 - DNA, 29 bp, Artificial
  Synthetic
ctttcctcttacctatccctacttccccc

SEQ ID NO: 69 - DNA, 16 bp, Artificial
  Synthetic
acattcacggtcacct

SEQ ID NO: 70 - DNA, 17 bp, Artificial
  Synthetic
acattcacggtcacctc

SEQ ID NO: 71 - DNA, 16 bp, Artificial
  Synthetic
cgcggagacacccatc

SEQ ID NO: 72 - DNA, 17 bp, Artificial
  Synthetic
cgcggagacacccatcc

SEQ ID NO: 73 - DNA, 17 bp, Artificial
  Synthetic
cgacgaaggagggaaat

SEQ ID NO: 74 - DNA, 18 bp, Artificial
  Synthetic
cgacgaaggagggaaatc

SEQ ID NO: 75 - DNA, 19 bp, Artificial
  Synthetic
tcctcgctctcgcgccgcc

SEQ ID NO: 76 - DNA, 20 bp, Artificial
  Synthetic
tcctcgctctcgcgccgccc

SEQ ID NO: 77 - DNA, 19 bp, Artificial
  Synthetic
ccaagacttaagttttgct

SEQ ID NO: 78 - DNA, 20 bp, Artificial
  Synthetic
ccaagacttaagttttgctc

SEQ ID NO: 79 - DNA, 19 bp, Artificial
  Synthetic
ggaccccaacccaagagaa

SEQ ID NO: 80 - DNA, 20 bp, Artificial
  Synthetic
ggaccccaacccaagagaaa

SEQ ID NO: 81 - DNA, 20 bp, Artificial
  Synthetic
gaaacttgggagaacattgt

SEQ ID NO: 82 - DNA, 21 bp, Artificial
  Synthetic
gaaacttgggagaacattgtc

SEQ ID NO: 83 - DNA, 23 bp, Artificial
  Synthetic
ttcttcccccaccagtggctatc

SEQ ID NO: 84 - DNA, 24 bp, Artificial
  Synthetic
ttcttcccccaccagtggctatca

SEQ ID NO: 85 - DNA, 23 bp, Artificial
  Synthetic
acaacttgccggtgctcttgtcc

SEQ ID NO: 86 - DNA, 24 bp, Artificial
  Synthetic
acaacttgccggtgctcttgtcca

SEQ ID NO: 87 - DNA, 24 bp, Artificial
  Synthetic
gattgatttgagataagtcatatc

SEQ ID NO: 88 - DNA, 25 bp, Artificial
  Synthetic
gattgatttgagataagtcatatcc

SEQ ID NO: 89 - DNA, 25 bp, Artificial
  Synthetic
cagaaaaaaaaatcctttgaaagac

SEQ ID NO: 90 - DNA, 26 bp, Artificial
  Synthetic
cagaaaaaaaaatcctttgaaagaca

SEQ ID NO: 91 - DNA, 26 bp, Artificial
  Synthetic
ggtgcagatctaaatactttaggctg

SEQ ID NO: 92 - DNA, 27 bp, Artificial
  Synthetic
ggtgcagatctaaatactttaggctga

SEQ ID NO: 93 - DNA, 17 bp, Artificial
  Synthetic
gagcagcaggtttgagg

SEQ ID NO: 94 - DNA, 18 bp, Artificial
  Synthetic
gagcagcaggtttgaggg

SEQ ID NO: 95 - DNA, 19 bp, Artificial
  Synthetic
taaaaacccggttctcaac

SEQ ID NO: 96 - DNA, 20 bp, Artificial
  Synthetic
taaaaacccggttctcaaca

SEQ ID NO: 97 - DNA, 21 bp, Artificial
  Synthetic
ctccgcctggtgaggtctccc

SEQ ID NO: 98 - DNA, 22 bp, Artificial
  Synthetic
ctccgcctggtgaggtctccca

SEQ ID NO: 99 - DNA, 23 bp, Artificial
  Synthetic
ttcttacaacacaaaatcaaatc

SEQ ID NO: 100 - DNA, 24 bp, Artificial
  Synthetic
ttcttacaacacaaaatcaaatca

SEQ ID NO: 101 - DNA, 16 bp, Artificial
  Synthetic
acttccgtcctccacc

SEQ ID NO: 102 - DNA, 17 bp, Artificial
  Synthetic
acttccgtcctccacca

SEQ ID NO: 103 - DNA, 17 bp, Artificial
  Synthetic
agtctggacacgtgggg

SEQ ID NO: 104 - DNA, 18 bp, Artificial
  Synthetic
agtctggacacgtgggga

SEQ ID NO: 105 - DNA, 29 bp, Artificial
  Synthetic
acgttggatggtctgttgactcttttggc

SEQ ID NO: 106 - DNA, 30 bp, Artificial
  Synthetic
acgttggatgtggtgatcacccaaggcttc

SEQ ID NO: 107 - DNA, 30 bp, Artificial
  Synthetic
acgttggatgcatagagcttaagcgtctcc

SEQ ID NO: 108 - DNA, 30 bp, Artificial
  Synthetic
acgttggatgtgatccttctggtggtcatc

SEQ ID NO: 109 - DNA, 30 bp, Artificial
  Synthetic
acgttggatgtcagtccctcctgggctcta

SEQ ID NO: 110 - DNA, 30 bp, Artificial
  Synthetic
acgttggatgagaagagtcagacggaatcg

SEQ ID NO: 111 - DNA, 30 bp, Artificial
  Synthetic
acgttggatggactcttgcacatcactacc

SEQ ID NO: 112 - DNA, 30 bp, Artificial
  Synthetic
acgttggatgagtgtaggtcttggtgaagc

SEQ ID NO: 113 - DNA, 19 bp, Artificial
  Synthetic
tggccttttcagattctgg

SEQ ID NO: 114 - DNA, 20 bp, Artificial
  Synthetic
tggccttttcagattctggc

SEQ ID NO: 115 - DNA, 23 bp, Artificial
  Synthetic
aagcgtctccaggaaaatcataa

SEQ ID NO: 116 - DNA, 24 bp, Artificial
  Synthetic
aagcgtctccaggaaaatcataac

SEQ ID NO: 117 - DNA, 24 bp, Artificial
  Synthetic
tgggatctaggcagagccactggg

SEQ ID NO: 118 - DNA, 25 bp, Artificial
  Synthetic
tgggatctaggcagagccactgggc

SEQ ID NO: 119 - DNA, 20 bp, Artificial
  Synthetic
cccatcactacctgcagttt

SEQ ID NO: 120 - DNA, 21 bp, Artificial
  Synthetic
cccatcactacctgcagtttc

SEQ ID NO: 121 - DNA, 20 bp, Artificial
  Synthetic
tggccttttcagattctggg

SEQ ID NO: 122 - DNA, 24 bp, Artificial
  Synthetic
aagcgtctccaggaaaatcataat

SEQ ID NO: 123 - DNA, 25 bp, Artificial
  Synthetic
tgggatctaggcagagccactgggt

SEQ ID NO: 124 - DNA, 21 bp, Artificial
  Synthetic
cccatcactacctgcagtttt

(19) United States
(12) Patent Application Publication
(10) Pub. No.: US 2010/0267025 A1
(43) Pub. Date: Oct. 21, 2010

(54) METHODS AND COMPOSITIONS FOR THE ASSESSMENT OF CARDIOVASCULAR FUNCTION AND DISORDERS

(76) Inventor: Robert Peter Young, Auckland (NZ)

Correspondence Address: DAVIS WRIGHT TREMAINE LLP - San Francisco, 505 MONTGOMERY STREET, SUITE 800, SAN FRANCISCO, CA, 94111, US

(21) Appl. No.: 12/518,864

(22) PCT Filed: Nov. 10, 2006
(86) PCT No.: PCT/NZ06/00292

§ 371 Date: Jan. 8, 2010

(30) Foreign Application Priority Data
Nov. 10, 2005 (NZ) 543520
Dec. 6, 2005 (NZ) 543985
Sep. 15, 2006 (NZ) 549951

(51) Int. Cl. C12Q 1/68

(52) U.S. Cl. 435/6

(57) ABSTRACT

The present invention provides methods for the assessment of risk of developing acute coronary syndrome (ACS) in smokers and non-smokers using analysis of genetic polymorphisms. The present invention also relates to the use of genetic polymorphisms in assessing a subject's risk of developing ACS. Nucleotide probes and primers, kits, and microarrays suitable for such assessment are also provided.

### FIELD OF THE INVENTION

The present invention is concerned with methods for assessment of vascular function and/or disorders, and in particular for diagnosing predisposition to and/or severity of coronary artery disease and particularly acute coronary syndrome (ACS) using analysis of genetic polymorphisms and altered gene expression. The present invention is also concerned with methods for diagnosing predisposition to and/or severity of ACS-associated impaired vascular function.

### BACKGROUND OF THE INVENTION

Coronary artery disease (CAD), also known as coronary heart disease or arteriosclerotic heart disease, is the leading cause of death in the United States. According to the American Heart Association, about every 29 seconds someone in the US suffers from a CAD-related event, and about every minute someone dies from such an event. The lifetime risk of having coronary heart disease after age 40 is 49% for men and 32% for women. As women age, the risk increases almost to that of men. Furthermore, the total annual cost of CAD in the United States is approximately US$130 billion.

The cardiovascular disorders that underlie CAD can be divided into two groups, as indeed can the sufferers of such disorders. This is thought to reflect different etiology of the disorders. The disorders of the first group, herein referred to as “Stable CAD”, are degenerate in nature and include the late onset and exertional anginas. Stable CAD typically afflicts older persons, and is associated with age (65 and greater), high blood pressure, diabetes, high cholesterol levels (specifically, high LDL cholesterol and low HDL cholesterol), lack of physical activity or exercise, and obesity.

The disorders of the second group, herein referred to as acute coronary syndrome (ACS), are believed to be associated with inflammation, plaque instability, and/or smoking. ACS includes myocardial infarction and unstable angina. The Applicants believe, without wishing to be bound by any theory, that, more so than in Stable CAD, genetic risk factors are significant in susceptibility to and/or severity of ACS.

Moreover, the Applicants believe, again without wishing to be bound by any theory, that the biomarkers associated with Stable CAD are unlikely to be associated with, or predictive of, risk of ACS, and vice versa.

It would be desirable and advantageous to have biomarkers which could be used to assess a subject's risk of developing acute coronary syndrome (ACS), or risk of developing ACS-associated impaired vascular function, particularly if the subject is a smoker.

It is primarily to such biomarkers and their use in methods to assess risk of developing such disorders that the present invention is directed.

### BRIEF DESCRIPTION OF THE INVENTION

The present invention is primarily directed to determining the association between genotypes and the subject's risk of developing acute coronary syndrome (ACS). As used herein, ACS includes but is not limited to myocardial infarction, unstable angina, and related acute coronary syndromes.

Thus, according to one aspect there is provided a method of determining a subject's risk of developing ACS comprising analysing a sample from said subject for the presence or absence of one or more polymorphisms selected from the group consisting of:
−1903 A/G in the gene encoding Chymase 1 (CMA1);
−82 A/G in the gene encoding Matrix metalloproteinase 12 (MMP12);
Ser52Ser (223 C/T) in the gene encoding Fibroblast growth factor 2 (FGF2);
Q576R A/G in the gene encoding Interleukin 4 receptor alpha (IL4RA);
HOM T2437C in the gene encoding Heat Shock Protein 70 (HSP 70);
874 A/T in the gene encoding Interferon γ (IFNG);
−589 C/T in the gene encoding Interleukin 4 (IL-4);
−1084 A/G (−1082) in the gene encoding Interleukin 10 (IL-10);
Arg213Gly C/G in the gene encoding Superoxide dismutase 3 (SOD3);
459 C/T Intron I in the gene encoding Macrophage inflammatory protein 1 alpha (MIP1A);
Asn 125 Ser A/G in the gene encoding Cathepsin G;
I249V C/T in the gene encoding Chemokine (CX3C motif) receptor 1 (CX3CR1);
Gly 881 Arg G/C in the gene encoding Caspase (NOD2); or
372 T/C in the gene encoding Tissue inhibitor of metalloproteinase 1 (TIMP1);

wherein the presence or absence of one or more of said polymorphisms is indicative of the subject's risk of developing ACS.

The one or more polymorphisms can be detected directly or by detection of one or more polymorphisms which are in linkage disequilibrium with said one or more polymorphisms.

Linkage disequilibrium (LD) is a phenomenon in genetics whereby two or more mutations or polymorphisms are in such close genetic proximity that they are co-inherited. This means that in genotyping, detection of one polymorphism as present infers the presence of the other. (Reich D E et al; Linkage disequilibrium in the human genome, Nature 2001, 411:199-204.)

The method can additionally comprise analysing a sample from said subject for the presence of one or more further polymorphisms selected from the group consisting of:
−509 C/T in the gene encoding Transforming growth factor β1 (TGFB1);
Thr26Asn A/C in the gene encoding Lymphotoxin α (LTA);
Asp299Gly A/G in the gene encoding Toll-like Receptor 4 (TLR4);
Thr399Ile C/T in the gene encoding TLR4;
−63 T/A in the gene encoding Nuclear factor of kappa light polypeptide gene enhancer in B-cells inhibitor-like 1 (NFKBIL1);
−1630 Ins/Del (AACTT/Del) in the gene encoding Platelet derived growth factor receptor alpha (PDGFRA);
−1607 1G/2G (Del/G) in the gene encoding Matrix metalloproteinase 1 (MMP1);
12 IN 5 C/T in the gene encoding Platelet derived growth factor alpha (PDGFA);
−588 C/T in the gene encoding Glutamate-cysteine ligase modifier subunit (GCLM);
Ile32Val A/G in the gene encoding Olfactory receptor analogue OR13G1 (OR13G1);
Glu288Val A/T (M/S) in the gene encoding alpha 1-antitrypsin (α1-AT);
K469E A/G in the gene encoding Intracellular adhesion molecule 1 (ICAM1);
−23 C/G in the gene encoding HLA-B associated transcript 1 (BAT1);
Glu298Asp G/T in the gene encoding Nitric Oxide synthase 3 (NOS3);
−668 4G/5G in the gene encoding Plasminogen activator inhibitor 1 (PAI-1); or
−A/G in the gene encoding Matrix metalloproteinase 7 (MMP7).

Again, detection of the one or more further polymorphisms may be carried out directly or by detection of polymorphisms in linkage disequilibrium with the one or more further polymorphisms.
The presence of one or more polymorphisms selected from the group consisting of:
the Ser52Ser (223 C/T) CC genotype in the gene encoding FGF2;
the Q576R A/G AA genotype in the gene encoding IL4RA;
the Thr26Asn A/C CC genotype in the gene encoding LTA;
the Hom T2437C CC or CT genotype in the gene encoding HSP70;
the Asp299Gly A/G AG or GG genotype in the gene encoding TLR4;
the Thr399Ile C/T CT or TT genotype in the gene encoding TLR4;
the 874 A/T TT genotype in the gene encoding IFNG;
the −63 T/A AA genotype in the gene encoding NFKBIL1;
the −1630 Ins/Del (AACTT/Del) Ins/Del or Del/Del genotype in the gene encoding PDGFRA;
the −589 C/T CT or TT genotype in the gene encoding IL-4;
the −588 C/T CC genotype in the gene encoding GCLM;
the −1084 A/G GG genotype in the gene encoding IL-10;
the K469E A/G AA genotype in the gene encoding ICAM1;
the −23 C/G GG genotype in the gene encoding BAT1;
the Glu298Asp G/T GG genotype in the gene encoding NOS3;
the Arg213Gly C/G CG or GG genotype in the gene encoding SOD3;
the −668 4G/5G 5G5G genotype in the gene encoding PAI-1;
the −181 A/G GG genotype in the gene encoding MMP7;
the Asn 125 Ser AG or GG genotype in the gene encoding Cathepsin G; or
the 372 T/C TT genotype in the gene encoding TIMP1;
   may be indicative of a decreased risk of developing ACS.

The presence of one or more polymorphisms selected from the group consisting of
the −1903 A/G GG genotype in the gene encoding CMA1;
the −509 C/T CC genotype in the gene encoding TGFB1;
the −82 A/G GG genotype in the gene encoding MMP12;
the Ser52Ser (223 C/T) CT or TT genotype in the gene encoding FGF2;
the Q576R A/G GG genotype in the gene encoding IL4RA;
the Hom T2437C TT genotype in the gene encoding HSP70;
the Asp299Gly A/G AA genotype in the gene encoding TLR4;
the Thr399Ile C/T CC genotype in the gene encoding TLR4;
the −1630 Ins/Del (AACTT/Del) Ins Ins (AACTT AACTT) genotype in the gene encoding PDGFRA;
the −589 C/T CC genotype in the gene encoding IL4;
the −1607 1G/2G (Del/G) Del Del (1G1G) genotype in the gene encoding MMP1;
the 12 IN5 C/T TT genotype in the gene encoding PDGFA;
the −588 C/T CT or TT genotype in the gene encoding GCLM;
the Ile32Val A/G AA genotype in the gene encoding OR13G1;
the Glu288Val A/T (M/S) AT or TT (MS or SS) genotype in the gene encoding α1-AT;
the +459 C/T Intron 1 CT or TT genotype in the gene encoding MIP1A;
the Asn 125 Ser AA genotype in the gene encoding Cathepsin G;
the I249V TT genotype in the gene encoding CX3CR1;
the Gly 881 Arg G/C CC or CG genotype in the gene encoding NOD2; or
the 372 T/C CC genotype in the gene encoding TIMP1; may be indicative of an increased risk of developing ACS.

The methods of the invention are particularly useful in smokers (both current and former).

It will be appreciated that the methods of the invention identify two categories of polymorphisms—namely those associated with a reduced risk of developing ACS (which can be termed “protective polymorphisms”) and those associated with an increased risk of developing ACS (which can be termed “susceptibility polymorphisms”).

Therefore, the present invention further provides a method of assessing a subject's risk of developing ACS, said method comprising:

determining the presence or absence of at least one protective polymorphism associated with a reduced risk of developing ACS; and

in the absence of at least one protective polymorphism, determining the presence or absence of at least one susceptibility polymorphism associated with an increased risk of developing ACS;

wherein the presence of one or more of said protective polymorphisms is indicative of a reduced risk of developing ACS, and the absence of at least one protective polymorphism in combination with the presence of at least one susceptibility polymorphism is indicative of an increased risk of developing ACS.

Preferably, said at least one protective polymorphism is selected from the group consisting of:

The at least one susceptibility polymorphism may be selected from the group consisting of:

In a preferred form of the invention the presence of two or more protective polymorphisms is indicative of a reduced risk of developing ACS.

In a further preferred form of the invention the presence of two or more susceptibility polymorphisms is indicative of an increased risk of developing ACS.

In still a further preferred form of the invention the presence of two or more protective polymorphims irrespective of the presence of one or more susceptibility polymorphisms is indicative of reduced risk of developing ACS.

In another aspect, the invention provides a method of determining a subject's risk of developing ACS, said method comprising obtaining the result of one or more genetic tests of a sample from said subject, and analysing the result for the presence or absence of one or more polymorphisms selected from the group consisting of:
−1903 A/G in the gene encoding Chymase 1 (CMA1);
−82 A/G in the gene encoding Matrix metalloproteinase 12 (MMP12);
Ser52Ser (223 C/T) in the gene encoding Fibroblast growth factor 2 (FGF2);
Q576R A/G in the gene encoding Interleukin 4 receptor alpha (IL4RA);
HOM T2437C in the gene encoding Heat Shock Protein 70 (HSP 70);
874 A/T in the gene encoding Interferon γ (IFNG);
−589 C/T in the gene encoding Interleukin 4 (IL-4);
−1084 A/G (−1082) in the gene encoding Interleukin 10 (IL-10);
Arg213Gly C/G in the gene encoding Superoxide dismutase 3 (SOD3);
459 C/T Intron I in the gene encoding Macrophage inflammatory protein 1 alpha (MIP1A);
Asn 125 Ser A/G in the gene encoding Cathepsin G;
I249V C/T in the gene encoding Chemokine (CX3C motif) receptor 1 (CX3CR1);
Gly 881 Arg G/C in the gene encoding Caspase (NOD2); or
372 T/C in the gene encoding Tissue inhibitor of metalloproteinase 1 (TIMP1); or one or more polymorphisms in linkage disequilibrium with any one or more of these polymorphisms;

wherein a result indicating the presence or absence of one or more of said polymorphisms is indicative of the subject's risk of developing ACS.

In a further aspect there is provided a method of determining a subject's risk of developing ACS comprising the analysis of two or more polymorphisms selected from the group consisting of:
−1903 A/G in the gene encoding Chymase 1 (CMA1);
−82 A/G in the gene encoding Matrix metalloproteinase 12 (MMP12);
Ser52Ser (223 C/T) in the gene encoding Fibroblast growth factor 2 (FGF2);
Q576R A/G in the gene encoding Interleukin 4 receptor alpha (IL4RA);
HOM T2437C in the gene encoding Heat Shock Protein 70 (HSP 70);
874 A/T in the gene encoding Interferon γ (IFNG);
−589 C/T in the gene encoding Interleukin 4 (IL-4);
−1084 A/G (−1082) in the gene encoding Interleukin 10 (IL-10);
Arg213Gly C/G in the gene encoding Superoxide dismutase 3 (SOD3);
459 C/T Intron I in the gene encoding Macrophage inflammatory protein 1 alpha (MIP1A);
−509 C/T in the gene encoding Transforming growth factor β1 (TGFB1);
Thr26Asn A/C in the gene encoding Lymphotoxin a (LTA);
Asp299Gly A/G in the gene encoding Toll-like Receptor 4 (TLR4);
Thr399Ile C/T in the gene encoding TLR4;
−63 T/A in the gene encoding Nuclear factor of kappa light polypeptide gene enhancer in B-cells inhibitor-like 1 (NFKBIL1);
−1630 Ins/Del (AACTT/Del) in the gene encoding Platelet derived growth factor receptor alpha (PDGFRA);
−1607 1G/2G (Del/G) in the gene encoding Matrix metalloproteinase 1 (MMP1);
12 IN 5 C/T in the gene encoding Platelet derived growth factor alpha (PDGFA);
−588 C/T in the gene encoding Glutamate-cysteine ligase modifier subunit (GCLM);
Ile32Val A/G in the gene encoding Olfactory receptor analogue OR13G1 (OR13G1);
Glu288Val A/T (M/S) in the gene encoding alpha 1-antitrypsin (α1-AT);
K469E A/G in the gene encoding Intracellular adhesion molecule 1 (ICAM1);
−23 C/G in the gene encoding HLA-B associated transcript 1 (BAT1);
Glu298Asp G/T in the gene encoding Nitric Oxide synthase 3 (NOS3);
−668 4G/5G in the gene encoding Plasminogen activator inhibitor 1 (PAI-1); or
−181 A/G in the gene encoding Matrix metalloproteinase 7 (MMP7);
Asn 125 Ser A/G in the gene encoding Cathepsin G;
I249V C/T in the gene encoding Chemokine (CX3C motif) receptor 1 (CX3CR1);
Gly 881 Arg G/C in the gene encoding Caspase (NOD2); or
372 T/C in the gene encoding Tissue inhibitor of metalloproteinase 1 (TIMP1); or one or more polymorphisms in linkage disequilibrium with any one or more of these polymorphisms.

In various embodiments, any one or more of the above methods comprises the step of analysing the amino acid present at a position mapping to codon 576 of the gene encoding IL4RA.

The presence of glutamine at said position is indicative of a reduced risk of developing ACS.

The presence of arginine at said position is indicative of an increased risk of developing ACS.

In various embodiments, any one or more of the above methods comprises the step of analysing the amino acid present at a position mapping to codon 26 of the gene encoding LTA.

The presence of threonine at said position is indicative of a decreased risk of developing ACS.

The presence of asparagine at said position is indicative of an increased risk of developing ACS.

In various embodiments, any one or more of the above methods comprises the step of analysing the amino acid present at a position mapping to codon 299 of the gene encoding TLR4.

The presence of glycine at said position is indicative of a decreased risk of developing ACS.

The presence of aspartate at said position is indicative of an increased risk of developing ACS.

In various embodiments, any one or more of the above methods comprises the step of analysing the amino acid present at a position mapping to codon 399 of the gene encoding TLR4.

The presence of isoleucine at said position is indicative of a decreased risk of developing ACS.

The presence of threonine at said position may be indicative of an increased risk of developing ACS.

In various embodiments, any one or more of the above methods comprises the step of analysing the amino acid present at a position mapping to codon 132 of the gene encoding OR13G1.

The presence of isoleucine at said position is indicative of an increased risk of developing ACS.

In various embodiments, any one or more of the above methods comprises the step of analysing the amino acid present at a position mapping to codon 288 of the gene encoding α1-AT.

The presence of glutamate at said position is indicative of an increased risk of developing ACS.

In various embodiments, any one or more of the above methods comprises the step of analysing the amino acid present at a position mapping to codon 496 of the gene encoding ICAM1.

The presence of lysine at said position is indicative of a decreased risk of developing ACS.

In various embodiments, any one or more of the above methods comprises the step of analysing the amino acid present at a position mapping to codon 298 of the gene encoding NOS3.

The presence of glutamate at said position is indicative of a decreased risk of developing ACS.

The presence of aspartate at said position is indicative of an increased risk of developing ACS.

In various embodiments, any one or more of the above methods comprises the step of analysing the amino acid present at a position mapping to codon 213 of the gene encoding SOD3.

The presence of glycine at said position is indicative of a decreased risk of developing ACS.

In various embodiments, any one or more of the above methods comprises the step of analysing the amino acid present at a position mapping to codon 125 of the gene encoding Cathespin G.

The presence of serine at said position is indicative of a decreased risk of developing ACS.

The presence of asparagine at said position is indicative of an increased risk of developing ACS.

In various embodiments, any one or more of the above methods comprises the step of analysing the amino acid present at a position mapping to codon 249 of the gene encoding CX3CR1.

The presence of isoleucine at said position is indicative of an increased risk of developing ACS.

In various embodiments, any one or more of the above methods comprises the step of analysing the amino acid present at a position mapping to codon 881 of the gene encoding NOD2.

The presence of arginine at said position is indicative of an increased risk of developing ACS.

In a preferred form of the invention the methods as described herein are performed in conjunction with an analysis of one or more risk factors, including one or more epidemiological risk factors, associated with a risk of developing ACS. Such epidemiological risk factors include but are not limited to smoking or exposure to tobacco smoke, age, sex, and familial history of ACS.

In a further aspect, the invention provides for the use of at least one polymorphism in the assessment of a subject's risk of developing ACS, wherein said at least one polymorphism is selected from the group consisting of:
−1903 A/G in the gene encoding Chymase 1 (CMA1);
−82 A/G in the gene encoding Matrix metalloproteinase 12 (MMP12);
Ser52Ser (223 C/T) in the gene encoding Fibroblast growth factor 2 (FGF2);
Q576R A/G in the gene encoding Interleukin 4 receptor alpha (IL4RA);
HOM T2437C in the gene encoding Heat Shock Protein 70 (HSP 70);
874 A/T in the gene encoding Interferon γ (IFNG);
−589 C/T in the gene encoding Interleukin 4 (IL-4);
−1084 A/G (−1082) in the gene encoding Interleukin 10 (IL-10);
Arg213Gly C/G in the gene encoding Superoxide dismutase 3 (SOD3);
459 C/T Intron I in the gene encoding Macrophage inflammatory protein 1 alpha (MIP1A);
Asn 125 Ser A/G in the gene encoding Cathepsin G;
I249V C/T in the gene encoding Chemokine (CX3C motif) receptor 1 (CX3CR1);
Gly 881 Arg G/C in the gene encoding Caspase (NOD2); or
372 T/C in the gene encoding Tissue inhibitor of metalloproteinase 1 (TIMP1); or one or more polymorphisms in linkage disequilibrium with any one of said polymorphisms.

Optionally, said use may be in conjunction with the use of at least one further polymorphism selected from the group consisting of:
−509 C/T in the gene encoding Transforming growth factor β1 (TGFB1);
Thr26Asn A/C in the gene encoding Lymphotoxin α (LTA);
Asp299Gly A/G in the gene encoding Toll-like Receptor 4 (TLR4);
Thr399Ile C/T in the gene encoding TLR4;
−63 T/A in the gene encoding Nuclear factor of kappa light polypeptide gene enhancer in B-cells inhibitor-like 1 (NFKBIL1);
−1630 Ins/Del (AACTT/Del) in the gene encoding Platelet derived growth factor receptor alpha (PDGFRA);
−1607 1G/2G (Del/G) in the gene encoding Matrix metalloproteinase 1 (MMP1);
12 IN 5 C/T in the gene encoding Platelet derived growth factor alpha (PDGFA);
−588 C/T in the gene encoding Glutamate-cysteine ligase modifier subunit (GCLM);
Ile32Val A/G in the gene encoding Olfactory receptor analogue OR13G1 (OR13G1);
Glu288Val A/T (M/S) in the gene encoding alpha 1-antitrypsin (α1-AT);
K469E A/G in the gene encoding Intracellular adhesion molecule 1 (ICAM1);
−23 C/G in the gene encoding HLA-B associated transcript 1 (BAT1);
Glu298Asp G/T in the gene encoding Nitric Oxide synthase 3 (NOS3);
−668 4G/5G in the gene encoding Plasminogen activator inhibitor 1 (PAI-1);
−181 A/G in the gene encoding Matrix metalloproteinase 7 (MMP7);
or one or more polymorphisms which are in linkage disequilibrium with any one or more of these polymorphisms.

In another aspect the invention provides a set of nucleotide probes and/or primers for use in the preferred methods of the invention herein described. Preferably, the nucleotide probes and/or primers are those which span, or are able to be used to span, the polymorphic regions of the genes. Also provided are one or more nucleotide probes and/or primers comprising the sequence of any one of the probes and/or primers herein described, including any one comprising the sequence of any one of SEQ.ID.NO. 1 to 124.

In yet a further aspect, the invention provides a nucleic acid microarray for use in the methods of the invention, which microarray comprises a substrate presenting nucleic acid sequences capable of hybridizing to nucleic acid sequences which encode one or more of the susceptibility or protective polymorphisms described herein or sequences complimentary thereto.

In another aspect, the invention provides an antibody microarray for use in the methods of the invention, which microarray comprises a substrate presenting antibodies capable of binding to a product of expression of a gene the expression of which is upregulated or downregulated when associated with a susceptibility or protective polymorphism as described herein.

In a further aspect the present invention provides a method treating a subject having an increased risk of developing ACS comprising the step of replicating, genotypically or phenotypically, the presence and/or functional effect of a protective polymorphism in said subject.

In yet a further aspect, the present invention provides a method of treating a subject having an increased risk of developing ACS, said subject having a detectable susceptibility polymorphism which either upregulates or downregulates expression of a gene such that the physiologically active concentration of the expressed gene product is outside a range which is normal for the age and sex of the subject, said method comprising the step of restoring the physiologically active concentration of said product of gene expression to be within a range which is normal for the age and sex of the subject.

In a further aspect the present invention provides a method of treating a subject having an increased risk of developing ACS due to the presence of a polymorphism predictive of susceptibility to ACS comprising the step of reversing, genotypically or phenotypically, the functional effect of said polymorphism in said subject.

In yet still a further aspect the present invention provides a method of treating a subject having an increased risk of developing ACS and for whom the presence of the GG genotype at the −82 A/G polymorphism in the promoter of the gene encoding MMP12 has been determined, said method comprising administering to said subject an agent capable of modulating MMP12 activity in said subject.

In one embodiment, said agent is an agent capable of increasing expression of or the activity of one or more tissue inhibitors of metalloproteinases (TIMPs), preferably the expression or activity of one or more of TIMP1, TIMP2, TIMP3, or TIMP4. In a further embodiment, said agent is an agent capable of reducing expression of or the activity of one or more membrane bound MMPs. In still a father embodiment, said agent is a MMP inhibitor, preferably said MMP inhibitor is selected from the group comprising 4,5-dihydroxyanthaquinone-2-carboxylic acid (AQCA), anthraquinyl-mercaptoethyamine, anthraquinyl-alanine hydroxamate, and derivatives thereof.

In yet still a further aspect the present invention provides a method of treating a subject having an increased risk of developing ACS and for whom the presence of the CC genotype at the 372 T/C polymorphism in the gene encoding TIMP1 has been determined, said method comprising administering to said subject an agent capable of modulating TIMP1 activity in said subject.

In one embodiment, said agent is an agent capable of increasing expression of or the activity of TIMP1.

In yet a further aspect, the present invention provides a method for screening for compounds that modulate the expression and/or activity of a gene, the expression of which is upregulated or downregulated when associated with a susceptibility or protective polymorphism (as compared to the level of expression of said gene when not associated with said polymorphism), said method comprising the steps of:

contacting a candidate compound with a cell comprising a susceptibility or protective polymorphism which has been determined to be associated with the upregulation or downregulation of expression of a gene; and

measuring the expression of said gene following contact with said candidate compound,

wherein a change in the level of expression after the contacting step as compared to before the contacting step is indicative of the ability of the compound to modulate the expression and/or activity of said gene.

Preferably, said cell is a human vascular cell, more preferably a human vascular epithelial cell, which has been pre-screened to confirm the presence of said polymorphism.

Preferably, said cell comprises a susceptibility polymorphism associated with upregulation of expression of said gene and said screening is for candidate compounds which downregulate expression of said gene.

Alternatively, said cell comprises a susceptibility polymorphism associated with downregulation of expression of said gene and said screening is for candidate compounds which upregulate expression of said gene.

In another embodiment, said cell comprises a protective polymorphism associated with upregulation of expression of said gene and said screening is for candidate compounds which further upregulate expression of said gene.

Alternatively, said cell comprises a protective polymorphism associated with downregulation of expression of said gene and said screening is for candidate compounds which further downregulate expression of said gene.

In another aspect, the present invention provides a method for screening for compounds that modulate the expression and/or activity of a gene, the expression of which is upregulated or downregulated when associated with a susceptibility or protective polymorphism, said method comprising the steps of:

contacting a candidate compound with a cell comprising a gene, the expression of which is upregulated or downregulated when associated with a susceptibility or protective polymorphism but which in said cell the expression of which is neither upregulated nor downregulated; and

measuring the expression of said gene following contact with said candidate compound,

wherein a change in the level of expression after the contacting step as compared to before the contacting step is indicative of the ability of the compound to modulate the expression and/or activity of said gene.

Preferably, expression of the gene is downregulated when associated with a susceptibility polymorphism once said screening is for candidate compounds which in said cell, upregulate expression of said gene.

Preferably, said cell is a human vascular cell, more preferably a human vascular epithelial cell, which has been pre-screened to confirm the presence, and baseline level of expression, of said gene.

Alternatively, expression of the gene is upregulated when associated with a susceptibility polymorphism and said screening is for candidate compounds which, in said cell, downregulate expression of said gene.

In another embodiment, expression of the gene is upregulated when associated with a protective polymorphism and said screening is for compounds which, in said cell, upregulate expression of said gene.

Alternatively, expression of the gene is downregulated when associated with a protective polymorphism and said screening is for compounds which, in said cell, downregulate expression of said gene.

In yet a further aspect, the present invention provides a method of assessing the likely responsiveness of a subject at risk of developing or suffering from ACS to a prophylactic or therapeutic treatment, which treatment involves restoring the physiologically active concentration of a product of gene expression to be within a range which is normal for the age and sex of the subject, which method comprises detecting in said subject the presence or absence of a susceptibility polymorphism which when present either upregulates or downregulates expression of said gene such that the physiological active concentration of the expressed gene product is outside said normal range, wherein the detection of the presence of said polymorphism is indicative of the subject likely responding to said treatment.

In a further aspect, the present invention provides a kit for assessing a subject's risk of developing ACS, said kit comprising a means of analysing a sample from said subject for the presence or absence of one or more polymorphisms disclosed herein.

### BRIEF DESCRIPTION OF THE DRAWINGS

FIG. 1: depicts a graph showing the frequency of ACS plotted against SNP score derived from the 11 SNP panel.

FIG. 2: depicts a graph showing the frequency of ACS plotted against the SNP score derived from the 15 SNP panel.

FIG. 3 depicts a graph showing the log odds of having ACS according to SNP score derived from the 11 SNP panel.

FIG. 4 depicts a graph showing the frequency of ACS against SNP score derived from the substituted 11 SNP panel.

### DESCRIPTION OF THE PREFERRED EMBODIMENTS

Using case-control studies the frequencies of several genetic variants (polymorphisms) of candidate genes in smokers who have developed ACS and blood donor controls have been compared. The majority of these candidate genes have confirmed (or likely) functional effects on gene expression or protein function. Specifically, the frequencies of polymorphisms between blood donor controls, resistant smokers and those with ACS have been compared.

In one embodiment described herein 20 susceptibility genetic polymorphisms and 20 protective genetic polymorphisms are identified. These are as follows:

| Gene | Polymorphism | Genotype | Phenotype |
|---|---|---|---|
| CMA1 | −1903 A/G | GG | susceptibility |
| TGFB1 | −509 C/T | CC | susceptibility |
| MMP12 | −82 A/G | GG | susceptibility |
| FGF2 | Ser52Ser 223 C/T | CT/TT | susceptibility |
| | | CC | protective |
| IL4RA | Q576R A/G | GG | susceptibility |
| | | AA | protective |
| LTA | Thr26Asn A/C | CC | protective |
| HSP70 | Hom T2437C | CC/CT | protective |
| | | TT | susceptibility |
| TLR4 | Asp299Gly A/G | AG/GG | protective |
| | | AA | susceptibility |
| TLR4 | Thr399Ile C/T | CT/TT | protective |
| | | CC | susceptibility |
| IFNG | 874 A/T | TT | protective |
| NFKBIL1 | −63 T/A | AA | protective |
| PDGFRA | −1630 I/D, AACTT/Del | I/Del, | protective |
| | | Del/Del | susceptibility |
| | | II | |
| IL4 | −589 C/T | CT/TT | protective |
| | | CC | susceptibility |
| MMP1 | −1607 1G/2G, Del/G | Del.Del | susceptibility |
| | | ie 1G1G | |
| PDGFA | 12 IN5 C/T | TT | susceptibility |
| GCLM | −588 C/T | CT/TT | susceptibility |
| | | CC | protective |
| OR13G1 | Ile132Val A/G | AA | susceptibility |
| IL-10 | −1084 A/G (−1082) | GG | protective |
| α1-AT | Glu288Val A/T (M/S) | AT/TT | susceptibility |
| S allele | | MS/SS | |
| ICAM1 | K469E A/G | AA | protective |
| BAT1 | −23 C/G | GG | protective |
| NOS3 | Glu298Asp G/T | GG | protective |
| SOD3 | Arg213Gly C/G | CG/GG | protective |
| PAI-1 | −668 4G/5G | 5G5G | protective |
| MIP1A | +459 C/T Intron 1 | CT/TT | susceptibility |
| MMP7 | −181 A/G | GG | protective |
| Cathepsin G | Asn 125Ser | AG/GG | protective |
| | | AA | susceptibility |
| CX3CR1 | I249V | TT | susceptibility |
| NOD2 | Gly 881 Arg G/C | CC/CG | susceptibility |
| TIMP1 | 372 T/C | TT | protective |
| | | CC | susceptibility |

A susceptibility genetic polymorphism is one which, when present, is indicative of an increased risk of developing ACS. In contrast, a protective genetic polymorphism is one which, when present, is indicative of a reduced risk of developing ACS.

As used herein, the phrase “risk of developing ACS” means the likelihood that a subject to whom the risk applies will develop ACS, and includes predisposition to, and potential onset of the disease. Accordingly, the phrase “increased risk of developing ACS” means that a subject having such an increased risk possesses an hereditary inclination or tendency to develop ACS. This does not mean that such a person will actually develop ACS at any time, merely that he or she has a greater likelihood of developing ACS compared to the general population of individuals that either does not possess a polymorphism associated with increased ACS or does possess a polymorphism associated with decreased ACS risk. Subjects with an increased risk of developing ACS include those with a predisposition to ACS, such as a tendency or predilection regardless of their vascular function at the time of assessment, for example, a subject who is genetically inclined to ACS but who has normal vascular function, those at potential risk, including subjects with a tendency to mildly reduced vascular function who are likely to go on to suffer ACS if they keep smoking, and subjects with potential onset of ACS, who have a tendency to poor vascular function consistent with ACS at the time of assessment.

Similarly, the phrase “decreased risk of developing ACS” means that a subject having such a decreased risk possesses an hereditary disinclination or reduced tendency to develop ACS. This does not mean that such a person will not develop ACS at any time, merely that he or she has a decreased likelihood of developing ACS compared to the general population of individuals that either does possess one or more polymorphisms associated with increased ACS, or does not possess a polymorphism associated with decreased ACS.

It will be understood that in the context of the present invention the term “polymorphism” means the occurrence together in the same population at a rate greater than that attributable to random mutation (usually greater than 1%) of two or more alternate forms (such as alleles or genetic markers) of a chromosomal locus that differ in nucleotide sequence or have variable numbers of repeated nucleotide units. See www.ornl.gov/sci/techresources/Human_Genome/publicat/97pr/09gloss.html#p. Accordingly, the term “polymorphisms” is used herein contemplates genetic variations, including single nucleotide substitutions, insertions and deletions of nucleotides, repetitive sequences (such as microsatellites), and the total or partial absence of genes (eg. null mutations). As used herein, the term “polymorphisms” also includes genotypes and haplotypes. A genotype is the genetic composition at a specific locus or set of loci. A haplotype is a set of closely linked genetic markers present on one chromosome which are not easily separable by recombination, tend to be inherited together, and may be in linkage disequilibrium. A haplotype can be identified by patterns of polymorphisms such as SNPs. Similarly, the term “single nucleotide polymorphism” or “SNP” in the context of the present invention includes single base nucleotide substitutions and short deletion and insertion polymorphisms.

A reduced or increased risk of a subject developing ACS may be diagnosed by analysing a sample from said subject for the presence of a polymorphism selected from the group consisting of:
−1903 A/G in the gene encoding Chymase 1 (CMA1);
−82 A/G in the gene encoding Matrix metalloproteinase 12 (MMP12);
Ser52Ser (223 C/T) in the gene encoding Fibroblast growth factor 2 (FGF2);
Q576R A/G in the gene encoding Interleukin 4 receptor alpha (IL4RA);
HOM T2437C in the gene encoding Heat Shock Protein 70 (HSP 70);
874 A/T in the gene encoding Interferon γ (IFNG);
−589 C/T in the gene encoding Interleukin 4 (IL-4);
−1084 A/G (−1082) in the gene encoding Interleukin 10 (IL-10);
Arg213Gly C/G in the gene encoding Superoxide dismutase 3 (SOD3);
459 C/T Intron I in the gene encoding Macrophage inflammatory protein 1 alpha (MIP1A);
Asn 125 Ser A/G in the gene encoding Cathepsin G;
I249V C/T in the gene encoding Chemokine (CX3C motif) receptor 1 (CX3CR1);
Gly 881 Arg G/C in the gene encoding Caspase (NOD2); or

372 T/C in the gene encoding Tissue inhibitor of metalloproteinase 1 (TIMP1); or one or more polymorphisms which are in linkage disequilibrium with any one or more of the above group.

These polymorphisms can also be analysed in combinations of two or more, or in combination with other polymorphisms indicative of a subject's risk of developing ACS, inclusive of the remaining polymorphisms listed above.

Assays which involve combinations of polymorphisms, including those amenable to high throughput, such as those utilising microarrays, are preferred.

Statistical analyses, particularly of the combined effects of these polymorphisms, show that the genetic assays of the present invention can be used to determine the risk quotient of any subject (including smokers) and in particular to identify subjects at greater risk of developing ACS. Such combined analysis can be of combinations of susceptibility polymorphisms only, of protective polymorphisms only, or of combinations of both. Analysis can also be step-wise, with analysis of the presence or absence of protective polymorphisms occurring first and then with analysis of susceptibility polymorphisms proceeding only where no protective polymorphisms are present.

Thus, through systematic analysis of the frequency of these polymorphisms in well defined groups of subjects including smokers and non-smokers as described herein, it is possible to implicate certain genes and proteins in the development of ACS and improve the ability to identify which subjects are at increased risk of developing ACS-related impaired vascular function and ACS for predictive purposes.

The present results show that the minority of smokers who develop ACS do so because they have one or more of the susceptibility polymorphisms and few or none of the protective polymorphisms defined herein. It is thought that the presence of one or more susceptibility polymorphisms, together with the damaging irritant and oxidant effects of smoking, combine to make this group of smokers highly susceptible to developing ACS. Additional risk factors, such as familial history, age, weight, pack years, etc., will also have an impact on the risk profile of a subject, and can be assessed in combination with the genetic analyses described herein.

The one or more polymorphisms can be detected directly or by detection of one or more polymorphisms which are in linkage disequilibrium with said one or more polymorphisms. As discussed above, linkage disequilibrium is a phenomenon in genetics whereby two or more mutations or polymorphisms are in such close genetic proximity that they are co-inherited. This means that in genotyping, detection of one polymorphism as present infers the presence of the other. (Reich D E et al; Linkage disequilibrium in the human genome, Nature 2001, 411:199-204.)

Examples of polymorphisms described herein that have been reported to be in linkage disequilibrium are presented herein, and include the MMP12-82 A/G and MMP1−1607 1G/2G (Del/G) polymorphisms, the LTA Thr26Asn A/C and NFKBIL1-63 T/A polymorphsisms, and the TLR4 Asp299Gly A/G and Thr399Ile C/T polymorphisms as shown herein in Example 2 and Table 33.

It will be apparent that polymorphsisms in linkage disequilibrium with one or more other polymorphism associated with increased or decreased risk of developing ACS will also provide utility as biomarkers for risk of developing ACS. The data presented herein shows that the frequency for SNPs in linkage disequilibrium is very similar. Accordingly, these genetically linked SNPs can be utilized in combined polymorphism analyses to derive a level of risk comparable to that calculated from the original SNP. An example of such an analysis in which SNPs in LD are substituted one for the other is presented herein in Example 2.

It will therefore be apparent that one or more polymorphisms in linkage disequilibrium with the polymorphisms specified herein can be identified, for example, using public data bases. Examples of such polymorphisms reported to be in linkage disequilibrium with the polymorphisms specified herein are presented herein in Table 35.

It will also be apparent that frequently a variety of nomenclatures may exist for any given polymorphism. For example, the polymorphism referred to herein as Arg 213 Gly in the gene encoding SOD3 is believed to have been referred to variously as Arg 312 Gln, +760 G/C, and Arg 231 Gly (rs1799895). When referring to a susceptibility or protective polymorphism as herein described, such alternative nomenclatures are also contemplated by the present invention.

The methods of the invention are primarily directed to the detection and identification of the above polymorphisms associated with ACS. These polymorphisms are typically single nucleotide polymorphisms. In general terms, a single nucleotide polymorphism (SNP) is a single base change or point mutation resulting in genetic variation between individuals. SNPs occur in the human genome approximately once every 100 to 300 bases, and can occur in coding or non-coding regions. Due to the redundancy of the genetic code, a SNP in the coding region may or may not change the amino acid sequence of a protein product. A SNP in a non-coding region can, for example, alter gene expression by, for example, modifying control regions such as promoters, transcription factor binding sites, processing sites, ribosomal binding sites, and affect gene transcription, processing, and translation.

SNPs can facilitate large-scale association genetics studies, and there has recently been great interest in SNP discovery and detection. SNPs show great promise as markers for a number of phenotypic traits (including latent traits), such as for example, disease propensity and severity, wellness propensity, and drug responsiveness including, for example, susceptibility to adverse drug reactions. Knowledge of the association of a particular SNP with a phenotypic trait, coupled with the knowledge of whether an individual has said particular SNP, can enable the targeting of diagnostic, preventative and therapeutic applications to allow better disease management, to enhance understanding of disease states and to ultimately facilitate the discovery of more effective treatments, such as personalised treatment regimens.

Indeed, a number of databases have been constructed of known SNPs, and for some such SNPs, the biological effect associated with a SNP. For example, the NCBI SNP database “dbSNP” is incorporated into NCBI's Entrez system and can be queried using the same approach as the other Entrez databases such as PubMed and GenBank. This database has records for over 1.5 million SNPs mapped onto the human genome sequence. Each dbSNP entry includes the sequence context of the polymorphism (i.e., the surrounding sequence), the occurrence frequency of the polymorphism (by population or individual), and the experimental method(s), protocols, and conditions used to assay the variation, and can include information associating a SNP with a particular phenotypic trait.

At least in part because of the potential impact on health and wellness, there has been and continues to be a great deal of effort to develop methods that reliably and rapidly identify SNPs. This is no trivial task, at least in part because of the complexity of human genomic DNA, with a haploid genome of 3×10⁹ base pairs, and the associated sensitivity and discriminatory requirements.

Genotyping approaches to detect SNPs well-known in the art include DNA sequencing, methods that require allele specific hybridization of primers or probes, allele specific incorporation of nucleotides to primers bound close to or adjacent to the polymorphisms (often referred to as “single base extension”, or “minisequencing”), allele-specific ligation (joining) of oligonucleotides (ligation chain reaction or ligation padlock probes), allele-specific cleavage of oligonucleotides or PCR products by restriction enzymes (restriction fragment length polymorphisms analysis or RFLP) or chemical or other agents, resolution of allele-dependent differences in electrophoretic or chromatographic mobilities, by structure specific enzymes including invasive structure specific enzymes, or mass spectrometry. Analysis of amino acid variation is also possible where the SNP lies in a coding region and results in an amino acid change.

DNA sequencing allows the direct determination and identification of SNPs. The benefits in specificity and accuracy are generally outweighed for screening purposes by the difficulties inherent in whole genome, or even targeted subgenome, sequencing.

Mini-sequencing involves allowing a primer to hybridize to the DNA sequence adjacent to the SNP site on the test sample under investigation. The primer is extended by one nucleotide using all four differentially tagged fluorescent dideoxynucleotides (A, C, G, or T), and a DNA polymerase. Only one of the four nucleotides (homozygous case) or two of the four nucleotides (heterozygous case) is incorporated. The base that is incorporated is complementary to the nucleotide at the SNP position.

A number of methods currently used for SNP detection involve site-specific and/or allele-specific hybridisation. These methods are largely reliant on the discriminatory binding of oligonucleotides to target sequences containing the SNP of interest. The techniques of Affymetrix (Santa Clara, Calif.) and Nanogen Inc. (San Diego, Calif.) are particularly well-known, and utilize the fact that DNA duplexes containing single base mismatches are much less stable than duplexes that are perfectly base-paired. The presence of a matched duplex is detected by fluorescence.

The majority of methods to detect or identify SNPs by site-specific hybridisation require target amplification by methods such as PCR to increase sensitivity and specificity (see, for example U.S. Pat. No. 5,679,524, PCT publication WO 98/59066, PCT publication WO 95/12607). US Application 20050059030 (incorporated herein in its entirety) describes a method for detecting a single nucleotide polymorphism in total human DNA without prior amplification or complexity, reduction to selectively enrich for the target sequence, and without the aid of any enzymatic reaction. The method utilises a single-step hybridization involving two hybridization events: hybridization of a first portion of the target sequence to a capture probe, and hybridization of a second portion of said target sequence to a detection probe. Both hybridization events happen in the same reaction, and the order in which hybridisation occurs is not critical.

US Application 20050042608 (incorporated herein in its entirety) describes a modification of the method of electrochemical detection of nucleic acid hybridization of Thorp et al. (U.S. Pat. No. 5,871,918). Briefly, capture probes are designed, each of which has a different SNP base and a sequence of probe bases on each side of the SNP base. The probe bases are complementary to the corresponding target sequence adjacent to the SNP site. Each capture probe is immobilized on a different electrode having a non-conductive outer layer on a conductive working surface of a substrate. The extent of hybridization between each capture probe and the nucleic acid target is detected by detecting the oxidation-reduction reaction at each electrode, utilizing a transition metal complex. These differences in the oxidation rates at the different electrodes are used to determine whether the selected nucleic acid target has a single nucleotide polymorphism at the selected SNP site.

The technique of Lynx Therapeutics (Hayward, Calif.) using MEGATYPE™ technology can genotype very large numbers of SNPs simultaneously from small or large pools of genomic material. This technology uses fluorescently labeled probes and compares the collected genomes of two populations, enabling detection and recovery of DNA fragments spanning SNPs that distinguish the two populations, without requiring prior SNP mapping or knowledge.

A number of other methods for detecting and identifying SNPs exist. These include the use of mass spectrometry, for example, to measure probes that hybridize to the SNP. This technique varies in how rapidly it can be performed, from a few samples per day to a high throughput of 40,000 SNPs per day, using mass code tags. A preferred example is the use of mass spectrometric determination of a nucleic acid sequence which comprises the polymorphisms of the invention, for example, which includes the promoter of the COX2 gene or a complementary sequence. Such mass spectrometric methods are known to those skilled in the art, and the genotyping methods of the invention are amenable to adaptation for the mass spectrometric detection of the polymorphisms of the invention, for example, the COX2 promoter polymorphisms of the invention.

SNPs can also be determined by ligation-bit analysis. This analysis requires two primers that hybridize to a target with a one nucleotide gap between the primers. Each of the four nucleotides is added to a separate reaction mixture containing DNA polymerase, ligase, target DNA and the primers. The polymerase adds a nucleotide to the 3′ end of the first primer that is complementary to the SNP, and the ligase then ligates the two adjacent primers together. Upon heating of the sample, if ligation has occurred, the now larger primer will remain hybridized and a signal, for example, fluorescence, can be detected. A further discussion of these methods can be found in U.S. Pat. Nos. 5,919,626; 5,945,283; 5,242,794; and 5,952,174.

U.S. Pat. No. 6,821,733 (incorporated herein in its entirety) describes methods to detect differences in the sequence of two nucleic acid molecules that includes the steps of: contacting two nucleic acids under conditions that allow the formation of a four-way complex and branch migration; contacting the four-way complex with a tracer molecule and a detection molecule under conditions in which the detection molecule is capable of binding the tracer molecule or the four-way complex; and determining binding of the tracer molecule to the detection molecule before and after exposure to the four-way complex. Competition of the four-way complex with the tracer molecule for binding to the detection molecule indicates a difference between the two nucleic acids.

Protein- and proteomics-based approaches are also suitable for polymorphism detection and analysis. Polymorphisms which result in or are associated with variation in expressed proteins can be detected directly by analysing said proteins. This typically requires separation of the various proteins within a sample, by, for example, gel electrophoresis or HPLC, and identification of said proteins or peptides derived therefrom, for example by NMR or protein sequencing such as chemical sequencing or more prevalently mass spectrometry. Proteomic methodologies are well known in the art, and have great potential for automation. For example, integrated systems, such as the ProteomIQ™ system from Proteome Systems, provide high throughput platforms for proteome analysis combining sample preparation, protein separation, image acquisition and analysis, protein processing, mass spectrometry and bioinformatics technologies.

The majority of proteomic methods of protein identification utilise mass spectrometry, including ion trap mass spectrometry, liquid chromatography (LC) and LC/MSn mass spectrometry, gas chromatography (GC) mass spectroscopy, Fourier transform-ion cyclotron resonance-mass spectrometer (FT-MS), MALDI-TOF mass spectrometry, and ESI mass spectrometry, and their derivatives. Mass spectrometric methods are also useful in the determination of post-translational modification of proteins, such as phosphorylation or glycosylation, and thus have utility in determining polymorphisms that result in or are associated with variation in post-translational modifications of proteins.

Associated technologies are also well known, and include, for example, protein processing devices such as the “Chemical Inkjet Printer” comprising piezoelectric printing technology that allows in situ enzymatic or chemical digestion of protein samples electroblotted from 2-D PAGE gels to membranes by jetting the enzyme or chemical directly onto the selected protein spots. After in-situ digestion and incubation of the proteins, the membrane can be placed directly into the mass spectrometer for peptide analysis.

A large number of methods reliant on the conformational variability of nucleic acids have been developed to detect SNPs.

For example, Single Strand Conformational Polymorphism (SSCP, Orita et al., PNAS 1989 86:2766-2770) is a method reliant on the ability of single-stranded nucleic acids to form secondary structure in solution under certain conditions. The secondary structure depends on the base composition and can be altered by a single nucleotide substitution, causing differences in electrophoretic mobility under nondenaturing conditions. The various polymorphs are typically detected by autoradiography when radioactively labelled, by silver staining of bands, by hybridisation with detectably labelled probe fragments or the use of fluorescent PCR primers which are subsequently detected, for example by an automated DNA sequencer.

Modifications of SSCP are well known in the art, and include the use of differing gel running conditions, such as for example differing temperature, or the addition of additives, and different gel matrices. Other variations on SSCP are well known to the skilled artisan, including, RNA-SSCP, restriction endonuclease fingerprinting-SSCP, dideoxy fingerprinting (a hybrid between dideoxy sequencing and SSCP), bi-directional dideoxy fingerprinting (in which the dideoxy termination reaction is performed simultaneously with two opposing primers), and Fluorescent PCR-SSCP (in which PCR products are internally labelled with multiple fluorescent dyes, may be digested with restriction enzymes, followed by SSCP, and analysed on an automated DNA sequencer able to detect the fluorescent dyes).

Other methods which utilise the varying mobility of different nucleic acid structures include Denaturing Gradient Gel Electrophoresis (DGGE), Temperature Gradient Gel Electrophoresis (TGGE), and Heteroduplex Analysis (HET). Here, variation in the dissociation of double stranded DNA (for example, due to base-pair mismatches) results in a change in electrophoretic mobility. These mobility shifts are used to detect nucleotide variations.

Denaturing High Pressure Liquid Chromatography (HPLC) is yet a further method utilised to detect SNPs, using HPLC methods well-known in the art as an alternative to the separation methods described above (such as gel electophoresis) to detect, for example, homoduplexes and heteroduplexes which elute from the HPLC column at different rates, thereby enabling detection of mismatch nucleotides and thus SNPs.

Yet further methods to detect SNPs rely on the differing susceptibility of single stranded and double stranded nucleic acids to cleavage by various agents, including chemical cleavage agents and nucleolytic enzymes. For example, cleavage of mismatches within RNA:DNA heteroduplexes by RNase A, of heteroduplexes by, for example bacteriophage T4 endonuclease YII or T7 endonuclease I, of the 5′ end of the hairpin loops at the junction between single stranded and double stranded DNA by cleavase I, and the modification of mispaired nucleotides within heteroduplexes by chemical agents commonly used in Maxam-Gilbert sequencing chemistry, are all well known in the art.

Further examples include the Protein Translation Test (PTT), used to resolve stop codons generated by variations which lead to a premature termination of translation and to protein products of reduced size, and the use of mismatch binding proteins. Variations are detected by binding of, for example, the MutS protein, a component of Escherichia coli DNA mismatch repair system, or the human hMSH2 and GTBP proteins, to double stranded DNA heteroduplexes containing mismatched bases. DNA duplexes are then incubated with the mismatch binding protein, and variations are detected by mobility shift assay. For example, a simple assay is based on the fact that the binding of the mismatch binding protein to the heteroduplex protects the heteroduplex from exonuclease degradation.

Those skilled in the art will know that a particular SNP, particularly when it occurs in a regulatory region of a gene such as a promoter, can be associated with altered expression of a gene. Altered expression of a gene can also result when the SNP is located in the coding region of a protein-encoding gene, for example where the SNP is associated with codons of varying usage and thus with tRNAs of differing abundance. Such altered expression can be determined by methods well known in the art, and can thereby be employed to detect such SNPs. Similarly, where a SNP occurs in the coding region of a gene and results in a non-synonymous amino acid substitution, such substitution can result in a change in the function of the gene product. Similarly, in cases where the gene product is an RNA, such SNPs can result in a change of function in the RNA gene product. Any such change in function, for example as assessed in an activity or functionality assay, can be employed to detect such SNPs.

The above methods of detecting and identifying SNPs are amenable to use in the methods of the invention.

Of course, in order to detect and identify SNPs in accordance with the invention, a sample containing material to be tested is obtained from the subject. The sample can be any sample potentially containing the target SNPs (or target polypeptides, as the case may be) and obtained from any bodily fluid (blood, urine, saliva, etc) biopsies or other tissue preparations.

DNA or RNA can be isolated from the sample according to any of a number of methods well known in the art. For example, methods of purification of nucleic acids are described in Tijssen; Laboratory Techniques in Biochemistry and Molecular Biology: Hybridization with nucleic acid probes Part 1: Theory and Nucleic acid preparation, Elsevier, New York, N.Y. 1993, as well as in Maniatis, T., Fritsch, E. F. and Sambrook, J., Molecular Cloning Manual 1989.

To assist with detecting the presence or absence of polymorphisms/SNPs, nucleic acid probes and/or primers can be provided. Such probes have nucleic acid sequences specific for chromosomal changes evidencing the presence or absence of the polymorphism and are preferably labeled with a substance that emits a detectable signal when combined with the target polymorphism.

The nucleic acid probes can be genomic DNA or cDNA or mRNA, or any RNA-like or DNA-like material, such as peptide nucleic acids, branched DNAs, and the like. The probes can be sense or antisense polynucleotide probes. Where target polynucleotides are double-stranded, the probes may be either sense or antisense strands. Where the target polynucleotides are single-stranded, the probes are complementary single strands.

The probes can be prepared by a variety of synthetic or enzymatic schemes, which are well known in the art. The probes can be synthesized, in whole or in part, using chemical methods well known in the art (Caruthers et al., Nucleic Acids Res., Symp. Ser., 215-233 (1980)). Alternatively, the probes can be generated, in whole or in part, enzymatically.

Nucleotide analogs can be incorporated into probes by methods well known in the art. The only requirement is that the incorporated nucleotide analog must serve to base pair with target polynucleotide sequences. For example, certain guanine nucleotides can be substituted with hypoxanthine, which base pairs with cytosine residues. However, these base pairs are less stable than those between guanine and cytosine. Alternatively, adenine nucleotides can be substituted with 2,6-diaminopurine, which can form stronger base pairs than those between adenine and thymidine.

Additionally, the probes can include nucleotides that have been derivatized chemically or enzymatically. Typical chemical modifications include derivatization with acyl, alkyl, aryl or amino groups.

The probes can be immobilized on a substrate. Preferred substrates are any suitable rigid or semi-rigid support including membranes, filters, chips, slides, wafers, fibers, magnetic or nonmagnetic beads, gels, tubing, plates, polymers, microparticles and capillaries. The substrate can have a variety of surface forms, such as wells, trenches, pins, channels and pores, to which the polynucleotide probes are bound. Preferably, the substrates are optically transparent.

Furthermore, the probes do not have to be directly bound to the substrate, but rather can be bound to the substrate through a linker group. The linker groups are typically about 6 to 50 atoms long to provide exposure to the attached probe. Preferred linker groups include ethylene glycol oligomers, diamines, diacids and the like. Reactive groups on the substrate surface react with one of the terminal portions of the linker to bind the linker to the substrate. The other terminal portion of the linker is then functionalized for binding the probe.

The probes can be attached to a substrate by dispensing reagents for probe synthesis on the substrate surface or by dispensing preformed DNA fragments or clones on the substrate surface. Typical dispensers include a micropipette delivering solution to the substrate with a robotic system to control the position of the micropipette with respect to the substrate. There can be a multiplicity of dispensers so that reagents can be delivered to the reaction regions simultaneously.

Nucleic acid microarrays are preferred. Such microarrays (including nucleic acid chips) are well known in the art (see, for example U.S. Pat. Nos. 5,578,832; 5,861,242; 6,183,698; 6,287,850; 6,291,183; 6,297,018; 6,306,643; and 6,308,170, each incorporated by reference).

Alternatively, antibody microarrays can be produced. The production of such microarrays is essentially as described in Schweitzer & Kingsmore, “Measuring proteins on microarrays”, Curr Opin Biotechnol 2002; 13(1): 14-9; Avseekno et al., “Immobilization of proteins in immunochemical microarrays fabricated by electrospray deposition”, Anal Chem 2001 15; 73(24): 6047-52; Huang, “Detection of multiple proteins in an antibody-based protein microarray system, Immunol Methods 2001 1; 255 (1-2): 1-13.

The present invention also contemplates the preparation of kits for use in accordance with the present invention. Suitable kits include various reagents for use in accordance with the present invention in suitable containers and packaging materials, including tubes, vials, and shrink-wrapped and blow-molded packages.

Materials suitable for inclusion in an exemplary kit in accordance with the present invention comprise one or more of the following: gene specific PCR primer pairs (oligonucleotides) that anneal to DNA or cDNA sequence domains that flank the genetic polymorphisms of interest, reagents capable of amplifying a specific sequence domain in either genomic DNA or cDNA without the requirement of performing PCR; reagents required to discriminate between the various possible alleles in the sequence domains amplified by PCR or non-PCR amplification (e.g., restriction endonucleases, oligonucleotide that anneal preferentially to one allele of the polymorphism, including those modified to contain enzymes or fluorescent chemical groups that amplify the signal from the oligonucleotide and make discrimination of alleles more robust); reagents required to physically separate products derived from the various alleles (e.g. agarose or polyacrylamide and a buffer to be used in electrophoresis, HPLC columns, SSCP gels, formamide gels or a matrix support for MALDI-TOF).

It will be appreciated that the methods of the invention can be performed in conjunction with an analysis of other risk factors known to be associated with ACS. Such risk factors include epidemiological risk factors associated with an increased risk of developing ACS. Such risk factors include, but are not limited to smoking and/or exposure to tobacco smoke, age, sex and familial history. These risk factors can be used to augment an analysis of one or more polymorphisms as herein described when assessing a subject's risk of developing ACS.

The predictive methods of the invention allow a number of therapeutic interventions and/or treatment regimens to be assessed for suitability and implemented for a given subject. The simplest of these can be the provision to the subject of motivation to implement a lifestyle change, for example, where the subject is a current smoker, the methods of the invention can provide motivation to quit smoking.

The manner of therapeutic intervention or treatment will be predicated by the nature of the polymorphism(s) and the biological effect of said polymorphism(s). For example, where a susceptibility polymorphism is associated with a change in the expression of a gene, intervention or treatment is preferably directed to the restoration of normal expression of said gene, by, for example, administration of an agent capable of modulating the expression of said gene. Where a polymorphism is associated with decreased expression of a gene, therapy can involve administration of an agent capable of increasing the expression of said gene, and conversely, where a polymorphism is associated with increased expression of a gene, therapy can involve administration of an agent capable of decreasing the expression of said gene. Methods useful for the modulation of gene expression are well known in the art. For example, in situations where a polymorphism is associated with upregulated expression of a gene, therapy utilising, for example, RNAi or antisense methodologies can be implemented to decrease the abundance of mRNA and so decrease the expression of said gene. Alternatively, therapy can involve methods directed to, for example, modulating the activity of the product of said gene, thereby compensating for the abnormal expression of said gene.

Where a susceptibility polymorphism is associated with decreased gene product function or decreased levels of expression of a gene product, therapeutic intervention or treatment can involve augmenting or replacing of said function, or supplementing the amount of gene product within the subject for example, by administration of said gene product or a functional analogue thereof. For example, where a polymorphism is associated with decreased enzyme function, therapy can involve administration of active enzyme or an enzyme analogue to the subject. Similarly, where a polymorphism is associated with increased gene product function, therapeutic intervention or treatment can involve reduction of said function, for example, by administration of an inhibitor of said gene product or an agent capable of decreasing the level of said gene product in the subject. For example, where a SNP allele or genotype is associated with increased enzyme function, therapy can involve administration of an enzyme inhibitor to the subject.

Likewise, when a protective polymorphism is associated with upregulation of a particular gene or expression of an enzyme or other protein, therapies can be directed to mimic such upregulation or expression in an individual lacking the resistive genotype, and/or delivery of such enzyme or other protein to such individual Further, when a protective polymorphism is associated with downregulation of a particular gene, or with diminished or eliminated expression of an enzyme or other protein, desirable therapies can be directed to mimicking such conditions in an individual that lacks the protective genotype.

The relationship between the various polymorphisms identified above and the susceptibility (or otherwise) of a subject to ACS also has application in the design and/or screening of candidate therapeutics. This is particularly the case where the association between a polymorphism predictive of susceptibility is manifested by either an upregulation or downregulation of expression of a gene. In such instances, the effect of a candidate therapeutic on such upregulation or downregulation is readily detectable.

For example, in one embodiment existing human vascular organ and cell cultures are screened for SNP genotypes as set forth above. (For information on human vascular organ and cell cultures, see for example: Clare Wise ED., Epithelial Cell Culture Protocols, 2002, ISBN 0896038939, Humana Press Inc. NJ; Endothelial Cell Culture, Roy Bicknell, ED., 1996, ISBN 0521550246, Cambridge University Press, UK; Cell Culture Models of Biological Barriers, Claus-Michael Lehr, ED., 2002, ISBN 0415277248, Taylor and Francis, UK; each of which is hereby incorporated by reference in its entirety.) Cultures representing relevant genotype groups are selected, together with cultures which are putatively “normal” in terms of the expression of a gene which is either upregulated or downregulated where a polymorphism is present.

Samples of such cultures are exposed to a library of candidate therapeutic compounds and screened for: (a) downregulation of genes that are normally upregulated in susceptible genotypes; or (b) upregulation of genes that are normally downregulated in susceptible genotypes. Compounds are selected for their ability to alter the regulation and/or action of genes in a culture having a susceptible genotype.

Similarly, where the polymorphism is one which when present results in a physiologically active concentration of an expressed gene product outside of the normal range for a subject (adjusted for age and sex), and where there is an available prophylactic or therapeutic approach to restoring levels of that expressed gene product to within the normal range, individual subjects can be screened to determine the likelihood of their benefiting from that restorative approach. Such screening involves detecting the presence or absence of the polymorphism in the subject by any of the methods described herein, with those subjects in which the polymorphism is present being identified as individuals likely to benefit from treatment.

The invention will now be described in more detail, with reference to the following non-limiting examples.

### Example 1

### Case Association Study

### Introduction

Case-control association studies allow the careful selection of a control group where matching for important risk factors is critical. In this study, smokers diagnosed with ACS and smokers without ACS were compared. This unique control group is highly relevant as it is impossible to pre-select smokers with zero risk of ACS—i.e., those who although smokers will never develop ACS. Smokers with a high pack year history and normal cardiovascular function were used as a “low risk” group of smokers, as the Applicants believe it is not possible with current knowledge to identify a lower risk group of smokers. The Applicants believe, without wishing to be bound by any theory, that this approach allows for a more rigorous comparison of low penetrant, high frequency polymorphisms that may confer an increased risk of developing ACS. The Applicants also believe, again without wishing to be bound by any theory, that there may be polymorphisms that confer a degree of protection from ACS which may only be evident if a smoking cohort with normal cardiovascular function is utilised as a comparator group. Thus, smokers with ACS would be expected to have a lower frequency of these polymorphisms compared to smokers with normal cardiovascular function and no diagnosed ACS.

Subjects of European decent who had smoked a minimum of fifteen pack years and diagnosed with acute coronary syndrome (ACS, including acute myocardial infarction and unstable angina) were recruited. Subjects met the following criteria: diagnosed with ACS based on clinical presentation (history, ECG, cardiac biomarker assays) to a tertiary care hospital. Subjects with ACS had had coronary angiograms that confirmed the presence of atheromatous disease of the coronary arteries. Subjects with ACS were aged between 40-60 yrs old and of European descent. One hundred and forty-eight subjects were recruited, of these 85% were male, the mean FEV1/FVC (±1 SD) was 74% (±8), mean FEV1 as a percentage of predicted was 94 (±15). Mean age, cigarettes per day and pack year history was 50 yrs (±3), 22 cigarettes/day (±8) and 31 pack years (±11), respectively. Four hundred and sixty European subjects who had smoked a minimum of fifteen pack years and who had never suffered from angina, chest pain, suffered a heart attack, or had been diagnosed with ischaemic heart disease in the past were also studied. This control group was recruited through community based volunteers who were ex-smokers or current smokers, and consisted 55% male, with a mean FEV1/FVC (±1 SD) of 75% (±9), and mean FEV1 as a percentage of predicted was 98 (±12). Mean age, cigarettes per day and pack year history was 60 yrs (±10), 23 cigarettes/day (±11) and 40 pack years (±21), respectively.

This study shows that polymorphisms found in greater frequency in acute coronary syndrome patients compared to resistant smokers may reflect an increased susceptibility to the development of life-threatening acute coronary syndrome. Similarly, polymorphisms found in greater frequency in resistant smokers compared to acute coronary syndrome patients may reflect a protective role.

### Summary of Characteristics for the Acs Cohort and Resistant Control Smokers.

| | Acute Coronary | | Resistant | | |
|---|---|---|---|---|---|
| Parameter | syndrome | | smokers | | |
| Mean (1SD) | N = 148 | | N = 460 | | Differences |
| % male | 85% | | 55% | | P < 0.05 |
| Age (yrs) | 50 | (3) | 60 | (10) | P < 0.05 |
| Pack years | 31 | (11) | 40 | (21) | P < 0.05 |
| Cigarettes/day | 22 | (8) | 23 | (11) | ns |
| FEV1 (L) | 3.3 | (0.7) | 2.7 | (0.6) | P < 0.05 |
| FEV1 % predict | 94 | (15) | 98% | (12) | P < 0.05 |
| FEV1/FVC | 74 | (8) | 75 | (9) | P < 0.05 |

Means and 1SD

### Genotyping Methods

### Polymorphism Genotyping Using the Sequenom Autoflex Mass Spectrometer

Genomic DNA was extracted from whole blood samples (Maniatis, T., Fritsch, E. F. and Sambrook, J., Molecular Cloning Manual. 1989). Purified genomic DNA was aliquoted (10 ng/ul concentration) into 96 well plates and genotyped on a Sequenom™ system (Sequenom™ Autoflex Mass Spectrometer and Samsung 24 pin nanodispenser) using the following sequences, amplification conditions and methods.

The following conditions were used for the PCR multiplex reaction: final concentrations were for 10×Buffer 15 mM MgCl₂ 1.25×, 25 mM MgCl₂ 1.625 mM, dNTP mix 25 mM 500 uM, primers 4 uM 100 nM, Taq polymerase (Quiagen hot start) 0.15 U/reaction, Genomic DNA 10 ng/ul. Cycling times were 95° C. for 15 min, (5° C. for 15 s, 56° C. 30 s, 72° C. 30 s for 45 cycles with a prolonged extension time of 3 min to finish. We used shrimp alkaline phosphatase (SAP) treatment (2 ul to 5 ul per PCR reaction) incubated at 35° C. for 30 min and extension reaction (add 2 ul to 7 ul after SAP treatment) with the following volumes per reaction of: water, 0.76 ul; hME 10× termination buffer, 0.2 ul; hME primer (10 uM), 1 ul; Mass EXTEND enzyme, 0.04 ul. See Tables 1-26 for full name of SNPs and candidate genes.

Sequenom conditions for PCR and Mass spectrometer genotyping

| | | | |
|---|---|---|---|
| SNP_ID | SNP Name | 2nd-PCRP | 1st-PCRP |

| | | | |
|---|---|---|---|
| PDGFA | PDGFA 12 IN5 C/T | ACGTTGGATGAAGGCTCTGAAGACCTGTTC | ACGTTGGATGATCCGGATTATCGGGAAGAG |
| | | [SEQ.ID.NO. 1] | [SEQ.ID.NO. 2] |
| RS17580 | 1-antitrypsin S allele | ACGTTGGATGCTTGGTGATGATATCGTGGG | ACGTTGGATGTCTTCTTCCTGCCTGATGAG |
| | | [SEQ.ID.NO. 3] | [SEQ.ID.NO. 4] |
| RS1799983 | NOS3 298 G/T | ACGTTGGATGAAACGGTCGCTTCGACGTG | ACGTTGGATGGGGCAGAAGGAAGAGTTC |
| | | [SEQ.ID.NO. 5] | [SEQ.ID.NO. 6] |
| RS1800469 | TGFB1 −509 C/T | ACGTTGGATGTACAGGTGTCTGCCTCCTGA | ACGTTGGATGAAGAGGGTCTGTCAACATGG |
| | | [SEQ.ID.NO. 7] | [SEQ.ID.NO. 8] |
| RS1151640 | OG13G1 132 A/G | ACGTTGGATGATAGCCATGACCATGCTGAG | ACGTTGGATGGGCCATTTGTTTCCCTCTTC |
| | | [SEQ.ID.NO. 9] | [SEQ.ID.NO. 10] |
| RS2276109 | MMP12 −82 A/G | ACGTTGGATGTTGAGATAGATCAAGGGATG | ACGTTGGATGGTCCGGGTTCTGTGAATATG |
| | | [SEQ.ID.NO. 11] | [SEQ.ID.NO. 12] |
| RS4986790 | TLR4 299 A/G | ACGTTGGATGAGCATACTTAGACTACTACC | ACGTTGGATGCACACTCACCAGGGAAAATG |
| | | [SEQ.ID.NO. 13] | [SEQ.ID.NO. 14] |
| RS1800896 | IL-10 −1084 A/G | ACGTTGGATGATTCCATGGAGGCTGGATAG | ACGTTGGATGGACAACACTACTAAGGCTTC |
| | | [SEQ.ID.NO. 15] | [SEQ.ID.NO. 16] |
| RS5498 | ICAM1 K469E A/G | ACGTTGGATGACTCACAGAGCACATTCACG | ACGTTGGATGTGTCACTCGAGATCTTGAGG |
| | | [SEQ.ID.NO. 17] | [SEQ.ID.NO. 18] |
| RS1449683 | FGF2 Ser52Ser C/T | ACGTTGGATGAGGCGGCGTCCGCGGAGACA | ACGTTGGATGCTCGGCCGCTCTTCTGTCC |
| | | [SEQ.ID.NO. 19] | [SEQ.ID.NO. 20] |
| RS2239527 | BAT1 −23 C/G | ACGTTGGATGTTACCTAAACAGGGAGAGCG | ACGTTGGATGAAGCCTGCAACCGGAAGTG |
| | | [SEQ.ID.NO. 21] | [SEQ.ID.NO. 22] |
| RS1799895 | SOD3 Arg213Gly C/G | ACGTTGGATGCTCAGGCGGCCTTGCACTC | ACGTTGGATGAGGCGCGGGAGCACTCAGA |
| | | [SEQ.ID.NO. 23] | [SEQ.ID.NO. 24] |
| RS1800875 | CMA1 −1903 A/G | ACGTTGGATGGCTCCACAGCATCAAGATTC | ACGTTGGATGTTCCATTTCCTCACCCTCAG |
| | | [SEQ.ID.NO. 25] | [SEQ.ID.NO. 26] |
| RS1719134 | MIP1A +459 C/T | ACGTTGGATGGGTTCAAGAAGTCATACCCC | ACGTTGGATGAGCTCTGTCCCTTGGATGTC |
| | | [SEQ.ID.NO. 27] | [SEQ.ID.NO. 28] |
| RS2243250 | IL-4 −589 C/T | ACGTTGGATGCGACCTGTCCTTCTCAAAAC | ACGTTGGATGGAATAACAGGCAGACTCTCC |
| | | [SEQ.ID.NO. 29] | [SEQ.ID.NO. 30] |
| RS1801275 | IL-4RA Q576R A/G | ACGTTGGATGGAAATGTCCTCCAGCATGGG | ACGTTGGATGACCCTGCTCCACCGCATGTA |
| | | [SEQ.ID.NO. 31] | [SEQ.ID.NO. 32] |
| RS2227956 | HASP70 Horn T2437C | ACGTTGGATGTGATCTTGTTCACCTTGCCG | ACGTTGGATGCGAGGTGACGTTTGACATTG |
| | | [SEQ.ID.NO. 33] | [SEQ.ID.NO. 34] |
| RS1799750 | MMP1 −1607 1G/2G | ACGTTGGATGCTTCAGTATATCTTGGATTG | ACGTTGGATGGTTATGCCACTTAGATGAGG |
| | | [SEQ.ID.NO. 35] | [SEQ.ID.NO. 36] |
| RS17880821 | MMP7 −181 A/G | ACGTTGGATGGGAGTCAATTTATGCAGCAG | ACGTTGGATGCATCGTTATTGGCAGGAAGC |
| | | [SEQ.ID.NO. 37] | [SEQ.ID.NO. 38] |
| RS4986791 | TLR4 399 C/T | ACGTTGGATGAGCCCAAGAAGTTTGAACTC | ACGTTGGATGAGGTTGCTGTTCTCAAAGTG |
| | | [SEQ.ID.NO. 39] | [SEQ.ID.NO. 40] |
| RS1041981 | LTA Thr26Asn A/C | ACGTTGGATGGAGGTCAGGTGGATGTTTAC | ACGTTGGATGACCCCAAGATGCATCTTGCC |
| | | [SEQ.ID.NO. 41] | [SEQ.ID.NO. 42] |
| PDGFRA | PDGFRA −1630 I/D | ACGTTGGATGGGCAACTAGCCTAAAAACCC | ACGTTGGATGCAGAGTGCGGAATAAAAGGC |
| | | [SEQ.ID.NO. 43] | [SEQ.ID.NO. 44] |
| GCLM | GCLM −588 C/T | ACGTTGGATGTGAGGTAGACACCGCCTCC | ACGTTGGATGAAGAGACGTGTAGGAAGCCC |
| | | [SEQ.ID.NO. 45] | [SEQ.ID.NO. 46] |
| RS2430561 | IGN-G 874 A/T | ACGTTGGATGCAGACATTCACAATTGATT | ACGTTGGATGGATAGTTCCAAACATGTGCG |
| | | [SEQ.ID.NO. 47] | [SEQ.ID.NO. 48] |
| RS2071592 | NFKBIL1 −63 T/A | ACGTTGGATGTAACGCCCCTCACAGTTCAC | ACGTTGGATGACTCCAGGCTGGAGGAAATG |
| | | [SEQ.ID.NO. 49] | [SEQ.ID.NO. 50] |
| PAI1 | PAI-1 −668 4G/5G | ACGTTGGATGCACAGAGAGAGTCTGGACAC | ACGTTGGATGTCTTGGTCTTTCCCTCATCC |
| | | [SEQ.ID.NO. 51] | [SEQ.ID.NO. 52] |
| rs2066845 | Caspase | ACGTTGGATGGTCTGTTGACTCTTTTGGC | ACGTTGGATGTGGTGATCACCCAAGGCTTC |
| | | [SEQ.ID.NO. 105] | [SEQ.ID.NO. 106] |
| rs3732379 | CX3CR1 | ACGTTGGATGCATAGAGCTTAAGCGTCTCC | ACGTTGGATGTGATCCTTCTGGTGGTCATC |
| | | [SEQ.ID.NO. 107] | [SEQ.ID.NO. 108] |
| Cathespin G | Cathepsin G Asn125Ser | ACGTTGGATGTCAGTCCCTCCTGGGCTCTA | ACGTTGGATGAGAAGAGTCAGACGGAATCG |
| | | [SEQ.ID.NO. 109] | [SEQ.ID.NO. 110] |
| rs6520277 | TIMP1 | ACGTTGGATGGACTCTTGCACATCACTACC | ACGTTGGATGAGTGTAGGTCTTGGTGAAGC |
| | | [SEQ.ID.NO. 111] | [SEQ.ID.NO. 112] |

Sequenom conditions for PCR and Mass spectrometer genotyping

| SNP_ID | SNP Name | AMP_LEN | UP_CONF | MP_CONF | Tm(NN) | PcGC | PWARN | UEP_DIR | UEP_MASS |
|---|---|---|---|---|---|---|---|---|---|
| PDGFA | PDGFA 12 IN5 C/T | 100 | 100 | 65.1 | 50.4 | 62.5 | | R | 5204.4 |
| RS17580 | 1-antitrypsin S allele | 91 | 99.7 | 65.1 | 47.3 | 47.1 | | R | 5905.8 |
| RS1799983 | NOS3 298 G/T | 93 | 87.8 | 65.1 | 59.9 | 63.2 | sDh | F | 6143 |
| RS1800469 | TGFB1 −509 C/T | 120 | 94.5 | 65.1 | 57.5 | 65 | | F | 6543.2 |
| RS1151640 | OG13G1 132 A/G | 101 | 100 | 65.1 | 48.4 | 38.1 | d | R | 6718.4 |
| RS2276109 | MMP12 −82 A/G | 88 | 93.8 | 65.1 | 48.9 | 34.8 | D | F | 7095.6 |
| RS4986790 | TLR4 299 A/G | 105 | 94.3 | 65.1 | 49.6 | 41.7 | D | F | 7271.8 |
| RS1800896 | IL-10 −1084 A/G | 107 | 98.4 | 65.1 | 57.6 | 50 | D | R | 8272.4 |
| RS5498 | ICAM1 K469E A/G | 115 | 99.1 | 70.7 | 47.9 | 50 | d | R | 4801.1 |
| RS1449683 | FGF2 Ser52Ser C/T | 115 | 55.7 | 70.7 | 52.6 | 68.8 | d | F | 4836.2 |
| RS2239527 | BAT1 −23 C/G | 102 | 95.9 | 70.7 | 48.9 | 52.9 | | R | 5317.5 |
| RS1799895 | SOD3 Arg213Gly C/G | 83 | 66.2 | 70.7 | 61.6 | 82.4 | sD | R | 5652.7 |
| RS1800875 | CMA1 −1903 A/G | 98 | 100 | 70.7 | 45.9 | 36.8 | d | R | 5777.8 |
| RS1719134 | MIP1A +459 C/T | 92 | 98.3 | 70.7 | 50.9 | 52.9 | D | R | 5784.8 |
| RS2243250 | IL-4 −589 C/T | 100 | 100 | 70.7 | 47.6 | 36.8 | dH | F | 6205.1 |
| RS1801275 | IL-4RA Q576R A/G | 109 | 90.4 | 70.7 | 55 | 66.7 | d | F | 6886.5 |
| RS2227956 | HASP70 Hom T2437C | 103 | 100 | 70.7 | 56.4 | 63.2 | D | R | 6966.5 |
| RS1799750 | MMP1 −1607 1G/2G | 104 | 86.4 | 70.7 | 45.4 | 29.2 | H | R | 7405.8 |
| RS17880821 | MMP7 −181 A/G | 99 | 98.6 | 70.7 | 48.4 | 28 | d | F | 7684.1 |
| RS4986791 | TLR4 399 C/T | 118 | 95.9 | 70.7 | 46.2 | 36.4 | D | R | 8025.2 |
| RS1041981 | LTA Thr26Asn A/C | 94 | 98.4 | 71.3 | 50.6 | 58.8 | d | R | 5315.5 |
| PDGFRA | PDGFRA −1630 I/D | 100 | 100 | 71.3 | 48.2 | 42.1 | | F | 5740.8 |
| GCLM | GCLM −588 C/T | 116 | 88.4 | 71.3 | 61.5 | 71.4 | D | R | 6350.1 |
| RS2430561 | IGN-G 874 A/T | 112 | 75.9 | 71.3 | 46.4 | 26.1 | | F | 6943.6 |
| RS2071592 | NFKBIL1 −63 T/A | 91 | 96.8 | 94.1 | 50.8 | 62.5 | d | R | 4713.1 |
| PAI1 | PAI-1 −668 4G/5G | 108 | 98.3 | 94.1 | 54.1 | 64.7 | g | F | 5291.4 |
| rs2066845 | Caspase | 113 | 95.8 | 61.5 | 50.5 | 47.4 | D | | |
| rs3732379 | CX3CR1 | 104 | 99.9 | 61.5 | 52.8 | 39.1 | | | |
| Cathespin G | Cathepsin G Asn125Ser | 84 | 88.6 | 61.5 | 56.4 | 63.2 | D | | |
| rs6520277 | TIMP1 | 109 | 99.7 | 61.5 | 47.2 | 44.4 | d | | |

Sequenom conditions for PCR and Mass spectrometer genotyping

| | | | | | |
|---|---|---|---|---|---|
| | | | EXT1_{—} | EXT1_{—} | |
| SNP_ID | SNP Name | UEP_SEQ | CALL | MASS | EXT1_SEQ |

| | | | | | |
|---|---|---|---|---|---|
| PDGFA | PDGFA 12 IN5 | tCACGATGCCGACGAAG | T | 5475.6 | tCACGATGCCGACGAAGA |
| | C/T | [SEQ.ID.NO. 53] | | | [SEQ.ID.NO. 54] |
| RS17580 | 1-antitrypsin S | ggCGTGGGTGAGTTCATTT | T | 6177 | ggCGTGGGTGAGTTCATTTA |
| | allele | [SEQ.ID.NO. 55] | | | [SEQ.ID.NO. 56] |
| RS1799983 | NOS3 298 G/T | gTGCTGCAGGCCCCAGATGA | G | 6430.2 | gTGCTGCAGGCCCCAGATGAG |
| | | [SEQ.ID.NO. 57] | | | [SEQ.ID.NO. 58] |
| RS1800469 | TGFB1 −509 | cgGCCTCCTGACCCTTCCATCC | C | 6790.4 | cgGCCTCCTGACCCTTCCATCCC |
| | C/T | [SEQ.ID.NO. 59] | | | [SEQ.ID.NO. 60] |
| RS1151640 | OG13G1 132 | cCACACATATGGTGGTTCATAA | G | 6965.6 | cCACACATATGGTGGTTCATAAC |
| | A/G | [SEQ.ID.NO. 61] | | | [SEQ.ID.NO. 62] |
| RS2276109 | MMP12 −82 | TAGATCAAGGGATGATATCAACT | A | 7366.9 | TAGATCAAGGGATGATATCAACTA |
| | A/G | [SEQ.ID.NO. 63] | | | [SEQ.ID.NO. 64] |
| RS4986790 | TLR4 299 A/G | CATACTTAGACTACTACCTCGATG | A | 7543 | CATACTTAGACTACTACCTCGATGA |
| | | [SEQ.ID.NO. 65] | | | [SEQ.ID.NO. 66] |
| RS1800896 | IL-10 −1084 | CTTTCCTCTTACCTATCCCTACTTCCCC | G | 8519.6 | CTTTCCTCTTACCTATCCCTACTTCCCCC |
| | A/G | [SEQ.ID.NO. 67] | | | [SEQ.ID.NO. 68] |
| RS5498 | ICAM1 K469E | ACATTCACGGTCACCT | G | 5048.3 | ACATTCACGGTCACCTC |
| | A/G | [SEQ.ID.NO. 69] | | | [SEQ.ID.NO. 70] |
| RS1449683 | FGF2 | CGCGGAGACACCCATC | C | 5083.3 | CGCGGAGACACCCATCC |
| | Ser52Ser C/T | [SEQ.ID.NO. 71] | | | [SEQ.ID.NO. 72] |
| RS2239527 | BAT1 −23 C/G | CGACGAAGGAGGGAAAT | G | 5564.7 | CGACGAAGGAGGGAAATC |
| | | [SEQ.ID.NO. 73] | | | [SEQ.ID.NO. 74] |
| RS1799895 | SOD3 | tcCTCGCTCTCGCGCCGCC | G | 5899.8 | tcCTCGCTCTCGCGCCGCCC |
| | Arg213Gly C/G | [SEQ.ID.NO. 75] | | | [SEQ.ID.NO. 76] |
| RS1800875 | CMA1 −1903 | CCAAGACTTAAGTTTTGCT | G | 6025 | CCAAGACTTAAGTTTTGCTC |
| | A/G | [SEQ.ID.NO. 77] | | | [SEQ.ID.NO. 78] |
| RS1719134 | MIP1A +459 | ggACCCCAACCCAAGAGAA | T | 6056 | ggACCCCAACCCAAGAGAAA |
| | C/T | [SEQ.ID.NO. 79] | | | [SEQ.ID.NO. 80] |
| RS2243250 | IL-4 −589 C/T | gAAACTTGGGAGAACATTGT | C | 6452.2 | gAAACTTGGGAGAACATTGTC |
| | | [SEQ.ID.NO. 81] | | | [SEQ.ID.NO. 82] |
| RS1801275 | IL-4RA Q576R | ttcttCCCCCACCAGTGGCTATC | A | 7157.7 | ttcttCCCCCACCAGTGGCTATCA |
| | A/G | [SEQ.ID.NO. 83] | | | [SEQ.ID.NO. 84] |
| RS2227956 | HASP70 Horn | acaaCTTGCCGGTGCTCTTGTCC | T | 7237.7 | acaaCTTGCCGGTGCTCTTGTCCA |
| | T2437C | [SEQ.ID.NO. 85] | | | [SEQ.ID.NO. 86] |
| RS1799750 | MMP1 −1607 | GATTGATTTGAGATAAGTCATATC | G | 7653 | GATTGATTTGAGATAAGTCATATCC |
| | 1G/2G | [SEQ.ID.NO. 87] | | | [SEQ.ID.NO. 88] |
| RS17880821 | MMP7 −181 | CAGAAAAAAAAATCCTTTGAAAGAC | A | 7955.3 | CAGAAAAAAAAATCCTTTGAAAGACA |
| | A/G | [SEQ.ID.NO. 89] | | | [SEQ.ID.NO. 90] |
| RS4986791 | TLR4 399 C/T | ggtgCAGATCTAAATACTTTAGGCTG | T | 8296.4 | ggtgCAGATCTAAATACTTTAGGCTGA |
| | | [SEQ.ID.NO. 91] | | | [SEQ.ID.NO. 92] |
| RS1041981 | LTA Thr26Asn | GAGCAGCAGGTTTGAGG | C | 5602.7 | GAGCAGCAGGTTTGAGGG |
| | A/C | [SEQ.ID.NO. 93] | | | [SEQ.ID.NO. 94] |
| PDGFRA | PDGFRA −1630 | TAAAAACCCGGTTCTCAAC | DEL | 6012 | TAAAAACCCGGTTCTCAACA |
| | I/D | [SEQ.ID.NO. 95] | | | [SEQ.ID.NO. 96] |
| GCLM | GCLM −588 | CTCCGCCTGGTGAGGTCTCCC | T | 6621.3 | CTCCGCCTGGTGAGGTCTCCCA |
| | C/T | [SEQ.ID.NO. 97] | | | [SEQ.ID.NO. 98] |
| RS2430561 | IGN-G 874 A/T | TTCTTACAACACAAAATCAAATC | A | 7214.8 | TTCTTACAACACAAAATCAAATCA |
| | | [SEQ.ID.NO. 99] | | | [SEQ.ID.NO. 100] |
| RS2071592 | NFKBIL1 −63 | ACTTCCGTCCTCCACC | T | 4984.3 | ACTTCCGTCCTCCACCA |
| | T/A | [SEQ.ID.NO. 101] | | | [SEQ.ID.NO. 102] |
| PAI1 | PAI-1 −668 | AGTCTGGACACGTGGGG | DEL | 5562.6 | AGTCTGGACACGTGGGGA |
| | 4G/5G | [SEQ.ID.NO. 103] | | | [SEQ.ID.NO. 104] |

Sequenom conditions for PCR and Mass spectrometer genotyping

| | | | | | | | |
|---|---|---|---|---|---|---|---|
| | | UEP_{—} | UEP_{—} | | EXT1_{—} | EXT1_{—} | |
| SNP_ID | SNP | DIR | MASS | UEP_SEQ | CALL | MASS | EXT1_SEQ |

| | | | | | | | |
|---|---|---|---|---|---|---|---|
| rs2066845 | Caspase | F | 5800.8 | TGGCCTTTTCAGATTCTGG | C | 6048 | TGGCCTTTTCAGATTCTGGC |
| | | | | [SEQ.ID.NO. 113] | | | [SEQ.ID.NO. 114] |
| rs3732379 | CX3CR1 | F | 7049.6 | AAGCGTCTCCAGGAAAATCATAA | C | 7296.8 | AAGCGTCTCCAGGAAAATCATAAC |
| | | | | [SEQ.ID.NO. 115] | | | [SEQ.ID.NO. 116] |
| Cathespin G | Cathepsin G | R | 7458.8 | tgggaTCTAGGCAGAGCCACTGGG | G | 7706 | tgggaTCTAGGCAGAGCCACTGGGC |
| | Asn125Ser | | | [SEQ.ID.NO. 117] | | | [SEQ.ID.NO. 118] |
| rs6520277 | TIMP1 | F | 5987.9 | ccCATCACTACCTGCAGTTT | C | 6235.1 | ccCATCACTACCTGCAGTTTC |
| | | | | [SEQ.ID.NO. 119] | | | [SEQ.ID.NO. 120] |

Sequenom conditions for PCR and Mass spectrometer genotyping

| | | | | |
|---|---|---|---|---|
| SNP_ID | SNP | EXT2_CALL | EXT2_MASS | EXT2_SEQ |

| | | | | |
|---|---|---|---|---|
| rs2066845 | Caspase | G | 6088 | TGGCCTTTTCAGATTCTGGG |
| | | | | [SEQ.ID.NO. 121] |
| rs3732379 | CX3CR1 | T | 7376.7 | AAGCGTCTCCAGGAAAATCATAAT |
| | | | | [SEQ.ID.NO. 122] |
| Asn125Ser | Cathepsin G | A | 7785.9 | tgggaTCTAGGCAGAGCCACTGGGT |
| | | | | [SEQ.ID.NO. 123] |
| rs6520277 | TIMP1 | T | 6315 | ccCATCACTACCTGCAGTTTT |
| | | | | [SEQ.ID.NO. 124] |

### Results

TABLE 1
Chymase 1 (CMA) −1903 A/G polymorphism allele and genotype
frequencies in the ACS patients and resistant smokers.

| | Allele* | | Genotype | | |
|---|---|---|---|---|---|
| Frequency | A | G | AA | AG | GG |
| ACS | 135 (48%) | 149 (52%) | 38 (27%) | 59 (42%) | 45 (32%) |
| n = 142 (%) | | | | | |
| Resistant | 508 (56%) | 396 (44%) | 145 (32%) | 218 (48%) | 89 (20%) |
| n = 452 (%) | | | | | |

*number of chromosomes (2n)

Genotype. GG vs AG/AA for ACS vs resistant smoker controls, Odds ratio (OR)=1.9, 95% confidence limits 1.2-3.0, χ² (Mantel-Haenszel)=8.23, p=0.004,

GG genotype=susceptibility

Allele G vs A, ACS vs resistant smoker controls, Odds ratio (OR)=1.4, 95% confidence limits 1.1-1.9, χ² (Mantel-Haenszel)=6.52, p=0.01,

G allele=susceptibility

TABLE 2
Transforming growth factor beta 1 (TGFB1) −509
C/T polymorphism allele and genotype frequencies
in the ACS patients and resistant smokers.

| | Allele* | | Genotype | | |
|---|---|---|---|---|---|
| Frequency | C | T | CC | CT | TT |
| ACS | 222 (76%) | 72 (24%) | 84 (57%) | 54 (37%) | 9 (6%) |
| n = 147 (%) | | | | | |
| Resistant | 636 (70%) | 278 (30%) | 216 (47%) | 204 (45%) | 37 (8%) |
| n = 457 (%) | | | | | |

*number of chromosomes (2n)

Genotype. CC vs CT/TT for ACS vs resistant smoker controls, Odds ratio (OR)=1.5, 95% confidence limits 1.0-2.2, χ² (Mantel-Haenszel) 3.96, p=0.05,

CC genotype=susceptibility

Allele. C vs T for ACS vs resistant smoker controls, Odds ratio (OR)=1.4, 95% confidence limits 1.0-1.8, χ² (Mantel-Haenszel)=3.79, p=0.05,

C allele=susceptibility

TABLE 3
Matrix metalloproteinase 12 (MMP12) −82 A/G
polymorphism allele and genotype frequencies in
the ACS patients and resistant smokers.

| | Allele* | | Genotype | | |
|---|---|---|---|---|---|
| Frequency | A | G | AA | AG | GG |
| ACS | 249 (86%) | 39 (14%) | 110 (76%) | 29 (20%) | 5 (4%) |
| n = 144 (%) | | | | | |
| Resistant | 799 (88%) | 113 (12%) | 348 (76%) | 103 (23%) | 5 (1%) |
| n = 456 (%) | | | | | |

*number of chromosomes (2n)

Genotype. GG vs AA/AG for ACS vs resistant smoker controls, Odds ratio (OR)=3.2, 95% confidence limits 0.8-13, χ² (Mantel-Haenszel)=3.76, p=0.05,

GG genotype=susceptibility

TABLE 4
Fibroblast growth factor 2 (FGF2) Ser 52 Ser (223
C/T) polymorphism allele and genotype frequencies
in the ACS patients and resistant smokers.

| | Allele* | | Genotype | | |
|---|---|---|---|---|---|
| Frequency | C | T | CC | CT | TT |
| ACS | 252 (88%) | 34 (12%) | 112 (78%) | 28 (20%) | 3 (2%) |
| n = 143 (%) | | | | | |
| Resistant | 823 (92%) | 75 (8%) | 380 (85%) | 63 (14%) | 6 (1%) |
| n = 449 (%) | | | | | |

*number of chromosomes (2n)

Genotype. CT/TT vs CC for ACS vs resistant smoker controls, Odds ratio (OR)=1.5, 95% confidence limits 0.9-2.5, χ² (Mantel-Haenszel)=3.1, p=0.08,

CT/TT genotype=susceptibility (CC protective)

Allele. T vs C for ACS vs resistant smoker controls, Odds ratio (OR)=1.5, 95% confidence limits 0.9-2.3, χ² (Mantel-Haenszel)=3.24, p=0.07,

T allele=susceptibility

TABLE 5
Interleukin 4 receptor alpha (IL4RA) Q576R A/G
polymorphism allele and genotype frequencies
in the ACS patients and resistant smokers.

| | Allele* | | Genotype | | |
|---|---|---|---|---|---|
| Frequency | A | G | AA | AG | GG |
| ACS | 223 (75%) | 73 (25%) | 85 (57%) | 53 (36%) | 10 (7%) |
| n = 148 (%) | | | | | |
| Resistant | 751 (82%) | 169 (18%) | 303 (66%) | 145 (32%) | 12 (3%) |
| n = 460 (%) | | | | | |

*number of chromosomes (2n)

Genotype. GG vs AA/AG for ACS vs resistant smoker controls, Odds ratio (OR)=2.71, 95% confidence limits 1.1-6.9, χ² (Mantel-Haenszel)=5.52, p=0.02,

GG genotype=susceptibility

Genotype. AA vs AG/GG for ACS vs resistant smoker controls, Odds ratio (OR)=0.47, 95% confidence limits 0.07-1.0, χ² (Mantel-Haenszel)=3.45, p=0.05,

AA genotype=protective

Allele. G vs A for ACS vs resistant smoker controls, Odds ratio (OR)=1.5, 95% confidence limits 1.1-2.0, χ² (Mantel-Haenszel)=5.56, p=0.02,

G allele=susceptibility

TABLE 6
Lymphotoxin alpha (LTA) Thr26Asn A/C polymorphism
allele and genotype frequencies in the ACS
patients and resistant smokers.

| | Allele* | | Genotype | | |
|---|---|---|---|---|---|
| Frequency | A | C | AA | AC | CC |
| ACS | 117 (40%) | 179 (60%) | 16 (11%) | 85 (57%) | 47 (32%) |
| n = 148 (%) | | | | | |
| Resistant | 330 (36%) | 590 (64%) | 60 (13%) | 210 (46%) | 190 (41%) |
| n = 460 (%) | | | | | |

*number of chromosomes (2n)

Genotype. CC vs AA/AC for ACS vs resistant smoker controls, Odds ratio (OR)=0.66, 95% confidence limits 0.4-1.0, χ² (Mantel-Haenszel)=4.28, p=0.04,

CC genotype=protective

LTA Thr26Asn A/C is in linkage disequilibrium with NFKBIL1-63 T/A

TABLE 7
Heat shock protein (HSP) 70 Hom T2437C C/T
polymorphism allele and genotype frequencies
in the ACS patients and resistant smokers.

| | Allele* | | Genotype | | |
|---|---|---|---|---|---|
| Frequency | C | T | CC | CT | TT |
| ACS | 46 (16%) | 248 (84%) | 2 (1%) | 42 (29%) | 103 (70%) |
| n = 147 (%) | | | | | |
| Resistant | 202 (22%) | 710 (78%) | 22 (5%) | 158 (35%) | 276 (61%) |
| n = 456 (%) | | | | | |

*number of chromosomes (2n)

Genotype. CC/CT vs TT for ACS vs resistant smoker controls, Odds ratio (OR)=0.66, 95% confidence limits 0.43-1.0, χ² (Mantel-Haenszel)=4.33, p=0.04,

CC/CT genotype=protective (TT=susceptibility)

Allele C vs T for ACS vs resistant smoker controls, Odds ratio (OR)=0.65, 95% confidence limits 0.5-0.9, χ² (Mantel-Haenszel)=5.75, p=0.02,

C allele=protective

TABLE 8a
Toll like receptor 4 (TLR4) Asp 299Gly A/G
polymorphism allele and genotype frequencies
in the ACS patients and resistant smokers.

| | Allele* | | Genotype | | |
|---|---|---|---|---|---|
| Frequency | A | G | AA | AG | GG |
| ACS | 279 (96%) | 13 (4%) | 135 (92%) | 9 (6%) | 2 (1%) |
| n = 146 (%) | | | | | |
| Resistant | 858 (93%) | 60 (7%) | 399 (87%) | 60 (13%) | 0 (0%) |
| n = 459 (%) | | | | | |

*number of chromosomes (2n)

Genotype. AG/GG vs AA for ACS vs resistant smoker controls, Odds ratio (OR)=0.54, 95% confidence limits 0.3-1.1, χ² (Mantel-Haenszel)=3.27, p=0.07,

AG/GG genotype=protective (AA=susceptibility)

TLR4 Asp299Gly A/G is in linkage disequilibrium with TLR4 Thr399Ile C/T

TABLE 8b
Toll like receptor 4 (TLR4) Thr 399Ile C/T polymorphism allele and
genotype frequencies in the ACS patients and resistant smokers.

| | Allele* | | Genotype | | |
|---|---|---|---|---|---|
| Frequency | C | T | CC | CT | TT |
| ACS | 280 (95%) | 14 (5%) | 135 (92%) | 10 (7%) | 2 (1%) |
| n = 147 (%) | | | | | |
| Resistant | 848 (93%) | 66 (7%) | 392 (86%) | 64 (14%) | 1 (0.2%) |
| n = 457 (%) | | | | | |

*number of chromosomes (2n)

Genotype. CT/TT vs CC for ACS vs resistant smoker controls, Odds ratio (OR)=0.54, 95% confidence limits 0.3-1.1, χ² (Mantel-Haenszel)=3.67, p=0.06,

CT/TT genotype=protective (CC=susceptibility)

TLR4 Thr399Ile C/T is in linkage disequilibrium with TLR4 Asp 299Gly A/G

TABLE 9
Interferon γ (IFNG) 874 A/T polymorphism allele and genotype
frequencies in the ACS patients and resistant smokers.

| | Allele* | | Genotype | | |
|---|---|---|---|---|---|
| Frequency | A | T | AA | AT | TT |
| ACS | 166 (56%) | 128 (44%) | 42 (29%) | 82 (56%) | 23 (16%) |
| n = 147 (%) | | | | | |
| Resistant | 463 (52%) | 427 (48%) | 127 (29%) | 209 (47%) | 109 (24%) |
| n = 445 (%) | | | | | |

*number of chromosomes (2n)

Genotype. TT vs AA/AT for ACS vs resistant smoker controls, Odds ratio (OR)=0.57, 95% confidence limits 0.3-0.96, χ² (Mantel-Haenszel)=4.98, p=0.03,

TT genotype=protective

TABLE 10
Nuclear factor of K light polypeptide gene enhancer in B
cells (NFKBIL1) −63 T/A polymorphism allele and genotype
frequencies in the ACS patients and resistant smokers.

| | Allele* | | Genotype | | |
|---|---|---|---|---|---|
| Frequency | A | T | AA | AT | TT |
| ACS | 184 (63%) | 110 (37%) | 52 (35%) | 80 (54%) | 15 (10%) |
| n = 147 (%) | | | | | |
| Resistant | 584 (66%) | 304 (34%) | 191 (43%) | 202 (46%) | 51 (11%) |
| n = 444 (%) | | | | | |

*number of chromosomes (2n)

Genotype. AA vs AT/TT for ACS vs resistant smoker controls, Odds ratio (OR)=0.73, 95% confidence limits 0.5-1.1, χ² (Mantel-Haenszel)=2.66, p=0.10,

AA genotype=protective

NFKBIL1-63 T/A is in linkage disequilibrium with LTA Thr26Asn

TABLE 11
Platelet derived growth factor receptor alpha (PDGFRA) −1630
insertion/deletion) AACTT/Del polymorphism allele and genotype
frequencies in the ACS patients and resistant smokers.

| | Allele* | | Genotype | | |
|---|---|---|---|---|---|
| Frequency | I | D | II | ID | DD |
| ACS | 239 (81%) | 57 (19%) | 98 (66%) | 43 (29%) | 7 (5%) |
| n = 148 (%) | | | | | |
| Resistant | 702 (76%) | 216 (24%) | 263 (57%) | 176 (38%) | 20 (4%) |
| n = 459 (%) | | | | | |

*number of chromosomes (2n): I = insertion AACTT, D = deletion

Genotype. ID/DD vs II for ACS vs resistant smoker controls, Odds ratio (OR)=0.68, 95% confidence limits 0.5-1.0, χ² (Mantel-Haenszel)=3.69, p=0.05,

ID/DD genotype=protective (II susceptibility)

TABLE 12
Interleukin 4 (IL-4) −589 C/T polymorphism allele and genotype
frequencies in the ACS patients and resistant smokers.

| | Allele* | | Genotype | | |
|---|---|---|---|---|---|
| Frequency | C | T | CC | CT | TT |
| ACS | 264 (90%) | 28 (10%) | 119 (82%) | 26 (18%) | 1 (1%) |
| n = 146 (%) | | | | | |
| Resistant | 792 (87%) | 122 (13%) | 343 (75%) | 106 (23%) | 8 (2%) |
| n = 457 (%) | | | | | |

*number of chromosomes (2n)

Genotype. CT/TT vs CC for ACS vs resistant smoker controls, Odds ratio (OR)=0.68, 95% confidence limits 0.42-1.1, χ² (Mantel-Haenszel)=2.57, p=0.11,

CT/TT genotype=protective (CC=susceptibility)

TABLE 13
Matrix metalloproteinase 1 (MMP1) −1607 1G/2G
polymorphism allele and genotype frequencies in
the ACS patients and resistant smokers.

| | Allele* | | Genotype | | |
|---|---|---|---|---|---|
| Frequency | A | G | 1G1G | 1G2G | 2G2G |
| ACS | 162 (55%) | 132 (45%) | 49 (33%) | 64 (44%) | 34 (23%) |
| n = 147 (%) | | | | | |
| Resistant | 460 (52%) | 426 (48%) | 118 (27%) | 224 (51%) | 101 (23%) |
| n = 443 (%) | | | | | |

*number of chromosomes (2n)

Genotype. 1G1G vs 1G2G/2G2G for ACS vs resistant smoker controls, Odds ratio (OR)=1.4, 95% confidence limits 0.9-2.1, χ² (Mantel-Haenszel)=2.44, p=0.12,

1G1G genotype=susceptibility

TABLE 14
Platelet derived growth factor alpha (PDGFA) 12
IN 5 C/T polymorphism allele and genotype frequencies
in the ACS patients and resistant smokers.

| | Allele* | | Genotype | | |
|---|---|---|---|---|---|
| Frequency | C | T | CC | CT | TT |
| ACS | 143 (49%) | 147 (51%) | 38 (26%) | 67 (46%) | 40 (28%) |
| n = 145 (%) | | | | | |
| Resistant | 481 (53%) | 435 (47%) | 122 (27%) | 237 (52%) | 99 (22%) |
| n = 458 (%) | | | | | |

*number of chromosomes (2n)

Genotype. TT vs CT/CC for ACS vs resistant smoker controls, Odds ratio (OR)=1.4, 95% confidence limits 0.9-2.2, χ² (Mantel-Haenszel)=2.21, p=0.14,

TT genotype=susceptibility

TABLE 15
Glutamate-cysteine ligase modifier subunit (GCLM) −588
C/T polymorphism allele and genotype frequencies
in the ACS patients and resistant smokers.

| | Allele* | | Genotype | | |
|---|---|---|---|---|---|
| Frequency | C | T | CC | CT | TT |
| ACS | 240 (81%) | 56 (19%) | 95 (64%) | 50 (34%) | 3 (2%) |
| n = 148 (%) | | | | | |
| Resistant | 778 (85%) | 142 (15%) | 326 (71%) | 126 (27%) | 8 (2%) |
| n = 460 (%) | | | | | |

*number of chromosomes (2n)

Genotype. CT/TT vs CC for ACS vs resistant smoker controls, Odds ratio (OR)=1.4, 95% confidence limits 0.9-2.0, χ² (Mantel-Haenszel)=2.34, p=0.13,

CT/TT genotype=susceptibility (CC protective)

TABLE 16
Olfactory receptor analogue OR13G1 Ile132Val
A/G polymorphism allele and genotype frequencies
in the ACS patients and resistant smokers.

| | Allele* | | Genotype | | |
|---|---|---|---|---|---|
| Frequency | A | G | AA | AG | GG |
| ACS | 172 (60%) | 116 (40%) | 51 (35%) | 70 (49%) | 23 (16%) |
| n = 144 (%) | | | | | |
| Resistant | 493 (54%) | 421 (46%) | 132 (29%) | 229 (50%) | 96 (21%) |
| n = 457 (%) | | | | | |

*number of chromosomes (2n)

Genotype. AA vs AG/GG for ACS vs resistant smoker controls, Odds ratio (OR)=1.4, 95% confidence limits 0.9-2.1, χ² (Mantel-Haenszel)=2.20, p=0.14,

AA genotype=susceptibility

TABLE 17
Interleukin-10 (IL-10) −1084 A/G polymorphism allele and
genotype frequencies in the ACS patients and resistant smokers.

| | Allele* | | Genotype | | |
|---|---|---|---|---|---|
| Frequency | A | G | AA | AG | GG |
| ACS | 154 (53%) | 136 (47%) | 42 (29%) | 70 (48%) | 33 (23%) |
| n = 145 (%) | | | | | |
| Resistant | 434 (48%) | 476 (52%) | 108 (24%) | 218 (48%) | 129 (28%) |
| n = 455 (%) | | | | | |

*number of chromosomes (2n)

Genotype. GG vs AA/AG for ACS vs resistant smoker controls, Odds ratio (OR)=0.74, 95% confidence limits 0.5-1.2, χ² (Mantel-Haenszel)=1.74, p=0.19,

GG genotype=protective

TABLE 18
alpha1-antitrypsin S allele Glu 288 Val A/T (M/S)
polymorphism allele and genotype frequencies
in the ACS patients and resistant smokers.

| | Allele* | | Genotype | | |
|---|---|---|---|---|---|
| Frequency | A | T | AA | AT | TT |
| ACS | 271 (93%) | 21 (7%) | 126 (86%) | 19 (13%) | 1 (1%) |
| n = 146 (%) | | | | | |
| Resistant | 871 (95%) | 45 (5%) | 414 (90%) | 43 (9%) | 1 (0.2%) |
| n = 458 (%) | | | | | |

*number of chromosomes (2n)

Genotype. AT/TT vs AA for ACS vs resistant smoker controls, Odds ratio (OR)=1.5, 95% confidence limits 0.8-2.7, χ² (Mantel-Haenszel)=1.95, p=0.16,

AT/TT genotype=susceptibility

TABLE 19
Intracellular adhesion molecule 1(ICAM1) K469E
A/G polymorphism allele and genotype frequencies
in the ACS patients and resistant smokers.

| | Allele* | | Genotype | | |
|---|---|---|---|---|---|
| Frequency | A | G | AA | AG | GG |
| ACS | 166 (56%) | 130 (44%) | 41 (28%) | 84 (57%) | 23 (16%) |
| n = 148 (%) | | | | | |
| Resistant | 529 (59%) | 371 (41%) | 159 (35%) | 211 (47%) | 80 (18%) |
| n = 450 (%) | | | | | |

*number of chromosomes (2n)

Genotype. AA vs AG/GG for ACS vs resistant smoker controls, Odds ratio (OR)=0.70, 95% confidence limits 0.5-1.1, χ² (Mantel-Haenszel)=2.91, p=0.09,

AA genotype=protective

TABLE 20
HLA-B associated transcript 1 (BAT1) −23
C/G polymorphism allele and genotype frequencies
in the ACS patients and resistant smokers.

| | Allele* | | Genotype | | |
|---|---|---|---|---|---|
| Frequency | C | G | CC | CG | GG |
| ACS | 109 (39%) | 173 (61%) | 16 (11%) | 77 (55%) | 48 (34%) |
| n = 141 (%) | | | | | |
| Resistant | 322 (35%) | 586 (65%) | 59 (13%) | 204 (45%) | 191 (42%) |
| n = 454 (%) | | | | | |

*number of chromosomes (2n)

Genotype. GG vs CC/CG for ACS vs resistant smoker controls, Odds ratio (OR)=0.71, 95% confidence limits 0.5-1.1, χ² (Mantel-Haenszel)=2.88, p=0.09,

GG genotype=protective

TABLE 21
Nitric oxide synthase 3 (NOS3) Glu298Asp G/T
polymorphism allele and genotype frequencies
in the ACS patients and resistant smokers.

| | Allele* | | Genotype | | |
|---|---|---|---|---|---|
| Frequency | G | T | GG | GT | TT |
| ACS | 186 (65%) | 102 (35%) | 56 (39%) | 74 (51%) | 14 (10%) |
| n = 144 (%) | | | | | |
| Resistant | 605 (68%) | 287 (32) | 209 (47%) | 187 (42%) | 50 (11%) |
| n = 446 (%) | | | | | |

*number of chromosomes (2n)

Genotype. GG vs GT/TT for ACS vs resistant smoker controls, Odds ratio (OR)=0.72, 95% confidence limits 0.5-1.1, χ² (Mantel-Haenszel)=2.79, p=0.09,

GG genotype=protective

TABLE 22
Superoxide dismutase 3 (SOD3) Arg213Gly C/G
polymorphism allele and genotype frequencies
in the ACS patients and resistant smokers.

| | Allele* | | Genotype | | |
|---|---|---|---|---|---|
| Frequency | C | G | CC | CG | GG |
| ACS | 291 (100%) | 1 (0.3%) | 145 (99%) | 1 (1%) | 0 (0%) |
| n = 146 (%) | | | | | |
| Resistant | 892 (98%) | 14 (2%) | 440 (97%) | 12 (3%) | 1 (0.2%) |
| n = 453 (%) | | | | | |

*number of chromosomes (2n)

Genotype. CG/GG vs CC for ACS vs resistant smoker controls, Odds ratio (OR)=0.23, 95% confidence limits 0.01-1.7, χ² (Mantel-Haenszel)=2.31, p=0.13,

CG/GG genotype=protective

TABLE 23
Plasminogen activator inhibitor 1 (PAI-1) −668^{#} Del/G
(4G/5G) polymorphism allele and genotype frequencies in the
ACS patients and resistant smokers.

| | Allele* | | Genotype | | |
|---|---|---|---|---|---|
| Frequency | Del | G | Del.Del | Del.G | GG |
| ACS | 176 (59%) | 120 (41%) | 53 (36%) | 70 (47%) | 25 (17%) |
| n = 148 (%) | | | | | |
| Resistant | 501 (55%) | 403 (45%) | 148 (33%) | 205 (45%) | 99 (22%) |
| n = 452 (%) | | | | | |

*number of chromosomes (2n)
^{#}same as the PAI-1 −675 4G/5G polymorphism

#same as the PAI-1-675 4G/5G polymorphism
Genotype. 5G5G vs 4G5G/4G4G for ACS vs resistant smoker controls, Odds ratio (OR)=0.72, 95% confidence limits 0.4-1.2, χ² (Mantel-Haenszel)=1.7, p=0.19,

5G5G genotype=protective

TABLE 24
Macrophage inflammatory protein 1-alpha (MIP1A)
459 C/T polymorphism allele and genotype frequencies
in the ACS patients and resistant smokers.

| | Allele* | | Genotype | | |
|---|---|---|---|---|---|
| Frequency | C | T | CC | CT | TT |
| ACS | 218 (77%) | 64 (23%) | 81 (57%) | 56 (40%) | 4 (3%) |
| n = 141 (%) | | | | | |
| Resistant | 688 (82%) | 152 (18%) | 268 (64%) | 152 (36%) | 0 (0%) |
| n = 420 (%) | | | | | |

*number of chromosomes (2n)

Genotype. CT/TT vs CC for ACS vs resistant smoker controls, Odds ratio (OR)=1.31, 95% confidence limits 0.9-2.0, χ² (Mantel-Haenszel)=1.81, p=0.18,

CT/TT genotype=susceptibility

Allele. T vs C for ACS vs resistant smoker controls, Odds ratio (OR)=1.33, 95% confidence limits 0.9-1.9, χ² (Mantel-Haenszel)=2.87, p=0.09,

T allele=susceptibility

TABLE 25
Matrix metalloproteinase 7 (MMP7) −181 A/G
polymorphism allele and genotype frequencies
in the ACS patients and resistant smokers.

| | Allele* | | Genotype | | |
|---|---|---|---|---|---|
| Frequency | A | G | AA | AG | GG |
| ACS | 171 (58%) | 125 (42%) | 42 (28%) | 87 (59%) | 19 (13%) |
| n = 148 (%) | | | | | |
| Resistant | 526 (57%) | 392 (43%) | 147 (32%) | 232 (51%) | 80 (17%) |
| n = 459 (%) | | | | | |

*number of chromosomes (2n)

Genotype. GG vs AA/AG for ACS vs resistant smoker controls, Odds ratio (OR)=0.70, 95% confidence limits 0.4-1.2, χ² (Mantel-Haenszel)=1.73, p=0.19,

GG genotype=protective

TABLE 26
Cathepsin G Asn125Ser A/G polymorphism allele and genotype
frequencies in the ACS patients and resistant smokers.

| | Allele* | | Genotype | | |
|---|---|---|---|---|---|
| Frequency | A | G | AA | AG | GG |
| ACS | 279 (96%) | 11 (4%) | 135 (93%) | 9 (6%) | 1 (1%) |
| n = 145 (%) | | | | | |
| Resistant | 846 (94%) | 52 (6%) | 398 (89%) | 50 (11%) | 1 (0.2%) |
| n = 449 (%) | | | | | |

*number of chromosomes (2n)

Genotype. AG/GG vs AA for ACS vs resistant smoker controls, Odds ratio (OR)=0.58, 95% confidence limits=0.27-1.22, χ² (Mantel-Haenszel)=2.36, p=0.12,

AG/GG genotype=protective (AA susceptibility)

TABLE 27
Chemokine (CX3C motif) receptor 1 (CX3CR1) I249V
C/T polymorphism allele and genotype frequencies
in the ACS patients and resistant smokers.

| | Allele* | | Genotype | | |
|---|---|---|---|---|---|
| Frequency | C | T | CC | CT | TT |
| ACS | 201 (69%) | 91 (31%) | 74 (51%) | 53 (36%) | 19 (13%) |
| n = 146 (%) | | | | | |
| Resistant | 651 (71%) | 265 (29%) | 234 (51%) | 183 (40%) | 41 (9%) |
| n = 458 (%) | | | | | |

number of chromosomes (2n)

Genotype. TT vs CC/CT for ACS vs resistant smoker controls, Odds ratio (OR)=1.5, 95% confidence limits=0.82-2.81, χ² (Mantel-Haenszel)=2.04, p=0.15,

TT genotype=susceptibility

TABLE 28
Caspase (NOD2) Gly881Arg G/C polymorphism allele and genotype
frequencies in the ACS patients and resistant smokers.

| | Allele* | | Genotype | | |
|---|---|---|---|---|---|
| Frequency | C | G | CC | CG | GG |
| ACS | 6 (2%) | 288 (98%) | 0 (0%) | 6 (4%) | 141 (96%) |
| n = 147 (%) | | | | | |
| Resistant | 9 (1%) | 905 (99%) | 0 (0%) | 9 (2%) | 448 (98%) |
| n = 457 (%) | | | | | |

*number of chromosomes (2n)

Genotype. CC/CG vs GG for ACS vs resistant smoker controls, Odds ratio (OR)=2.1, 95% confidence limits=0.662-6.6, χ² (Mantel-Haenszel)=2.05, p=0.15,

CC/CG genotype=susceptibility

TABLE 29
Tissue inhibitor of metalloproteinase 1 (TIMP1)
372 T/C polymorphism allele and genotype frequencies
in the ACS patients and resistant smokers.

| | Allele* | | Genotype | | |
|---|---|---|---|---|---|
| Frequency | C | T | CC | CT | TT |
| ACS | 202 (68%) | 94 (32%) | 64 (43%) | 74 (50%) | 10 (7%) |
| n = 148 (%) | | | | | |
| Resistant | 517 (57%) | 397 (43%) | 158 (35%) | 201 (44%) | 98 (21%) |
| n = 457 (%) | | | | | |

number of chromosomes (2n)

Genotype. TT vs CT/CC for ACS vs resistant smoker controls, Odds ratio (OR)=0.27, 95% confidence limits=0.13-0.54, χ² (Mantel-Haenszel)=16.42, p=0.00005,

TT genotype=protective

Genotype. CC vs CT/TT for ACS vs resistant smoker controls, Odds ratio (OR)=1.4, 95% confidence limits=1.0-2.1, χ² (Mantel-Haenszel)=3.61, p=0.06,

CC genotype=susceptibility

Allele T vs C, ACS vs resistant smoker controls, Odds ratio (OR)=0.61, 95% confidence limits=0.45-0.81, χ² (Mantel-Haenszel)=12.64, p=0.0004,

T allele=protective

Table 30 below presents a summary of the protective and susceptibility SNPs identified herein. Selected susceptibility SNPs are identified as S1 through S13, while selected protective SNPs are identified as P1 through P16. Those shown in bold were included in panels of SNPs used to generate a SNP score as discussed below.

TABLE 30
Summary of Protective and susceptibility SNPs for Acute Coronary Syndrome

| Gene | rs | Polymorphism | Genotype | SNP# | Phenotype | OR | P value |
|---|---|---|---|---|---|---|---|
| CMA1 | 1800875 | −1903 A/G | GG | S1 | susceptibility | 1.9 | 0.004 |
| TGFB1 | 1800469 | −509 C/T | CC | S2 | susceptibility | 1.5 | 0.05 |
| MMP12 | 2276109 | −82 A/G | GG | S3 | susceptibility | 3.2 | 0.05 |
| FGF2 | 1449683 | Ser52Ser 223 C/T | CT/TT | S4 | susceptibility | 1.5 | 0.08 |
| | | | (CC) | | (protective) | | |
| IL4RA | 1801275 | Q576R A/G | GG | S5 | susceptibility | 2.7 | 0.02 |
| | | | AA | | protective | 0.47 | 0.05 |
| LTA | 1041981 | Thr26Asn A/C | CC | P1 | protective | 0.66 | 0.04 |
| HSP70 | 2227956 | Hom T2437C | CC/CT | P2 | protective | 0.66 | 0.04 |
| | | | (TT) | | (susceptibility) | | |
| TLR4 | 4986790 | ¹Asp299Gly A/G | AG/GG | P3 | protective | 0.54 | 0.07 |
| | | | (AA) | | (susceptibility) | | |
| TLR4 | 4986791 | ²Thr399Ile C/T | CT/TT | P3.1 | protective | 0.54 | 0.06 |
| | | | (CC) | | (susceptibility) | | |
| IFNG | 2430561 | 874 A/T | TT | P4 | protective | 0.57 | 0.03 |
| NFKBIL1 | 2071592 | −63 T/A | AA | P11 | protective | 0.73 | 0.10 |
| PDGFRA | — | −1630 I/D, | I/Del, Del/Del | P5 | protective | 0.68 | 0.05 |
| | | (AACTT/Del) | (II) | | (susceptibility) | | |
| IL4 | 2243250 | −589 C/T | CT/TT | P6 | protective | 0.68 | 0.11 |
| | | | (CC) | | (susceptibility) | | |
| MMP1 | 1799750 | −1607 1G/2G (Del/G) | Del.Del (ie | S6 | susceptibility | 1.4 | 0.12 |
| | | | 1G1G) | | | | |
| PDGFA | — | 12 IN5 C/T | TT | S7 | susceptibility | 1.4 | 0.14 |
| GCLM | — | −588 C/T | CT/TT | S8 | susceptibility | 1.4 | 0.13 |
| | | | (CC) | | (protective) | | |
| OR13G1 | 1151640 | Ile132Val A/G | AA | S9 | susceptibility | 1.4 | 0.14 |
| IL-10 | 1800896 | −1084 A/G (−1082) | GG | P12 | protective | 0.74 | 0.19 |
| α1-AT S | 17580 | Glu288Val A/T | AT/TT | S10 | susceptibility | 1.5 | 0.16 |
| allele | | (M/S) | (MS/SS) | | | | |
| ICAM1 | 5498 | K469E A/G | AA | P7 | protective | 0.70 | 0.09 |
| BAT1 | 2239527 | −23 C/G | GG | P8 | protective | 0.71 | 0.09 |
| NOS3 | 1799983 | Glu298Asp G/T | GG | P9 | protective | 0.72 | 0.09 |
| SOD3 | 1799895 | Arg213Gly C/G | CG/GG | P10 | protective | 0.23 | 0.13 |
| PAI-1 | — | −668 4G/5G | 5G5G | P13 | protective | 0.72 | 0.19 |
| MIP1A | 1719134 | +459 C/T Intron 1 | CT/TT | S11 | susceptibility | 1.31 | 0.18 |
| MMP7 | 17880821 | −181 A/G | GG | P14 | protective | 0.70 | 0.19 |
| Cathepsin G | | Asn 125Ser | AG/GG | P15 | protective | 0.58 | 0.12 |
| | | | AA | | (susceptibility) | | |
| CX3CR1 | 3732379 | I249V | TT | S12 | susceptibility | 1.5 | 0.15 |
| NOD2 | 2066845 | Gly 881 Arg G/C | CC/CG | S13 | susceptibility | 2.1 | 0.15 |
| TIMP1 | 4898 | 372 T/C | TT | P16 | protective | 0.27 | 0.00005 |
| | | | CC | | susceptibility | 1.4 | 0.06 |

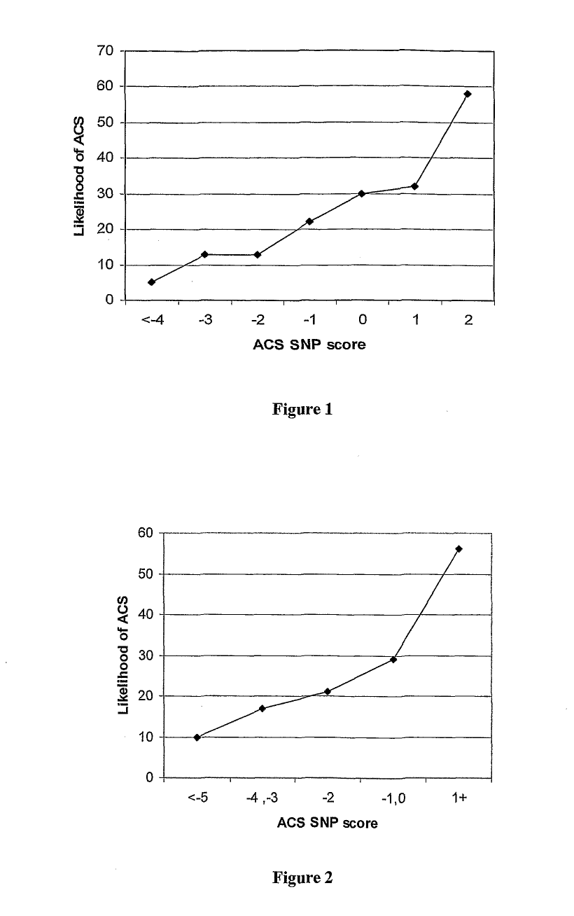

As discussed herein, S3 is in LD with S6, P1 is in LD with P11 and P3 is in LD with P3.1. Hence, these SNPs were not used together in a panel when deriving the SNP score. Table 31 below shows the distribution of ACS patients and smoking controls with reference to a SNP score. The SNP score for each individual was determined in a combined analysis of an 11 SNP panel consisting of SNPs S1-S5 and P1-P6 as shown in Table 30. Each susceptibility SNP was assigned a value of +1, and each protective SNP was assigned a value of −1. FIG. 1 presents this data graphically.

TABLE 31
Distribution of those with ACS according to SNP score - 11 SNP panel.

| | SNP score based on 11 SNP panel | | | | | | | |
|---|---|---|---|---|---|---|---|---|
| Cohort | <−4 | −3 | −2 | −1 | 0 | 1 | 2+ | total |
| Smoking controls | 18 | 53 | 88 | 129 | 107 | 51 | 14 | 460 |
| ACS | 1 | 8 | 13 | 37 | 46 | 24 | 19 | 148 |
| % with ACS | 1/19 | 8/61 | 13/101 | 37/166 | 46/153 | 24/75 | 19/33 | 608 |
| | 5% | 13% | 13% | 22% | 30% | 32% | 58% | |

Table 32 below shows the distribution of ACS patients and smoking controls according to the SNP score determined with reference to a larger, 15 SNP, panel. This 15 SNP panel consisted of SNPs S1-S5 and P1-P10 as shown in Table 30. Again, each susceptibility SNP was assigned a value of +1, and each protective SNP was assigned a value of −1. FIG. 2 presents the data shown in Table 32 graphically.

TABLE 32
Distribution of those with ACS according to SNP score - 15 SNP panel.

| | SNP score based on 15 SNP panel | | | | | | | | | |
|---|---|---|---|---|---|---|---|---|---|---|
| Cohort | <−6 | −5 | −4 | −3 | −2 | −1 | 0 | 1 | 2+ | Total |
| Smoking controls | 22 | 35 | 55 | 84 | 98 | 83 | 60 | 21 | 2 | 460 |
| ACS | 0 | 6 | 12 | 16 | 26 | 38 | 21 | 18 | 11 | 148 |
| % with ACS | 0/22 | 6/41 | 12/67 | 16/100 | 26/124 | 38/121 | 21/81 | 18/39 | 11/13 | 608 |
| | 0% | 15% | 18% | 16% | 21% | 31% | 26% | 46% | 84% | |

| | | | | | |
|---|---|---|---|---|---|
| % with ACS (by grouping | 6/63 | 28/167 | 26/124 | 49/202 | 29/52 |
| comparable risk scores) | 10% | 17% | 21% | 29% | 56% |

### Discussion

The above results show that several polymorphisms were associated with either increased or decreased risk of developing ACS. The associations of individual polymorphisms on their own, while of discriminatory value, are unlikely to offer an acceptable prediction of disease. However, in combination these polymorphisms distinguish susceptible subjects from those who are resistant (for example, between the smokers who develop ACS and those with the least risk with comparable smoking exposure). The polymorphisms represent both promoter polymorphisms, thought to modify gene expression and hence protein synthesis, and exonic polymorphisms known to alter amino-acid sequence (and likely expression and/or function) in a number of genes encoding proteins central to processes including inflammation, matrix remodelling, and cytokine activity.

In the comparison of smokers with ACS and matched smokers without ACS (lowest risk for ACS despite smoking), several polymorphisms were identified as being found in significantly greater or lesser frequency than in the comparator group. Due to the small cohort of ACS patients, polymorphisms where there are only trends towards differences (P=0.06-0.25) were included in the analyses, although in the combined analyses only those polymorphisms with the most significant differences were utilised.
In the analysis of the −1903 A/G polymorphism of the Chymase 1 gene, the GG genotype was found to be greater in the ACS cohort (OR=1.9, P=0.004) consistent with a susceptibility role (see Table 1). The G allele was also found to be significantly greater in the ACS cohort compared to the resistant smoking cohort (OR=1.4, p=0.01) consistent with a susceptibility role (Table 1).
In the analysis of the −509 C/T in the TGFB1 gene, the CC genotype was found to be greater in the ACS cohort compared to the resistant smoker cohort (OR=1.5, p=0.05) consistent with a susceptibility role (see Table 2). The C allele was also found to be significantly greater in the ACS cohort compared to the resistant smoker cohort (OR=1.4, p=0.05) consistent with a susceptibility role (Table 2).
In the analysis of the −82 A/G polymorphism in the MMP12 gene, the GG genotype was found to be greater in the ACS cohort compared to the resistant smoker cohort (OR=3.2, p=0.05) consistent with a susceptibility role (see Table 3).
In the analysis of the Ser52Ser (223 C/T) polymorphism of the FGF2 gene, the CT and TT genotypes were found to be greater in the ACS cohort compared to the resistant smoker cohort (OR=1.5, p=0.08) consistent with each having a susceptibility role. The T allele was found to be greater in the ACS cohort compared to the resistant smoker cohort (OR=1.5, p=0.07) consistent with a susceptibility role. In contrast, the CC genotype was found to be consistent with a protective role (Table 4).
In the analysis of the Q576R A/G polymorphism of the IL4RA gene, the GG genotype was found to be greater in the ACS cohort compared to the resistant smoker cohort (OR=2.71, p=0.02) consistent with a susceptibility role (see Table 5). The G allele was also found to be significantly greater in the ACS cohort compared to the resistant smoker cohort (OR=1.5, p=0.02) consistent with a susceptibility role (Table 5). In contrast the AA genotype was found to be greater in the resistant smoker cohort compared to the ACS cohort (OR=0.47, p=0.05) consistent with a protective role (see Table 5).
In the analysis of the Thr26Asn A/C polymorphism of the LTA gene, the CC genotype was found to be greater in the resistant smoker cohort compared to the ASC cohort (OR=0.66, p=0.04) consistent with a protective role (see Table 6).
In the analysis of the HOM T2437C C/T polymorphism of the HSP 70 gene, the CC and CT genotypes were found to be greater in the resistant smoker cohort compared to the ACS cohort (OR=0.66, p=0.04) consistent with each having a protective role (see Table 7). The C allele was also found to be greater in the resistant smoker cohort compared to the ACS cohort (OR=0.65, p=0.02) consistent with a protective role. In contrast the TT genotype was found to be consistent with a susceptibility role (see Table 7).
In the analysis of the Asp299Gly A/G polymorphism of the TLR4 gene, the AG and GG genotypes were found to be greater in the resistant smoker cohort compared to the ACS cohort (OR=0.54, p=0.07) consistent with each having a protective role (see Table 8a). In contrast, the AA genotype was found to be consistent with a susceptibility role (see Table 8a).
In the analysis of the Thr399Ile C/T polymorphism of the TLR4 gene, the CT and TT genotypes were found to be greater in the resistant smoker cohort compared to the ACS cohort (OR=0.54, p=0.06) consistent with each having a protective role (see Table 8b). In contrast the CC genotype was found to be consistent with a susceptibility role (Table 8b).
In the analysis of the 874 A/T polymorphism of the IFNG gene, the TT genotype was found to be greater in the resistant smoker cohort compared to the ACS cohort (OR=0.57, p=0.03) consistent with a protective role (see Table 9).
In the analysis of the −63 T/A polymorphism of the NFKBIL1 gene, the AA genotype was found to be greater in the resistant smoker cohort compared to the ACS cohort (OR=0.73, p=0.10) consistent with a protective role (see Table 10).
In the analysis of the −1630 Ins/Del (AACTT/Del) polymorphism of the PDGFRA gene, the Ins Del and Del Del genotypes were found to be greater in the resistant smoker cohort compared to the ACS cohort (OR=0.68, p=0.05) consistent with each having a protective role (Table 11). In contrast the Ins Ins was found to be consistent with a susceptibility role (see Table 11).
In the analysis of the −589 C/T polymorphism of the IL-4 gene, the CT and TT genotypes were found to be greater in the resistant smoker cohort compared to the ACS cohort (OR=0.68, p=0.11) consistent with each having a protective role (see Table 12). In contrast the CC genotype was found to be consistent with a susceptibility role (Table 12).
In the analysis of the −1607 1G/2G polymorphism of the MMP1 gene, the 1G1G genotype was found to be greater in the ACS cohort compared to the resistant smoker cohort (OR=1.4, p=0.12) consistent with a susceptibility role (see Table 13).
In the 12 IN 5 C/T polymorphism of the PDGFA gene, the TT genotype was found to be greater in the ACS cohort compared to the resistant smoker cohort (OR=1.4, p=0.14) consistent with a susceptibility role (see Table 14).
In the −588 C/T polymorphism in the GCLM gene, the CT and TT genotypes were found to be greater in the ACS cohort compared to the resistant smoker cohort (OR=1.4, p=0.13) consistent with each having a susceptibility role (see Table 15). In contrast, the CC genotype was found to be consistent with a protective role (see Table 15).
In the Ile32Val A/G polymorphism of the OR13G1 gene, the AA genotype was found to be greater in the ACS cohort compared to the resistant smoker cohort (OR=1.4, p=0.14) consistent with a susceptibility role (Table 16).
In the analysis of the −1084 A/G polymorphism of the 11-10 gene, the GG genotype was found to be greater in the resistant smoker cohort compared to the ACS cohort (OR=0.74, p=0.19) consistent with a protective role (see Table 17).
In the analysis of the Glu288Val A/T (M/S) polymorphism in the α1-AT gene, the AT and TT genotypes were found to be greater in the ACS cohort compared to the resistant smoker cohort (OR=1.5, p=0.16) consistent with each having a susceptibility role (see Table 18).
In the K469E A/G polymorphism in the ICAM1 gene, the AA genotype was found to be greater in the resistant smoker cohort compared to the ACS cohort (OR=0.70, p=0.09) consistent with a protective role (see Table 19).
In the analysis of the −23 C/G polymorphism of the BAT1 gene, the GG genotype was found to be greater in the resistant smoker cohort compared to the ACS cohort (OR=0.71, p=0.09) consistent with a protective role (see Table 20).
In the analysis of the Glu298Asp (G/T) polymorphism of the Nitric oxide synthase 3 gene, the GG genotype was found to be greater in the smoking resistant cohort compared to the ACS cohort (OR=0.72, p=0.09) consistent with a protective role (see Table 21).
In the analysis of the Arg213Gly C/G polymorphism of the SOD3 gene, the CG and GG genotypes were found to be greater in the resistant smoker cohort compared to the ACS cohort (OR=0.23, p=0.13) consistent with each having a protective role (see Table 22).
In the analysis of the −668 Del/G (4G/5G) polymorphism of the PAI-1 gene, the 5G5G genotype was found to be greater in the resistant smoker cohort compared to the ACS cohort (OR=0.72, p=0.19) consistent with a protective role (see Table 23).
In the analysis of the 459 C/T polymorphism of the MIP1A gene, the CT and TT genotypes were found to be greater in the ACS cohort compared to the resistant smoker cohort (OR=1.31, p=0.18) consistent with each having a susceptibility role (see Table 24). The T allele was also found to be greater in the ACS cohort compared to the resistant smoker cohort (OR=1.33, p=0.09) consistent with a susceptibility role (Table 24).
In the analysis of the −181 A/G polymorphism of the MMP7 gene, the GG genotype was found to be greater in the resistant smoker cohort compared to the ACS cohort (OR=0.70, p=0.19) consistent with a protective role (see Table 25).
In the analysis of the Asn125Ser A/G polymorphism of the Cathespin G gene, the AG and GG genotypes were found to be greater in the resistant smoker cohort compared to the ACS cohort (OR=0.58, p=0.12) consistent with each having a protective role (see Table 26). In contrast the AA genotype was found to be consistent with a susceptibility role (Table 26).
In the I249V C/T polymorphism of the CX3CR1 gene, the TT genotype was found to be greater in the ACS cohort compared to the resistant smoker cohort (OR=1.5, p=0.15) consistent with a susceptibility role (Table 27).
In the analysis of the Gly881Arg G/C polymorphism in the NOD2 gene, the CC and CG genotypes were found to be greater in the ACS cohort compared to the resistant smoker cohort (OR=2.1, p=0.15) consistent with each having a susceptibility role (see Table 28).
In the analysis of the 372 T/C polymorphism of the TIMP1 gene, the TT genotype was found to be greater in the resistant smoker cohort compared to the ACS cohort (OR=0.27, p=0.00005) consistent with a protective role (see Table 29). The T allele was also found to be significantly greater in the resistant smoker cohort compared to the ACS cohort (OR=0.61, p=0.0004) consistent with a protective role (Table 29). In contrast the CC genotype was found to be greater in the ACS cohort compared to the resistant smoker cohort (OR=1.4, p=0.06) consistent with a susceptibility role (see Table 29).

It is accepted that the disposition to ACS is the result of the combined effects of the individual's genetic makeup and other factors, including their lifetime exposure to various aero-pollutants including tobacco smoke. Similarly, it is accepted that ACS encompasses several vascular diseases. The data herein suggest that several genes can contribute to the development of ACS. A number of genetic mutations working in combination either promoting or protecting the vasculature from damage are likely to be involved in elevated resistance or susceptibility to ACS.

From the analyses of the individual polymorphisms, 20 susceptibility genotypes and 20 protective genotypes were identified and analysed for their frequencies in the smoker cohort consisting of resistant smokers and those with ACS. In a pre-defined algorithm, where the presence of a susceptibility genotype scores +1 and the presence of a protective genotype scores −1, an ACS SNP score has been generated for each subject. The ACS SNP score generated with reference to an 11 SNP panel is linearly related to the frequency of having ACS. For example, the chance of having ACS diminished from 58% in smokers with a SNP score of +2 to 5% in smokers with a SNP score of −4 or less (see Table 31 and FIG. 1). In a further analysis with a 15 SNP panel consisting of the SNPs S1-S5 and P1-P10 as shown in Table 30, the chance of having ACS diminished from 84% in smokers with a SNP score of 2 or greater, to 0% in smokers with a SNP score of −6 or less (see Table 32 and FIG. 2).

Furthermore, the log odds of having ACS is linearly related to the ACS SNP score—the greater the SNP score, the greater likelihood of having an acute coronary syndrome (see FIG. 3). From preliminary analyses of the C statistic (equivalent to the area under the curve of a receiver operator curve that optimises sensitivity and specificity of a test), the C statistic values are as follows: SNP score alone=0.65, SNP score/age=0.74, SNP score/BMI/gender=0.78 and SNP score/BMI/gender/age=0.93.

Thus, the ACS SNP score is independently associated with having ACS and can be used alone or in conjunction with non-genetic risk factors to assess risk of ACS and of having an acute coronary event.

These findings indicate that the methods of the present invention may be predictive of ACS in an individual well before symptoms present.

These findings therefore also present opportunities for therapeutic interventions and/or treatment regimens, as discussed herein. Briefly, such interventions or regimens can include the provision to the subject of motivation to implement a lifestyle change, or therapeutic methods directed at normalising aberrant gene expression or gene product function. For example, as shown herein the −675 5G5G genotype in the promoter of the PAI-1 gene is associated with decreased risk of developing ACS. The 5G allele is reportedly associated with increased binding of a repressor protein and decreased transcription of the gene. A suitable therapy for individuals having the −675 4G4G genotype can be the administration of an agent capable of increasing the level of repressor and/or enhancing binding of the repressor, thereby augmenting its downregulatory effect on transcription. An alternative therapy can include gene therapy, for example the introduction of at least one additional copy of a gene encoding a repressor having an increased affinity for binding a PAI-1 gene having a −675 4G4G genotype. In a further example, as shown herein the −82 A/G GG genotype in the promoter of the gene encoding MMP12 is associated with susceptibility to ACS. A number of inhibitors of matrix metalloproteinases are known, for example those discussed in U.S. Pat. No. 6,600,057 (incorporated herein in its entirety), such as tissue inhibitors of metalloproteinases (TIMPs) including TIMP1, TIMP2, TIMP3, and TIMP4, which form inactive complexes with MMPs, more general proteinase regulators which prevent MMP action, regulators of MMP gene expression including membrane bound MMPs (MT-MMP) that activate the excreted proenzyme form of MMPs, and compounds such as 4,5-dihydroxyanthaquinone-2-carboxylic acid (AQCA) and derivatives thereof. A suitable therapy in subjects known to possess the −82 A/G GG genotype can be the administration of an agent capable of reducing expression of the gene encoding MMP12, or administration of an agent capable of reducing the activity of MMP12, for example by administration of an agent capable of increasing expression of or the activity of one or more TIMPs, or administration of an agent capable of reducing expression of or the activity of one or more membrane bound MMPs or other activators of MMP12. For example, a suitable therapy can be the administration to such a subject of a MMP1 inhibitor such as 4,5-dihydroxyanthaquinone-2-carboxylic acid (AQCA), anthraquinyl-mercaptoethyamine, anthraquinyl-alanine hydroxamate, or derivatives thereof. Similarly, the 372 T/C CC genotype in the gene encoding TIMP1 is associated with susceptibility to ACS. A suitable therapy in subjects known to possess the 372 T/C CC genotype can be the administration of an agent capable of modulating, and preferably increasing, the expression of the gene encoding TIMP1.

In another example; a given susceptibility genotype is associated with increased expression of a gene relative to that observed with the protective genotype. A suitable therapy in subjects known to possess the susceptibility genotype is the administration of an agent capable of reducing expression of the gene, for example using antisense or RNAi methods. An alternative suitable therapy can be the administration to such a subject of an inhibitor of the gene product. In still another example, a susceptibility genotype present in the promoter of a gene is associated with increased binding of a repressor protein and decreased transcription of the gene. A suitable therapy is the administration of an agent capable of decreasing the level of repressor and/or preventing binding of the repressor, thereby alleviating its downregulatory effect on transcription. An alternative therapy can include gene therapy, for example the introduction of at least one additional copy of the gene having a reduced affinity for repressor binding (for example, a gene copy having a protective genotype).

Suitable methods and agents for use in such therapy are well known in the art, and are discussed herein.

The identification of both susceptibility and protective polymorphisms as described herein also provides the opportunity to screen candidate compounds to assess their efficacy in methods of prophylactic and/or therapeutic treatment. Such screening methods involve identifying which of a range of candidate compounds have the ability to reverse or counteract a genotypic or phenotypic effect of a susceptibility polymorphism, or the ability to mimic or replicate a genotypic or phenotypic effect of a protective polymorphism.

Still further, methods for assessing the likely responsiveness of a subject to an available prophylactic or therapeutic approach are provided. Such methods have particular application where the available treatment approach involves restoring the physiologically active concentration of a product of an expressed gene from either an excess or deficit to be within a range which is normal for the age and sex of the subject. In such cases, the method comprises the detection of the presence or absence of a susceptibility polymorphism which when present either upregulates or downregulates expression of the gene such that a state of such excess or deficit is the outcome, with those subjects in which the polymorphism is present being likely responders to treatment.

### Example 2

This example describes the substitution of SNPs identified herein as being associated with risk of ACS with SNPs in linkage disequilibrium, and shows that such SNPs can have comparable utility in deriving a SNP score. Here, alternative SNPs can be used to derive a SNP score when SNPs in LD are substituted for the specific SNPs recited herein. To illustrate this, the TLR4 Asp299Gly A/G SNP was substituted with the TLR4 Thr399Ile C/T SNP in the 11 SNP panel. These two SNPs are reported to be in linkage disequilibrium (the G allele of the Asp299Gly polymorphism reportedly nearly always cosegregates with the T allele of the Thr399Ile polymorphism). This cosegregation is clearly shown in Table 33 below, where there is 99% concordance between the genotypes of the Asp299Gly polymorphism and Thr399Ile polymorphism (genotyping “fails” are excluded).

TABLE 33
Concordance between the TLR4 299 and 399 polymorphisms.

| TLR4 299 genotype | TLR4 399 genotype | Concordance (%) | Totals |
|---|---|---|---|
| AA 534 | CC 524 | 524/530 | 534 |
| | TC 6 | (99%) | |
| | Fails 4 | | |
| AG 69 | TC 68 | 68/69 | 69 |
| | TT 1 | (99%) | |
| GG 2 | TT 2 | 2/2 | 2 |
| | | (100%) | |
| Fails 3 | CC 3 | | 3 |
| | | | 608 |

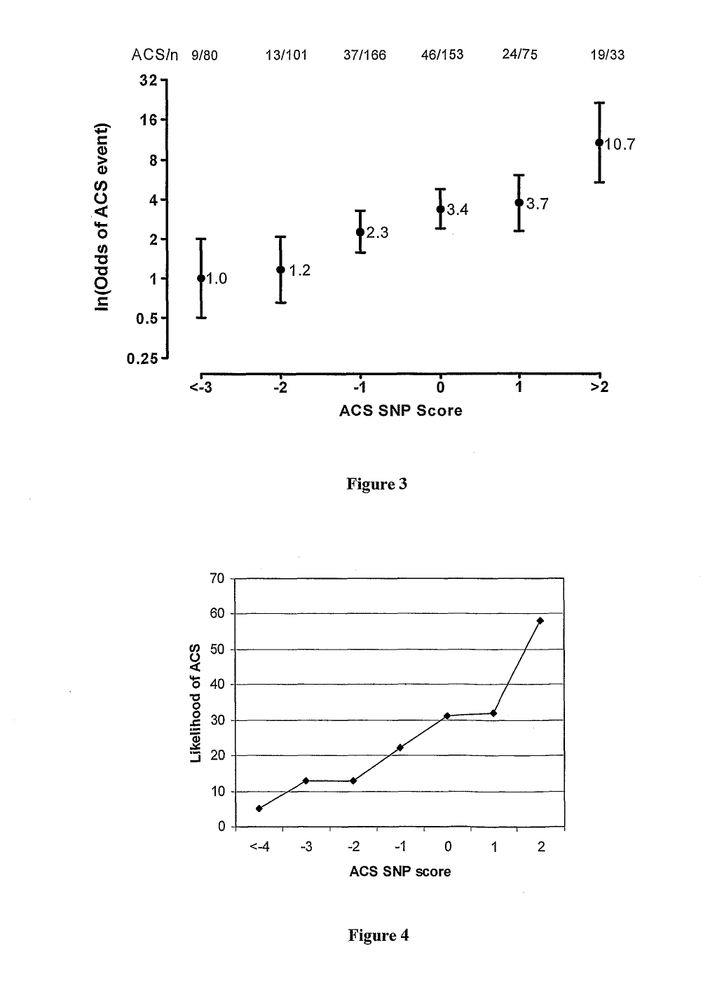

Table 34 shows the distribution of SNP score in the ACS and control groups when the TLR4 Asp299Gly SNP is replaced with the TLR4 Thr399Ile SNP for the 11 SNP panel. In deriving the SNP score this means substituting the score for the AG/GG genotype (protective=+1) with the score for the CT/TT genotype (protective=+1) and calculating the score with the latter. The shaded cells in Table 34 identify differences with respect to the comparable groups shown in Table 31. The graph depicted in FIG. 4 shows the score graphically and is similar to that of FIG. 1.

TABLE 34
Distribution of those with ACS according to
SNP score—substituted 11 SNP panel

| | SNP score based on 11 SNP panel | | | | | | | total |
|---|---|---|---|---|---|---|---|---|
| Cohort | <−4 | −3 | −2 | −1 | 0 | 1 | 2+ | |
| Smoking controls | 18 | 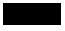 | 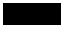 | 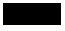 | 107 | 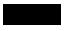 | 14 | 460 |
| ACS | 1 | 8 | 13 | 37 | 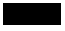 | 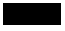 | 19 | 148 |
| % with ACS | 1/19 | 8/62 | 13/102 | 37/165 | 47/154 | 23/73 | 19/33 | 608 |
| | 5% | 13% | 13% | 22% | 31% | 32% | 58% | |

Therefore, SNPs that are in LD with the SNPs used herein to derive a SNP score may be substituted with SNPs in LD to derive a clinically meaningful score.

Table 35 below presents representative examples of polymorphisms in linkage disequilibrium with the polymorphisms specified herein in Table 30. Examples of such polymorphisms can be located using public databases, such as that available at www.hapmap.org. Specified polymorphisms are indicated in parentheses. As those skilled in the art will recognise, the rs numbers provided are identifiers unique to each polymorphism.

These results show that SNPs in LD with the SNPs recited herein, such as those from Table 35, could be utilised in a SNP score with similar clinical utility.

TABLE 35
SNPs in linkage disequilibrium with the SNPs associated
with either a susceptibility or protective phenotype.
CMA1

| | | | | |
|---|---|---|---|---|
| rs7150627 | rs2007323 | rs11844710 | rs7151943 | rs7148705 |
| rs1885107 | rs9806075 | rs4982956 | rs1956920 | rs7146705 |
| rs12434912 | rs1956921 | rs2208447 | rs927281 | rs1800875 |
| | | | | (−1903 A/G) |
| rs7400965 | rs761988 | rs1800876 | rs6573850 | rs1956916 |
| rs3759635 | rs8007723 | rs1956917 | rs1956922 | rs927278 |
| rs1956918 | rs1956923 | rs2007316 | rs1956919 | |

TGFB1

| | | | |
|---|---|---|---|
| rs1529717 | rs13447341 | rs1046909 | rs2241712 |
| rs2241714 | rs1982072 | rs2317130 | rs4803457 |
| rs1800469 | rs1982073 | rs1800471 | |
| (−509 C/T) | | | |

MMP12

| | | | | |
|---|---|---|---|---|
| rs17368890 | rs11225444 | rs608194 | rs12793969 | rs1277718 |
| rs4323842 | rs17099726 | rs7931510 | rs12786343 | rs7934568 |
| rs17099720 | rs4420231 | rs7934710 | rs3814766 | rs4393288 |
| rs621170 | rs2276109 | rs11225443 | rs11225445 | rs10895367 |
| | (−82 A/G) | | | |
| rs17368659 | rs636648 | rs495914 | rs7126998 | rs17368814 |
| rs610341 | rs1799750 | rs501371 | rs610338 | |
| | (MMP1 −1607 G/GG) | | | |

FGF2

| | | | | |
|---|---|---|---|---|
| rs13434728 | rs308395 | rs12509901 | rs308405 | rs13434738 |
| rs308396 | rs177814 | rs308404 | rs308447 | rs308397 |
| rs308412 | rs10470916 | rs13435470 | rs308398 | rs1563704 |
| rs308403 | rs10003693 | rs308399 | rs10049563 | rs308402 |
| rs11940812 | rs308400 | rs308411 | rs308401 | rs13137174 |
| rs1449683 | rs308410 | rs10028307 | rs2922979 | rs416124 |
| (Ser52Ser) | | | | |
| rs7668232 | rs3804144 | rs374880 | rs13349352 | rs2922980 |
| rs308409 | rs11940156 | rs12650248 | rs4574429 | rs308391 |
| rs1992980 | rs308408 | rs12507250 | rs2922981 | rs367344 |
| rs308392 | rs1984971 | rs367331 | rs11098673 | rs366192 |
| rs308407 | rs6828763 | rs411027 | rs968346 | rs6829351 |
| rs421184 | rs4254785 | rs308393 | rs2926173 | rs728694 |
| rs6830094 | rs11934448 | rs13119363 | rs308394 | rs4146526 |
| rs308406 | | | | |

IL4RA

| | | | | |
|---|---|---|---|---|
| rs3024672 | rs1805012 | rs3024696 | rs2234899 | rs3024673 |
| rs2234923 | rs3024674 | rs2234900 | rs3024675 | rs1805013 |
| rs3024676 | rs1805015 | rs2234897 | rs1801275 | rs6413500 |
| | | | (Q576R) | |
| rs3024677 | rs1805011 | rs3024678 | rs2234898 | rs1805016 |

LTA

| | | | | |
|---|---|---|---|---|
| rs2857708 | rs3093540 | rs4645834 | rs4947326 | rs2516312 |
| rs3093547 | rs4947327 | rs2844482 | rs3093545 | rs2844486 |
| rs2071590 | rs4248157 | rs2857601 | rs1800683 | rs9282875 |
| rs2844485 | rs2239704 | rs1799964 | rs3131637 | rs3093546 |
| rs4647198 | rs7449641 | rs909253 | rs9282876 | rs12174951 |
| rs4986978 | rs2507961 | rs3135048 | rs746868 | rs1800630 |
| rs2844484 | rs4647193 | rs2844483 | rs4647194 | rs9267499 |
| rs2857713 | rs2857706 | rs3093542 | rs2009658 | rs3093543 |
| rs3093539 | rs2071589 | rs736160 | rs1041981 | rs915654 |
| | | | (Thr26Asn) | |
| rs4647195 | rs4647191 | rs4647196 | rs4647192 | rs3093544 |

HSP70

| | | | |
|---|---|---|---|
| rs547828 | rs11557921 | rs2075800 | rs1061581 |
| rs2227956 | rs506770 | rs1008438 | rs541340 |
| (T2437C) | | | |
| rs1043618 | rs508603 | rs11557923 | rs508633 |
| rs1043620 | rs562047 | rs12190359 | rs11557924 |

TLR4

| | | | | |
|---|---|---|---|---|
| rs2149356 | rs5030714 | rs5030727 | rs4986790 | rs5030728 |
| | | | (Asp299Gly) | |
| rs2770145 | rs5030729 | rs2770144 | rs100001066 | rs5031050 |
| rs5030710 | rs11536884 | rs5030711 | rs4986791 | rs5030712 |
| | | | (Thr399Ile) | |
| rs16906079 | rs5030713 | | | |

IFNG

| | | | | |
|---|---|---|---|---|
| rs2069705 | rs2069713 | rs2069719 | rs2069724 | rs2069731 |
| rs1861494 | rs9282708 | rs2069725 | rs2069706 | rs2234685 |
| rs2069720 | rs4394909 | rs2069707 | rs1861493 | rs1042274 |
| rs2069726 | rs3814242 | rs2069714 | rs2069721 | rs2069727 |
| rs2069709 | rs2069715 | rs2069734 | rs2069710 | rs2069716 |
| rs2069722 | rs2069711 | rs2069717 | rs2234687 | rs2069712 |
| rs2069718 | rs7957366 | rs2430561 | rs3087272 | rs2069723 |
| | | (874 A/T) | | |

NFKBIL1

| | | | | |
|---|---|---|---|---|
| rs2071592 | rs2844492 | rs12181589 | rs3869143 | rs2857710 |
| (−63 T/A) | | | | |
| rs2516386 | rs2523500 | rs11756780 | rs4081553 | rs3131641 |
| rs2516385 | rs2523499 | rs2857606 | rs4947324 | rs2844490 |
| rs11962114 | rs9267491 | rs2516395 | rs4947325 | rs2844489 |
| rs2239708 | rs2516383 | rs9267493 | rs2516479 | rs2857709 |
| rs2071591 | rs7749930 | rs12526552 | rs13215091 | rs2844488 |
| rs9380262 | rs6916921 | rs12200955 | rs2516392 | rs2844487 |
| rs3219183 | rs9378162 | rs3130061 | rs2516391 | rs2857602 |
| rs9267489 | rs7754479 | rs13220163 | rs2857603 | rs2857708 |
| rs3219182 | rs11752951 | rs13220165 | rs2516390 | rs4947326 |
| rs3219181 | rs11752976 | rs2857605 | rs9501149 | rs4947327 |
| rs3219180 | rs2255798 | rs2857604 | rs9267494 | rs2844486 |
| rs2857607 | rs9267492 | rs3093949 | rs6903106 | rs2857601 |
| rs9267490 | rs3094594 | rs2239707 | rs928815 | rs2844485 |
| rs9394075 | rs2516397 | rs3219179 | rs7762619 | rs3131637 |
| rs2516384 | rs2516396 | rs2230365 | rs12663590 | rs7449641 |
| rs13190709 | rs2255899 | rs3130062 | rs7746486 | rs12174951 |
| rs2523501 | rs9394076 | rs13192469 | rs3135043 | rs3135048 |
| rs7738380 | rs12661769 | rs9469024 | rs3135042 | rs2844484 |
| rs11751074 | rs6929796 | rs4288225 | rs2523514 | |

PDGFRA

| | | | | |
|---|---|---|---|---|
| rs7682912 | rs17084062 | rs890203 | rs2412555 | rs4484359 |
| rs17084065 | rs4864861 | rs11941325 | rs12649484 | rs2114039 |
| rs4864504 | rs11942406 | rs7683707 | rs13140747 | rs4608869 |
| rs11944066 | rs12506783 | rs13114391 | rs4368668 | rs4422471 |
| rs17084050 | rs17084067 | rs4635872 | rs10029499 | rs17084051 |
| (−1630 I/D) | | | | |
| rs6850748 | rs4637467 | rs7673597 | rs1800809 | rs4864862 |
| rs7677751 | rs7673625 | rs6554162 | rs4864505 | rs7673984 |
| rs1800810 | rs4864863 | rs4352534 | rs1800813 | rs4864844 |
| rs4394060 | rs1135534 | rs7678144 | rs4508953 | rs1800812 |
| rs6554163 | rs4864857 | rs7690503 | rs6836215 | rs4864858 |
| rs7673027 | rs6554164 | rs4864859 | rs7668190 | rs11727127 |
| rs4864860 | rs7689569 | rs11937644 | rs7698425 | rs12644271 |
| rs13435164 | rs7681399 | rs7673853 | rs6554165 | rs6839108 |
| rs7679903 | rs6842432 | | | |

IL4

| | | | | |
|---|---|---|---|---|
| rs2243250 | rs2243261 | rs2243280 | rs10066662 | rs4986963 |
| (−589 C/T) | | | | |
| rs2227282 | rs2243281 | rs6885996 | rs17772853 | rs2243263 |
| rs6879348 | rs13181331 | rs2070874 | rs2243264 | rs2243282 |
| rs13161530 | rs2243251 | rs2243265 | rs2243283 | rs4426908 |
| rs4986964 | rs2243266 | rs2243284 | rs7731792 | rs2243252 |
| rs2243267 | rs2243285 | rs11749544 | rs2079102 | rs2243268 |
| rs7703922 | rs6897429 | rs734244 | rs9282745 | rs12521281 |
| rs2406539 | rs2243253 | rs9282746 | rs2243286 | rs11747814 |
| rs4996002 | rs2243270 | rs2243287 | rs10043403 | rs9327636 |
| rs2243308 | rs2243309 | rs11242122 | rs9327637 | rs2243271 |
| rs2243288 | rs11242123 | rs2243254 | rs2243272 | rs2243289 |
| rs11242124 | rs2243255 | rs2243273 | rs2243290 | rs11242125 |
| rs2243257 | rs2243274 | rs2243291 | rs6879672 | rs2243258 |
| rs2243275 | rs2243292 | rs1859139 | rs2243259 | rs2243276 |
| rs7379604 | rs1859138 | rs2227284 | rs2243277 | rs7379607 |
| rs10068370 | rs2243260 | rs2243279 | rs10066660 | rs11949166 |

MMP1

| | | | | |
|---|---|---|---|---|
| rs498186 | rs509332 | rs570662 | rs575727 | rs473509 |
| rs534191 | rs7125865 | rs7102189 | rs2075847 | rs1155764 |
| rs2000609 | rs573764 | rs470146 | rs7107224 | rs2839969 |
| rs574939 | rs1799750 | rs685265 | rs12283571 | rs542603 |
| | (−1607 1G/2G) | | | |
| rs470211 | rs12279710 | rs519806 | rs12280880 | rs533621 |
| rs2408490 | rs12285331 | rs526215 | rs470206 | rs470307 |
| rs12286876 | rs504875 | rs2105581 | rs484915 | rs634607 |
| rs12283759 | rs11225427 | rs552306 | | |

PDGFA

| | | |
|---|---|---|
| rs1800815 | no rs number His69His | rs1800814 |
| C − 26IN3T | | (C + 12IN5T) |

GCLM

| | | | | |
|---|---|---|---|---|
| rs3170633 | rs2234730 | rs12094906 | rs2273407 | rs10489605 |
| rs6703130 | rs7515991 | rs2273406 | rs12094966 | rs11165031 |
| rs12057371 | rs12401915 | rs7549683 | rs6667121 | rs12068345 |
| rs743119 | rs7513671 | rs1803636 | rs11165033 | rs736762 |
| rs10874809 | rs11165032 | rs7527855 | no rs number | rs7552401 |
| | | | (−588 C/T) | |
| rs12062047 | rs3827715 | rs6700112 | rs11589165 | rs17376966 |
| rs12040570 | rs11587480 | rs2234729 | rs12090038 | rs12062214 |
| rs6680315 | rs4598495 | rs12062216 | rs6541405 | rs12090230 |
| rs12760946 | rs12730517 | rs6692306 | rs12089163 | rs7541690 |
| rs6702045 | rs12140446 | rs718873 | rs6702064 | rs7522297 |
| rs718874 | rs2391323 | rs7517826 | rs718875 | rs2064764 |
| rs7515191 | rs12741834 | rs12143416 | rs1803635 | rs12410324 |
| rs12064127 | rs12068168 | rs2301022 | rs769211 | rs7515813 |
| rs3789453 | | | | |

OR13G1

| | |
|---|---|
| rs1151640 Ile132Val | no other genotyped snps |

IL10

| | | | | |
|---|---|---|---|---|
| rs3024498 | rs3024508 | rs1518110 | rs3024489 | rs3024510 |
| rs1878672 | rs3021094 | rs3024488 | rs3024497 | rs3024493 |
| rs3024491 | rs1800872 | rs3024496 | rs3024507 | rs3021093 |
| rs1800895 | rs5743628 | rs3024492 | rs3790622 | rs1800871 |
| rs3024495 | rs2352792 | rs3024490 | rs1800894 | rs5743627 |
| rs1554286 | rs2222202 | rs1800896 | rs3024509 | rs5743626 |
| | | (IL10-1082 A/G) | | |
| rs3021098 | rs3024494 | rs1518111 | rs3001099 | rs9282740 |
| rs3024506 | rs5743625 | | | |

AAT; SERPINA1

| | | | | |
|---|---|---|---|---|
| rs17824597 | rs1243166 | rs11846959 | rs20546 | rs17090693 |
| rs1051052 | rs17090719 | rs11558261 | rs1243168 | rs1243165 |
| rs2070709 | rs709932 | rs1884549 | rs7142803 | rs2753938 |
| rs17751614 | rs7144409 | rs1243162 | rs1243167 | rs1243164 |
| rs2749547 | rs1884548 | rs2073333 | rs2753937 | rs1885065 |
| rs1243163 | rs2854258 | rs1884547 | rs1802960 | rs17580 |
| | | | | (S allele) |
| rs1884546 | rs1303 | rs1049800 | rs9944117 | rs13170 |
| rs2230075 | rs875989 | rs12233 | rs8350 | rs877084 |
| rs12077 | rs6647 | rs877083 | rs1050520 | rs11558264 |
| rs877082 | rs1050469 | rs7141735 | rs877081 | rs1802961 |
| rs7145047 | rs11568814 | rs1802959 | rs2239652 | rs9944155 |
| rs2753939 | rs7145770 | rs11832 | rs2749521 | rs2239651 |
| rs11628917 | rs1802962 | rs11558263 | | |

ICAM1

| | | | | |
|---|---|---|---|---|
| rs1799969 | rs5030383 | rs2735439 | rs2569705 | rs5493 |
| rs281436 | rs2569697 | rs10402760 | rs5030381 | rs923366 |
| rs2075742 | rs2569706 | rs5494 | rs281437 | rs2569698 |
| rs2569707 | rs3093033 | rs3093030 | rs11669397 | rs2735441 |
| rs5495 | rs5030384 | rs901886 | rs2436545 | rs1801714 |
| rs5030385 | rs885742 | rs2436546 | rs13306429 | rs3810159 |
| rs2569699 | rs2916060 | rs2071441 | rs281438 | rs1056538 |
| rs2916059 | rs5496 | rs3093029 | rs11549918 | rs2916058 |
| rs5497 | rs2735442 | rs2569700 | rs2569708 | rs13306430 |
| rs2569693 | rs2228615 | rs12972990 | rs5498 | rs281439 |
| | | | (K469E) | |
| rs2569701 | rs735747 | rs5030400 | rs281440 | rs2569702 |
| rs885743 | rs2071440 | rs2569694 | rs2735440 | rs5499 |
| rs11575073 | rs2569703 | rs3093032 | rs2569695 | rs10418913 |
| rs1057981 | rs2075741 | rs1056536 | rs5500 | rs11575074 |
| rs2569704 | rs5501 | rs2569696 | rs11673661 | |

BAT1

| | | | | |
|---|---|---|---|---|
| rs10456058 | rs3219190 | rs3130057 | rs2523507 | rs2516485 |
| rs1048885 | rs1266079 | rs2239525 | rs2734572 | rs11264 |
| rs2844504 | rs2239526 | rs3093984 | rs9267480 | rs9267483 |
| rs16899756 | rs3130051 | rs3093978 | rs13211189 | rs2239527 |
| | | | | (−23 G/C) |
| rs2734580 | rs11543322 | rs2734583 | rs2523506 | rs3130052 |
| rs2516478 | rs2516338 | rs2523505 | rs3130053 | rs3130056 |
| rs9267484 | rs2239528 | rs2734579 | rs2734585 | rs9380261 |
| rs16899760 | rs2734578 | rs2516477 | rs9267485 | rs2523504 |
| rs2734577 | rs3853601 | rs3130058 | rs2844509 | rs3130633 |
| rs3093977 | rs2516394 | rs3130630 | rs2907768 | rs2734584 |
| rs3093975 | rs3130629 | rs2516484 | rs9267481 | rs3093974 |
| rs12662306 | rs9267478 | rs11796 | rs1129640 | rs9267487 |
| rs2734588 | rs3093948 | rs933208 | rs2251824 | rs2516483 |
| rs1055385 | rs2071596 | rs12215563 | rs2516482 | rs1055384 |
| rs2516393 | rs2071594 | rs2516481 | rs1055388 | rs2523512 |
| rs2071593 | rs3130054 | rs3131629 | rs2523511 | rs2239705 |
| rs9394074 | rs6915692 | rs3219187 | rs2523503 | rs2516480 |
| rs3131628 | rs2071595 | rs2523502 | rs2259435 | rs6915573 |
| rs2239709 | rs3093983 | rs3093976 | rs2516473 | rs2286712 |
| rs7753431 | rs9501148 | rs13206927 | rs2516475 | rs2523510 |
| rs2734587 | rs2516474 | rs2269476 | rs3093982 | rs2471826 |
| rs12665489 | rs2734586 | rs2734582 | rs11757236 | rs3130055 |
| rs929138 | rs12665501 | rs3093981 | rs2075581 | rs12178599 |
| rs3093980 | rs2734581 | rs2079170 | rs2075582 | rs2075580 |
| rs2523508 | rs3093979 | rs9267482 | rs7738430 | rs3115537 |
| rs10456396 | rs3130059 | | | |

NOS3

| | | | | |
|---|---|---|---|---|
| rs2373962 | rs2566519 | rs2853797 | rs2373961 | rs3918157 |
| rs13311166 | rs6951150 | rs3918158 | rs13310774 | rs13238512 |
| rs3918159 | rs2853798 | rs10247107 | rs2566516 | rs11974098 |
| rs10276930 | rs3918225 | rs3918166 | rs10277237 | rs3918160 |
| rs3730001 | rs12703107 | rs1800779 | rs3918167 | rs6946340 |
| rs2243311 | rs3918168 | rs6946091 | rs3918161 | rs3918169 |
| rs6946415 | rs10952298 | rs3918170 | rs10952296 | rs2070744 |
| rs3793342 | rs13309715 | rs3918226 | rs3793341 | rs10952297 |
| rs3918162 | rs1549758 | rs7784943 | rs3918163 | rs1007311 |
| rs11771443 | rs3918164 | rs9282803 | rs2243310 | rs3918165 |
| rs9282804 | rs1800783 | rs1800781 | rs1799983 | rs3918155 |
| | | | (Asp298Glu) | |
| rs13310854 | rs3918156 | rs13310763 | | |

SOD3

| | |
|---|---|
| rs1799895; Arg213Gly | within recombination hotspot |

PAI-1

| | | | | |
|---|---|---|---|---|
| rs6972498 | rs7788533 | rs2227707 | rs12536798 | rs6975620 |
| rs2227631 | rs6959121 | rs6956010 | rs1799768 | rs17135252 |
| | | | (−675/−668 4G/5G) | |
| rs12534508 | rs4729662 | rs4729664 | rs11770439 | rs2527316 |
| rs4727479 | rs2854235 | rs4729663 | rs10228765 | rs6950982 |
| rs2854225 | rs6465787 | rs2854226 | | |

MIP1A

| | | | | |
|---|---|---|---|---|
| rs8075808 | rs1719126 | rs4995343 | rs17616714 | rs5023530 |
| rs4995344 | rs7214787 | rs5023529 | rs4995345 | rs9903603 |
| rs5023528 | rs4995346 | rs1634486 | rs5023527 | rs9972917 |
| rs7213813 | rs1396785 | rs1634497 | rs8075709 | rs2522124 |
| rs7406518 | rs1634488 | rs2522125 | rs1634498 | rs1634489 |
| rs764871 | rs8076279 | rs3859285 | rs764872 | rs1634499 |
| rs3859286 | rs2376461 | rs17679109 | rs3859287 | rs2011959 |
| rs9972960 | rs1634490 | rs1719127 | rs1634500 | rs17616756 |
| rs3169944 | rs1634501 | rs5022560 | rs1049203 | rs17617023 |
| rs2136430 | rs8951 | rs1634502 | rs4319819 | rs1049200 |
| rs17617047 | rs6505505 | rs16971943 | rs8068313 | rs1620728 |
| rs1049199 | rs12602397 | rs4302099 | rs3210166 | rs1634503 |
| rs4319820 | rs1063340 | rs2687498 | rs1634491 | rs1049195 |
| rs16971970 | rs4506953 | rs1049191 | rs12452083 | rs1634492 |
| rs16971944 | rs5011009 | rs1634493 | rs8070375 | rs4636955 |
| rs4309452 | rs1049188 | rs1626203 | rs5015795 | rs16971946 |
| rs9900984 | rs1851503 | rs5029406 | rs12937093 | rs1719209 |
| rs5029407 | rs12937627 | rs16971936 | rs1049131 | rs1719135 |
| rs16971937 | rs1049121 | rs1719136 | rs1634494 | rs1049114 |
| rs1634504 | rs9893539 | rs5029408 | rs1634505 | rs12601899 |
| rs5029409 | rs11658357 | rs9916016 | rs5029410 | rs1634506 |
| rs1719123 | rs1719130 | rs16971972 | rs16971938 | rs6505506 |
| rs9913910 | rs1879917 | rs6505507 | rs1634507 | rs1719124 |
| rs1719131 | rs17679271 | rs7406217 | rs1130371 | rs8072326 |
| rs7406596 | rs16971957 | rs2072091 | rs7406597 | rs1851501 |
| rs1634508 | rs7502900 | rs1719133 | rs7220510 | rs7222527 |
| rs1634495 | rs1719134 | rs2188973 | rs16971960 | rs2188974 |
| | (+459 C/T) | | | |
| rs4995339 | rs7222850 | rs4995340 | rs7222217 | rs4995341 |
| rs1719125 | rs4995342 | | | |

MMP7

| | | | | |
|---|---|---|---|---|
| rs14983 | rs11225305 | rs12289943 | rs2847530 | rs12419959 |
| rs12788028 | rs7926218 | rs12419977 | rs10895308 | rs2187364 |
| rs11225306 | rs2408391 | rs2156528 | rs11225307 | rs11820758 |
| rs17098292 | rs10502001 | rs880197 | rs17352054 | rs12289049 |
| rs1943778 | rs11225303 | rs10750646 | rs11827824 | rs12288254 |
| rs10502002 | rs1943779 | rs7933135 | rs11225308 | rs7926470 |
| rs12575975 | rs11225304 | rs11225309 | rs17098295 | rs17098317 |
| rs6590970 | rs10895306 | rs1996352 | rs10895307 | rs12418158 |
| rs2408390 | rs2701982 | rs17881472 | rs12419636 | rs17880821 |
| | | | | (−181 A/G) |
| rs17098306 | rs4543946 | rs12285347 | rs17098318 | |

Cathepsin G

| | | | | | |
|---|---|---|---|---|---|
| rs9671740 | rs34792401 | rs12588201 | rs10148261 | rs6573865 | rs8007970 |
| rs11848310 | rs35614744 | rs35345293 | rs4537944 | rs2332397 | rs8012655 |
| rs12889110 | rs10311660 | rs4981537 | rs36079901 | rs2332398 | rs1956907 |
| rs12587099 | rs12147842 | rs4981538 | rs9989246 | rs3891906 | rs1956906 |
| rs12879629 | rs9671474 | rs4981539 | rs9989184 | rs3891905 | rs35316440 |
| rs35776728 | rs1756602 | rs4981540 | rs34712519 | rs11851298 | rs28778409 |
| rs35270658 | rs11157885 | rs34432073 | rs12100969 | rs3861508 | rs8008954 |
| rs34377408 | rs10143176 | rs35884608 | rs10144984 | rs7142675 | rs2093256 |
| rs34310125 | rs12436232 | rs4981541 | rs10145731 | rs11301449 | rs11158781 |
| rs35426278 | rs5007574 | rs9646161 | rs28806511 | rs28441642 | rs7157694 |
| rs2216900 | rs28878775 | rs4982959 | rs10145286 | rs2011607 | rs8005739 |
| rs12435455 | rs35112976 | rs11158761 | rs28803584 | rs11158778 | rs7143951 |
| rs10138356 | rs34065379 | rs4632067 | rs9652365 | rs11845858 | rs7144090 |
| rs4644778 | rs12433770 | rs34318040 | rs12888328 | rs2224426 | rs7144096 |
| rs11850630 | rs11159232 | rs8010555 | rs12887710 | rs4982968 | rs7144104 |
| rs34841597 | rs12882370 | rs10148816 | rs8006887 | rs35704637 | rs34651242 |
| rs12879894 | rs36126615 | rs10133351 | rs8007599 | rs1951132 | rs10136912 |
| rs4375584 | rs9672023 | rs12890682 | rs9652294 | rs1951131 | rs9323523 |
| rs800849 | rs9944068 | rs4553550 | rs7155992 | rs1951130 | rs28722668 |
| rs34042254 | rs9944069 | rs4537943 | rs12888151 | rs1951129 | rs35959655 |
| rs34545435 | rs1956918 | rs12433996 | rs34436261 | rs3079309 | rs34997518 |
| rs12885502 | rs1956917 | rs34092671 | rs36100678 | rs34634241 | rs17257069 |
| rs12885756 | rs1956916 | rs8005125 | rs35239717 | rs11849152 | rs6573871 |
| rs12885748 | rs761988 | rs35584604 | rs12587746 | rs1956911 | rs17105013 |
| rs12885747 | rs927281 | rs1956915 | rs34035545 | rs12888496 | rs28479407 |
| rs12885233 | rs12434912 | rs10873216 | rs4420452 | rs1956910 | rs12100907 |
| rs34449575 | rs28656464 | rs34656084 | rs721134 | rs1956909 | rs11158791 |
| rs34780137 | rs35275136 | rs34416904 | rs10130107 | rs12588702 | rs1956912 |
| rs12886566 | rs4982956 | rs28496049 | rs3901388 | rs2332399 | rs1956908 |
| rs11850400 | rs7148705 | rs12589666 | rs35220969 | rs1951128 | rs1304909 |
| rs12882368 | rs2007323 | rs7151899 | rs3861506 | rs2332400 | rs8021299 |
| rs12882089 | rs2007316 | rs7149913 | rs3861507 | rs28768164 | rs7155452 |
| rs12896244 | rs927278 | rs7149925 | rs3905107 | rs10144882 | rs2332401 |
| rs35106724 | rs8007723 | rs7156977 | rs1956914 | rs1951127 | rs8007285 |
| rs35193389 | rs6573850 | rs4982960 | rs1956913 | rs8022028 | rs11849224 |
| rs12432795 | rs34858090 | rs7141080 | rs987630 | rs10131084 | rs6573874 |
| rs12432796 | rs3079301 | rs33993912 | rs987629 | rs10139948 | rs12587351 |
| rs34281648 | rs35839512 | rs4982961 | rs987628 | rs10139332 | rs12587425 |
| rs800850 | rs34614283 | rs12878586 | rs11158770 | rs1951125 | rs4982969 |
| rs34013113 | rs4982957 | rs8004636 | rs932617 | rs33995132 | rs12587539 |
| rs11851718 | rs2208447 | rs10130100 | rs932616 | rs1951124 | rs10601342 |
| rs12433789 | rs7146705 | rs11849539 | rs1885106 | rs8007117 | rs35533698 |
| rs13379195 | rs9806075 | rs35778376 | rs8008866 | rs8006658 | rs10601343 |
| rs13379433 | rs34657109 | rs8008632 | rs4982964 | rs34042582 | rs4592564 |
| rs4287482 | rs7151943 | rs11520369 | rs4982965 | rs34002761 | rs8019787 |
| rs34264349 | rs7150627 | rs34079088 | rs4982966 | rs8007445 | rs1885597 |
| rs4417480 | rs4981536 | rs4982962 | rs10134629 | rs34071722 | Asn 125Ser |
| rs2792215 | rs4982958 | rs4981542 | rs8003038 | rs12890487 | rs17105289 |
| rs10313155 | rs11351813 | rs4982963 | rs8017569 | rs12890488 | rs34281853 |
| rs34640640 | rs10712328 | rs36074365 | rs10137473 | rs12890064 | rs2070697 |

CX3CR1

| | | | | | |
|---|---|---|---|---|---|
| rs4234139 | rs11707528 | rs17038647 | rs2669850 | rs4676621 | rs34570795 |
| rs11129819 | rs17793056 | rs4676622 | rs34841495 | rs11129820 | rs2669851 |
| rs12491311 | rs6599018 | rs9826296 | rs2853714 | rs34980214 | rs11709600 |
| rs17038663 | rs36040453 | rs35840437 | rs34991361 | rs7636125 | rs34864276 |
| rs35425914 | rs9847920 | rs11710546 | rs13088991 | rs13078589 | rs17038640 |
| rs17038674 | rs13099085 | rs9869871 | rs1050592 | rs34681148 | rs10695501 |
| rs3732378 | rs35007101 | rs35540291 | rs3732379 | rs6790767 | rs4016736 |
| | | | (I249V) | | |
| rs4986872 | rs9882352 | rs11711922 | rs17038679 | rs938206 | rs4676623 |
| rs3732380 | rs938207 | rs34481570 | rs7645269 | rs11706384 | rs4676487 |
| rs4271863 | rs11711391 | rs35291973 | rs35502141 | rs7649301 | rs1877563 |
| rs34743375 | rs17038638 | rs11713282 | rs12634272 | rs13315491 | rs17038645 |
| rs34139145 | | | | | |

NOD2

| | | | | | |
|---|---|---|---|---|---|
| rs6596 | rs34430742 | rs1078327 | rs8061960 | rs12929234 | rs17314544 |
| rs35435054 | rs4785224 | rs5743274 | rs34192073 | rs34552113 | rs17223195 |
| rs35581802 | rs5743261 | rs35422070 | rs2066847 | rs35899583 | rs13337656 |
| rs8054275 | rs5743262 | rs1861759 | rs5743293 | rs12324931 | rs13338860 |
| rs13339019 | rs5743263 | rs5743275 | rs5743294 | rs3135501 | rs12599914 |
| rs35973532 | rs8046608 | rs5743276 | rs34829738 | rs3135502 | rs35234675 |
| rs1131716 | rs35378728 | rs2066844 | rs2357791 | rs3135503 | rs16948819 |
| rs17846633 | rs5743264 | rs5743277 | rs7359452 | rs34683241 | rs7200370 |
| rs34428900 | rs5743265 | rs35285618 | rs7203344 | rs11861521 | rs4785226 |
| rs2302723 | rs5743266 | rs5743278 | rs5743295 | rs4785450 | rs11646600 |
| rs7195707 | rs2076752 | rs6413461 | rs5743296 | rs11862710 | rs17223257 |
| rs9888893 | rs5743267 | rs3813758 | rs35980453 | rs11862720 | rs11865799 |
| rs7191692 | rs8061316 | rs5743279 | rs3135499 | rs1990752 | rs35072258 |
| rs10600956 | rs8061636 | rs5743280 | rs5743297 | rs11864090 | rs11866167 |
| rs35509283 | rs34456998 | rs5743281 | rs5743298 | rs34464167 | rs10451132 |
| rs12448380 | rs16948754 | rs5743282 | rs5743299 | rs34620046 | rs3064638 |
| rs12445637 | rs34627107 | rs4785225 | rs3135500 | rs8062105 | rs2066852 |
| rs7202124 | rs35085911 | rs16948773 | rs5743300 | rs34321598 | rs2302759 |
| rs2270369 | rs34281847 | rs9931711 | rs8056611 | rs16948792 | rs7194167 |
| rs2270368 | rs7206340 | rs17313265 | rs35111385 | rs34741614 | rs34233862 |
| rs35929558 | rs36034720 | rs34912053 | rs2357792 | rs11859047 | rs12444259 |
| rs34828195 | rs35701609 | rs35263569 | rs12600253 | rs11863594 | rs28705891 |
| rs2287195 | rs2076753 | rs10680678 | rs12598306 | rs11864698 | rs28654666 |
| rs2066848 | rs34936594 | rs35322998 | rs7205423 | rs1477176 | rs11382774 |
| rs36094725 | rs35095295 | rs11646168 | rs718226 | rs35967774 | rs34593836 |
| rs11336436 | rs34684955 | rs35431588 | rs34963149 | rs2066851 | rs2160683 |
| rs10709169 | rs2067085 | rs9925315 | rs11646242 | rs1592639 | rs3743781 |
| rs4785223 | rs16948755 | rs5743284 | rs35151581 | rs9925070 | rs9646285 |
| rs9926569 | rs34939799 | rs5743285 | rs7498888 | rs11863544 | rs16948829 |
| rs9939349 | rs2111235 | rs751271 | rs5816718 | rs3064635 | rs34088926 |
| rs28651300 | rs35617724 | rs748855 | rs11323644 | rs12933741 | rs16948836 |
| rs8062727 | rs2111234 | rs1861758 | rs8060765 | rs12933742 | rs17314948 |
| rs8044354 | rs7190413 | rs13332952 | rs16948789 | rs11863916 | rs34262697 |
| rs8043770 | rs7206582 | rs7198979 | rs17313747 | rs5816721 | rs7184210 |
| rs10459815 | rs8045009 | rs1861757 | rs5816719 | rs11859674 | rs34917190 |
| rs9302752 | rs6500328 | rs7203691 | rs35360138 | rs1420873 | rs34015619 |
| rs13331872 | rs7500036 | rs5743286 | rs5816720 | rs8053457 | rs34678551 |
| rs1420685 | rs8057341 | rs5743287 | rs34562455 | rs6500329 | rs35812259 |
| rs5816713 | rs35863026 | rs11319364 | rs3064634 | rs7500289 | rs6500333 |
| rs33925268 | rs12918060 | rs34103974 | rs751919 | rs12924216 | rs17223592 |
| rs4027240 | rs7204911 | rs10521209 | rs3743782 | rs1548989 | rs11866769 |
| rs35554928 | rs7500826 | rs2066845 | rs11353661 | rs16948808 | rs1861762 |
| | | Gly 881 Arg G/C) | | | |
| rs2004804 | rs4785449 | rs5743288 | rs7199842 | rs4283227 | rs7187386 |
| rs12448915 | rs12922299 | rs5743289 | rs1362698 | rs1420872 | rs8051573 |
| rs5816714 | rs11649521 | rs8063130 | rs2216313 | rs4785451 | rs35224296 |
| rs5816715 | rs13339578 | rs2076756 | rs11364818 | rs6500331 | rs3922267 |
| rs34609432 | rs17221417 | rs12920425 | rs10655305 | rs11644525 | rs3087557 |
| rs11297087 | rs13331327 | rs12920040 | rs35620436 | rs35189427 | rs1054987 |
| rs34919724 | rs11642482 | rs12920558 | rs11451496 | rs16948810 | rs9635531 |
| rs34746653 | rs11642646 | rs11326665 | rs1548990 | rs16948811 | rs11374168 |
| rs34550909 | rs17312836 | rs11292073 | rs7195766 | rs16948813 | rs33995398 |
| rs5816716 | rs5743268 | rs11423748 | rs9940175 | rs6500332 | rs4785452 |
| rs34235838 | rs5743269 | rs12919099 | rs8060598 | rs17222902 | rs4785453 |
| rs3064632 | rs5743270 | rs12920721 | rs7198188 | rs1420871 | rs4785227 |
| rs1981760 | rs12925051 | rs2076755 | rs7187352 | rs2302760 | rs4785454 |
| rs8063362 | rs12929565 | rs5743290 | rs34564491 | rs7192397 | rs4785455 |
| rs9933594 | rs13380733 | rs5743291 | rs12599808 | rs17314341 | rs3064640 |
| rs1362632 | rs13380741 | rs11642651 | rs11271535 | rs12597446 | rs13332720 |
| rs12926429 | rs11647841 | rs1861756 | rs7200535 | rs3785141 | rs16948850 |
| rs35831008 | rs34409335 | rs749910 | rs13336419 | rs13333006 | rs6500334 |
| rs4785448 | rs34133110 | rs34333043 | rs3785142 | rs3785140 | rs5816722 |
| rs11647143 | rs10451131 | rs2066846 | rs7342808 | rs9938976 | rs3064642 |
| rs34981889 | rs2066842 | rs5816717 | rs7342715 | rs8061821 | rs6500335 |
| rs7194886 | rs35090774 | rs4990643 | rs28454013 | rs8062540 | rs16948851 |
| rs34231814 | rs5743271 | rs1077861 | rs7197362 | rs16948817 | rs3135504 |
| rs11645448 | rs7498256 | rs34810706 | rs12934597 | rs8047910 | rs7189846 |
| rs35832802 | rs5743272 | rs5743292 | rs12930153 | rs4027241 | rs7205760 |
| rs5743259 | rs5743273 | rs9921146 | rs12934724 | rs34145774 | rs8049077 |
| rs5743260 | rs2076754 | rs11645386 | rs12934730 | rs35896215 | rs1861761 |
| rs2066850 | rs2066843 | rs7187857 | rs12929222 | rs2111435 | rs12925755 |

TIMP1

| | | | | | |
|---|---|---|---|---|---|
| rs2858769 | rs13440654 | rs2097423 | rs35754459 | rs34026683 | rs5952438 |
| rs35510014 | rs17147652 | rs5906436 | rs2854412 | rs1984392 | rs7064051 |
| rs34644595 | rs3211164 | rs4824621 | rs34987183 | rs35777532 | rs12010140 |
| rs10701204 | rs35895268 | rs4824622 | rs34465989 | rs28764016 | rs12559303 |
| rs35338820 | rs28764017 | rs34324592 | rs1050151 | rs1043428 | rs5953061 |
| rs34865437 | rs5953060 | rs5953062 | rs35946819 | rs4898 | rs13441207 |
| | | | | (372 T/C) | |
| rs34663452 | rs35209437 | rs5953063 | rs1050175 | rs6609533 | rs5906437 |
| rs10066 | rs34220495 | rs3921110 | rs35136843 | rs1803571 | rs34949562 |
| rs2855135 | rs11551797 | rs6520281 | rs2854414 | rs17850165 | rs34910886 |
| rs2855136 | rs1062849 | rs5953065 | rs2854415 | rs2070584 | rs9887335 |
| rs2854416 | rs6609534 | rs34207832 | rs2854417 | rs34478552 | rs35891328 |
| rs723556 | rs5906434 | rs12007301 | rs12394306 | rs6520278 | rs6609539 |
| rs35093912 | rs6520279 | rs5953066 | rs5953059 | rs7886171 | rs6608733 |
| rs34576985 | rs12556415 | rs4824424 | rs6609532 | rs5905614 | rs4824623 |
| rs4282692 | rs5905615 | rs6608734 | rs13440825 | rs5906435 | |

### INDUSTRIAL APPLICATION

The present invention is directed to methods for assessing a subject's risk of developing ACS. The methods comprise the analysis of polymorphisms herein shown to be associated with increased or decreased risk of developing ACS, or the analysis of results obtained from such an analysis. The use of polymorphisms herein shown to be associated with increased or decreased risk of developing ACS in the assessment of a subject's risk are also provided, as are nucleotide probes and primers, kits, and microarrays suitable for such assessment. Methods of treating subjects having the polymorphisms herein described are also provided. Methods for screening for compounds able to modulate the expression of genes associated with the polymorphisms herein described are also provided.

All patents, publications, scientific articles, and other documents and materials referenced or mentioned herein are indicative of the levels of skill of those skilled in the art to which the invention pertains, and each such referenced document and material is hereby incorporated by reference to the same extent as if it had been incorporated by reference in its entirety individually or set forth herein in its entirety. Applicants reserve the right to physically incorporate into this specification any and all materials and information from any such patents, publications, scientific articles, web sites, electronically available information, and other referenced materials or documents.

The specific methods described herein are representative of various embodiments or preferred embodiments and are exemplary only and not intended as limitations on the scope of the invention. Other objects, aspects, examples and embodiments will occur to those skilled in the art upon consideration of this specification, and are encompassed within the spirit of the invention as defined by the scope of the claims. It will be readily apparent to one skilled in the art that varying substitutions and modifications can be made to the invention disclosed herein without departing from the scope and spirit of the invention. The invention illustratively described herein suitably can be practiced in the absence of any element or elements, or limitation or limitations, which is not specifically disclosed herein as essential. Thus, for example, in each instance herein, in embodiments or examples of the present invention, any of the terms “comprising”, “consisting essentially of”, and “consisting of” may be replaced with either of the other two terms in the specification, thus indicating additional examples, having different scope, of various alternative embodiments of the invention. Also, the terms “comprising”, “including”, containing”, etc. are to be read expansively and without limitation. The methods and processes illustratively described herein suitably may be practiced in differing orders of steps, and that they are not necessarily restricted to the orders of steps indicated herein or in the claims. It is also that as used herein and in the appended claims, the singular forms “a,” “an,” and “the” include plural reference unless the context clearly dictates otherwise. Thus, for example, a reference to “a host cell” includes a plurality (for example, a culture or population) of such host cells, and so forth. Under no circumstances may the patent be interpreted to be limited to the specific examples or embodiments or methods specifically disclosed herein. Under no circumstances may the patent be interpreted to be limited by any statement made by any Examiner or any other official or employee of the Patent and Trademark Office unless such statement is specifically and without qualification or reservation expressly adopted in a responsive writing by Applicants.

The terms and expressions that have been employed are used as terms of description and not of limitation, and there is no intent in the use of such terms and expressions to exclude any equivalent of the features shown and described or portions thereof, but it is recognized that various modifications are possible within the scope of the invention as claimed. Thus, it will be understood that although the present invention has been specifically disclosed by preferred embodiments and optional features, modification and variation of the concepts herein disclosed may be resorted to by those skilled in the art, and that such modifications and variations are considered to be within the scope of this invention.

1. A method of determining a subject's risk of developing acute coronary syndrome (ACS), comprising analysing a sample from said subject for the presence or absence of at least one polymorphism selected from the group consisting of:
−1903 A/G in the gene encoding Chymase 1 (CMA1);
−82 A/G in the gene encoding Matrix metalloproteinase 12 (MMP12);
Ser52Ser (223 C/T) in the gene encoding Fibroblast growth factor 2 (FGF2);
Q576R AJG in the gene encoding Interleukin 4 receptor alpha (IL4RA);
HOM T2437C in the gene encoding Heat Shock Protein 70 (HSP 70);
874 AJT in the gene encoding Interferon γ (IFNG);
−589 C/T in the gene encoding Interleukin 4 (IL-4);
−1084 A/G (−1082) in the gene encoding Interleukin 10 (IL-10);
Arg213Gly C/G in the gene encoding Superoxide dismutase 3 (SOD3);
459 C/T Intron I in the gene encoding Macrophage inflammatory protein 1 alpha (MIP1A);
Asn 125 Ser A/G in the gene encoding Cathepsin G;
I249V C/T in the gene encoding Chemokine (CX3C motif) receptor 1 (CX3CR1);
Gly 881 Arg G/C in the gene encoding Caspase (NOD2); or
372 T/C in the gene encoding Tissue inhibitor of metalloproteinase 1 (TIMP1);
−509 C/T in the gene encoding Transforming growth factor β1 (TGFB1);
Thr26Asn AJC in the gene encoding Lymphotoxin α (LTA);
Asp299Gly A/G in the gene encoding Toll-like Receptor 4 (TLR4);
Thr399Ile C/T in the gene encoding TLR4;
−63 T/A in the gene encoding Nuclear factor of kappa light polypeptide gene enhancer in B-cells inhibitor-like 1 (NFKBIL1);
1630 Ins/Del (AACTT/Del) in the gene encoding Platelet derived growth factor receptor alpha (PDGFRA);
−1607 1G/2G (Del/G) in the gene encoding Matrix metalloproteinase 1 (MMP1);
12 IN 5 C/T in the gene encoding Platelet derived growth factor alpha (PDGFA);
−588 C/T in the gene encoding Glutamate-cysteine ligase modifier subunit (GCLM);
Ile132Val A/G in the gene encoding Olfactory receptor analogue OR13G1 (OR13G1);
Glu288Val A/T (M7S) in the gene encoding alpha 1-antitrypsin (α1-AT);
K469E A/G in the gene encoding Intracellular adhesion molecule 1 (ICAM1);
−23 C/G in the gene encoding HLA-B associated transcript 1 (BAT1);
Glu298Asp G/T in the gene encoding Nitric Oxide synthase 3 (NOS3);
−668 4G/5G in the gene encoding Plasminogen activator inhibitor 1 (PAI-I);
−181 A/G in the gene encoding Matrix metalloproteinase 7 (MMP7); and
one or more polymorphisms which are in linkage disequilibrium with any one of said at least one polymorphism;
wherein the presence or absence of said at least one polymorphism is indicative of the subject's risk of developing ACS.

2-4. (canceled)

5. The method according to claim 1, wherein the presence of at least one polymorphism is indicative of a reduced risk of developing ACS, and wherein said at least one polymorphism is selected from the group consisting of:
the Ser52Ser (223 C/T) CC genotype in the gene encoding FGF2;
the Q576R A/G AA genotype in the gene encoding IL4RA;
the Thr26Asn A/C CC genotype in the gene encoding LTA;
the Horn T2437C CC or CT genotype in the gene encoding HSP70;
the Asp299Gly A/G AG or GG genotype in the gene encoding TLR4;
the Thr399Ile C/T CT or TT genotype in the gene encoding TLR4;
the 874 A/T TT genotype in the gene encoding IFNG;
the −63 T/A AA genotype in the gene encoding NFKBIL1;
the −1630 Ins/Del (AACTT/Del) Ins/Del or Del/Del genotype in the gene encoding PDGFRA;
the −589 C/T CT or TT genotype in the gene encoding IL-4;
the −588 C/T CC genotype in the gene encoding GCLM;
the −1084 A/G GG genotype in the gene encoding IL-10;
the K469E A/G AA genotype in the gene encoding ICAM1;
the −23 C/G GG genotype in the gene encoding BAT1;
the Glu298Asp G/T GG genotype in the gene encoding NOS3;
the Arg213Gly C/G CG or GG genotype in the gene encoding SOD3;
the −668 4G/5G 5G5G genotype in the gene encoding PAI-I;
the −181 A/G GG genotype in the gene encoding MMP7;
the Asn 125 Ser AG or GG genotype in the gene encoding Cathepsin G; and
372 T/C TT genotype in the gene encoding TIMP1.

6. The method according to claim 1, wherein the presence of at least one polymorphism is indicative of an increased risk of developing ACS and wherein said at least one polymorphism is selected from the group consisting of:
the −1903 A/G GG genotype in the gene encoding CMA1;
the −509 C/T CC genotype in the gene encoding TGFB1;
the −82 A/G GG genotype in the gene encoding MMP12;
the Ser52Ser (223 C/T) CT or TT genotype in the gene encoding FGF2;
the Q576R A/G GG genotype in the gene encoding IL4RA;
the Horn T2437C TT genotype in the gene encoding HSP70;
the Asp299Gly A/G AA genotype in the gene encoding TLR4;
the Thr399Ile C/T CC genotype in the gene encoding TLR4;
the −1630 Ins/Del (AACTT/Del) Ins Ins (AACTT AACTT) genotype in the gene encoding PDGFRA;
the −589 C/T CC genotype in the gene encoding IL4;
the −1607 1G/2G (Del/G) Del Del (IG IG) genotype in the gene encoding MMP1;
the 12 IN5 C/T TT genotype in the gene encoding PDGFA;
the −588 C/T CT or TT genotype in the gene encoding GCLM;
the Ile132Val A/G AA genotype in the gene encoding OR13G1;
the Glu288Val A/T (M/S) AT or TT (MS or SS) genotype in the gene encoding α1-AT;
the +459 C/T Intron 1 CT or TT genotype in the gene encoding MIP1A;
the Asn 125 Ser AA genotype in the gene encoding Cathepsin G;
the I249V TT genotype in the gene encoding CX3CR1;
the GIy 881 Arg G/C CC or CG genotype in the gene encoding NOD2; and
the 372 T/C CC genotype in the gene encoding TIMP1.

7. A method of assessing a subject's risk of developing ACS, said method comprising the steps:
(i) determining the presence or absence of at least one protective polymorphism associated with a reduced risk of developing ACS and
(ii) in the absence of at least one protective polymorphisms, determining the presence or absence of at least one susceptibility polymorphism associated with an increased risk of developing ACS; wherein the presence of one or more of said protective polymorphisms is indicative of a reduced risk of developing ACS, and the absence of at least one protective polymorphism in combination with the presence of at least one susceptibility polymorphism is indicative of an increased risk of developing ACS.

8. The method according to claim 7 wherein said at least one protective polymorphism is selected from the group consisting of:
the Ser52Ser (223 C/T) CC genotype in the gene encoding FGF2;
the Q576R A/G AA genotype in the gene encoding IL4RA;
the Thr26Asn AJC CC genotype in the gene encoding LTA;
the Horn T2437C CC or CT genotype in the gene encoding HSP70;
the Asp299Gly A/G AG or GG genotype in the gene encoding TLR4;
the Thr399Ile C/T CT or TT genotype in the gene encoding TLR4;
the 874 A/T TT genotype in the gene encoding IFNG;
the −63 T/A AA genotype in the gene encoding NFKBIL1;
the −1630 Ins/Del (AACTT/Del) Ins/Del or Del/Del genotype in the gene encoding PDGFRA;
the −589 C/T CT or TT genotype in the gene encoding IL-4;
the −588 C/T CC genotype in the gene encoding GCLM;
the −1084 A/G GG genotype in the gene encoding IL-10;
the K469E A/G AA genotype in the gene encoding ICAM1;
the −23 C/G GG genotype in the gene encoding BAT1;
the Glu298Asp G/T GG genotype in the gene encoding NOS3;
the Arg213Gly C/G CG or GG genotype in the gene encoding SOD3;
the −668 4G/5G 5G5G genotype in the gene encoding PAI-I;
the −181 A/G GG genotype in the gene encoding MMP7;
the Asn 125 Ser AG or GG genotype in the gene encoding Cathepsin G; and
372 T/C TT genotype in the gene encoding TIMP.

9. The method according to claim 7, wherein said at least one susceptibility polymorphism is a genotype selected from the group consisting of:
the −1903 A/G GG genotype in the gene encoding CMA1;
the −509 C/T CC genotype in the gene encoding TGFB1;
the −82 A/G GG genotype in the gene encoding MMP12;
the Ser52Ser (223 C/T) CT or TT genotype in the gene encoding FGF2;
the Q576R A/G GG genotype in the gene encoding IL4RA;
the Horn T2437C TT genotype in the gene encoding HSP70;
the Asp299Gly A/G AA genotype in the gene encoding TLR4;
the Thr399Ile C/T CC genotype in the gene encoding TLR4;
the −1630 Ins/Del (AACTT/Del) Ins Ins (AACTT AACTT) genotype in the gene encoding PDGFRA;
the −589 C/T CC genotype in the gene encoding IL4;
the −1607 1G/2G (Del/G) Del Del (IG IG) genotype in the gene encoding MMP1;
the 12 IN5 C/T TT genotype in the gene encoding PDGFA;
the −588 C/T CT or TT genotype in the gene encoding GCLM;
the Ile132Val A/G AA genotype in the gene encoding OR13G1;
the Glu288Val A/T (M/S) AT or TT (MS or SS) genotype in the gene encoding α1-AT;
the +459 C/T Intron 1 CT or TT genotype in the gene encoding MIP1A;
the Asn 125 Ser AA genotype in the gene encoding Cathepsin G;
the I249V TT genotype in the gene encoding CX3CR1;
the GIy 881 Arg G/C CC or CG genotype in the gene encoding NOD2; and
the 372 T/C CC genotype in the gene encoding TIMP.

10. The method according to claim 7, wherein the presence of two or more protective polymorphisms irrespective of the presence of one or more susceptibility polymorphisms is indicative of reduced risk of developing ACS.

11. The method according to claim 7, wherein in the absence of a protective polymorphism the presence of one or more susceptibility polymorphisms is indicative of an increased risk of developing ACS.

12. The method according to claim 7, wherein the presence of two or more susceptibility polymorphisms is indicative of an increased risk of developing ACS.

13. (canceled)

14. The method according to claim 1, wherein said method further comprises:
performing an analysis of at least one epidemiological risk factors.

15. A method of determining a subject's risk of developing acute coronary syndrome (ACS), said method comprising the steps:
(i) obtaining the result of one or more genetic tests of a sample from said subject; and
(ii) analysing the result for the presence or absence of at least one polymorphism selected from the group consisting of:
−1903 AJG in the gene encoding Chymase 1 (CMA1);
−82 AJG in the gene encoding Matrix metalloproteinase 12 (MMP12);
Ser52Ser (223 C/T) in the gene encoding Fibroblast growth factor 2 (FGF2);
Q576R AJG in the gene encoding Interleukin 4 receptor alpha (IL4RA);
HOM T2437C in the gene encoding Heat Shock Protein 70 (HSP 70);
874 AJT in the gene encoding Interferon γ (IFNG);
−589 C/T in the gene encoding Interleukin 4 (IL-4);
−1084 AJG (−1082) in the gene encoding Interleukin 10 (IL-10);
Arg213Gly C/G in the gene encoding Superoxide dismutase 3 (SOD3);
459 C/T Intron I in the gene encoding Macrophage inflammatory protein 1 alpha (MIP1A);
Asn 125 Ser A/G in the gene encoding Cathepsin G;
I249V C/T in the gene encoding Chemokine (CX3C motif) receptor 1 (CX3CR1);
GIy 881 Arg G/C in the gene encoding Caspase (NOD2);
372 T/C in the gene encoding Tissue inhibitor of metalloproteinase 1 (TIMP1); and
one or more polymorphisms which are in linkage disequilibrium with any one said at least one polymorphism;
wherein a result indicating the presence or absence of said at least one polymorphism is indicative of the subject's risk of developing ACS.

16. The method according to claim 15, wherein a result indicating the presence of at least one polymorphism is indicative of a reduced risk of developing ACS, and wherein said at least one polymorphism is selected from the group consisting of:
the Ser52Ser (223 C/T) CC genotype in the gene encoding FGF2;
the Q576R A/G AA genotype in the gene encoding IL4RA;
the Horn T2437C CC or CT genotype in the gene encoding HSP70;
the 874 A/T TT genotype in the gene encoding IFNG;
the −589 C/T CT or TT genotype in the gene encoding IL-4;
the −1084 A/G GG genotype in the gene encoding IL-10;
the Arg213Gly C/G CG or GG genotype in the gene encoding SOD3;
the Asn 125 Ser AG or GG genotype in the gene encoding Cathepsin G; and
372 T/C TT genotype in the gene encoding TIMP1.

17. The method according to claim 15 wherein a result indicating the presence of at least one polymorphism is indicative of a reduced risk of developing ACS, and wherein said at least one polymorphism is selected from the group consisting of:
the −1903 A/G GG genotype in the gene encoding CMA1;
the −82 A/G GG genotype in the gene encoding MMP12;
the +459 C/T Intron 1 CT or TT genotype in the gene encoding MIP1A;
the Asn 125 Ser AA genotype in the gene encoding Cathepsin G;
the I249V TT genotype in the gene encoding CX3CR1;
the GIy 881 Arg G/C CC or CG genotype in the gene encoding NOD2; and
the 372 T/C CC genotype in the gene encoding TIMP1.

18-26. (canceled)

27. A method for screening for compounds that modulate the expression and/or activity of a gene, wherein the expression and/or activity of said gene is upregulated or down-regulated when associated with a susceptibility or protective polymorphism selected from the group defined in claim 2 or claim 3, said method comprising the steps of:
contacting a candidate compound with a cell comprising a susceptibility or protective polymorphism which has been determined to be associated with the upregulation or downregulation of expression and/or activity of a gene; and
measuring a level of expression and/or activity of said gene following contact with said candidate compound,
wherein a change in the level of expression and/or activity of said gene after the contacting step as compared to before the contacting step is indicative of the ability of the candidate compound to modulate the expression and/or activity of said gene.

28. The method according to claim 27, wherein said cell is a human vascular cell which has been pre-screened to confirm the presence of said polymorphism.

29. (canceled)

30. The method according to claim 27, wherein said cell comprises a susceptibility polymorphism associated with upregulation of expression and/or activity of said gene and said screening is for candidate compounds which downregulate expression and/or activity of said gene.

31. The method according to claim 27, wherein said cell comprises a susceptibility polymorphism associated with downregulation of expression and/or activity of said gene and said screening is for candidate compounds which upregulate expression and/or activity of said gene.

32. The method according to claim 27, wherein said cell comprises a protective polymorphism associated with upregulation of expression and/or activity of said gene and said screening is for candidate compounds which further upregulate expression and/or activity of said gene.

33. The method according to claim 27, wherein said cell comprises a protective polymorphism associated with downregulation of expression and/or activity of said gene and said screening is for candidate compounds which further downregulate expression and/or activity of said gene.

34-40. (canceled)

41. A method of assessing the likely responsiveness of a subject predisposed to or diagnosed with acute coronary syndrome (ACS) to a prophylactic or therapeutic treatment, which treatment involves restoring the physiologically active concentration of a product of gene expression to be within a range which is normal for the age and sex of the subject, which method comprises detecting in said subject the presence or absence of a susceptibility polymorphism selected from the group defined in claim 3 which when present either upregulates or downregulates expression of said gene such that the physiological active concentration of the expressed gene product is outside said normal range, wherein the detection of the presence of said polymorphism is indicative of the subject likely responding to said treatment.

42-50. (canceled)
